SEQUENCE LISTING (14 sequences)

SEQ ID NO: 1 - DNA, 2009 bp, Artificial Sequence
  Description of Artificial Sequence Synthetic Clone ID 2B-17
tcaacgctctcaaaatcccctaaagataagattgacccgcgggacctcctaatccccttaattctcttcctgtctctcaaaggggccagatccgcagcacccggctccagccctcaccaggtctacaacattacctgggaagtgaccaatggggatcaggaggcagtatgggcaatatcaggcaaccaccctctgtggacttggtggccagacctcaccccagatttgtgtatgttagctctcagtgggccgccccactgggggctagaatatcaggccccctattcctcgcccccggggcccccttgttgctcagggagcagcgggaacagtgcaggctgtttcagagactgcgacgagcccttgacctccctcacccctcggtgcaacactgcctggaacagacttaagctagaccaggtaactcataaatcaagtgagggattttatgtctgccccgggccacatcgcccccgggaagccaagtcctgtggaggtccagattccttctactgtgcctcttggggctgcgagacaaccggtagagtatactggaagccctcctcctcttgggactacatcacagtagacaacaatctcaccactaaccaggctgttaagacaataagtggtgcaatcccttggctatccggtttacaaacaccgggagacaggtcacctcatggacaactggacactattggggtctacgtctttatgtcactgggaaggacccggggcttactttcgggatcagactcaaatatcaaaatctaggacctcgggtcccaataggaccaaaccccgtcctggcagaccaactttcgttcccgctacctaatccccaacccaaacctgccaagtctccccccgcctctaattcgactcccacattgatttccccgtcccccactcccactcagcccccgccagcaggaacgggagacaggttactaaatctagtacagggagcttaccaggcactcaacttaaccaaccctgataaaactcaagagtgctggttatgcctagtgtctggacccccctattacgagggagttgcggtcctaggtacttattccaaccatacctctgccccagctaactgctccacggcctcccaacacaagttgaccctgtccgaagtgactggacggggactctgcataggaacagtcccaaaaactcaccaggccctgtgcaacactacccttaagacaggcaaagggtcttactatctagttgcccccgcaggaactatgtgggcatgtaacaccggactcactccatgcctatccgccaccgtgcttaatcgcaccactgactactgcgttctcgtaaaattatggcccagggtcacctaccatcctcccagttacgtctataaccagtttgaaaactcctatagacataaaagagaaccagtgtccttaaccttggccttattattaggtgggctaactatgggtggcatcgccgcgggagtagggacaggaactaccgccctggtcgccacccagcagtttcagcagctccatgctgccgtacaagatgatctcaaagaggtcgaaaagtcaattactaacctagaaaagtctcttacttcgttgtctgaggttgtactgcagaatcgacgaggcctagacctgttgttcctaaaagaaggagggctgtgtgctgccctaaaagaagaatgttgtttctatgctgaccacacaggcctagtaagagatagtatggccaaattaagagagagactcactcagagacaaaaactatttgagtcgagccaaggatggttcgaaggattgtttaacagatccccctggtttaccacgttaatatccaccatcatggggcctctcattatactcctactaattctgctttttggaccctgcattcttaatcgattagtccaatttgttaaagacaggatatcagtggtccaggctctagttttgactcaacaatatcaccagctgaagcctatagagtacgagccatag

SEQ ID NO: 2 - DNA, 2009 bp, Artificial Sequence
  Description of Artificial Sequence Synthetic Clone ID 4-4
tcaacgctctcaaaatcccctaaagataagattgacccgcgggacctcctaatccccttaattctcttcctgtctctcaaaggagccagatccgcagcgcccggctccagccctcaccaggtctacaacattacctgggaagtgaccaatggggatcgggaggcagtatgggcaatatcaggcaaccaccctctgtggacttggtggccagacctcaccccagatttgtgtatgttagctctcagtgggccgccccactgggggctagaatatcaggccccctattcctcgcccccggggcccccttgttgctcagggagcagcgggaacagtgcaggctgtttcagagactgcgacgagcccttgacctccctcacccctcggtgcaacactgcctggaacagacttaagctagaccaggtaactcataaatcaagtgagggattttatgtctgccccgggccacatcgcccccgggaagccaagtcctgtggaggtccagattccttctactgtgcctcttggggctgcgagacaaccggtagagtatactggaagccctcctcctcttgggactacatcacagtagacaacaatctcaccactaaccaggctgttaaggtatgcaaagacaataagtggtgcaatcccttggctatccggtttacaaacaccgggagacaggtcacctcatggacaactggacactattggggtctacgtctttatgtcactgggaaggacccggggcttactttcgggatcagactcaaatatcaaaatctaggacctcgggtcccaataggaccaaaccccgtcctggcagaccaactttcgttcccgctacctaatcccctacccaaacctgccaagtctccccccgcctctaattcgactcccacattgatttccccgtcccccactcccactcagcccccgccagcaggaacgggagacaggttactaaatctagtacagggagcttaccaggcactcaaccttaccaaccctgataaaactcaagagtgctggttatgcctagtgtctggacccccctattacgagggagttgcggtcctaggtacttattccaaccatacctctgccccagctaactgctccacggcctcccaacacaagttgaccctgtccgaagtgactggacggggactctgcataggaacagtcccaaaaactcaccaggccctgtgcaacactacccttaagacaggcaaagggtcttactatctagttgcccccgcaggaactatgtgggcatgtaacaccggactcactccatgcctatccgccaccgtgcttaatcgcaccacgttctcgtagaattatggcccagggtcacctaccatcctcccagttacgtctatagccagtttgaaaactcctatagacataaaagagaaccagtgtccttaaccttggccttattattaggtgggctaactatgggtggcatcgccgcgggagtagggacaggaactaccgccctggtcgccacccagcagtttcagcagctccatgctgccgtacaagatgatctcaaagaggtcgaaaagtcaattactaacctagaaaagtctcttacttcgttgtctgaggttgtactgcagaatcgacgaggcctagacctgttgttcctaaaagaaggaggactgtgtgctgccctaaaagaagaatgttgtttctatgctgaccacacaggcctagtaagagatagtatggccaaattaagagagagactcactcagagacaaaaactatttgagtcgagccaaggatggttcgaaggattgtttaacagatccccctggtttaccacgttaatatccaccatcatggggcctctcattatactcctactaattctgctttttggaccctgcattcttaatcgattagtccaatttgttaaagacaggatatcagtggtccaggctctagttttgactcaacaatatcaccagctgaagcctatagagtacgagccatag

SEQ ID NO: 3 - DNA, 2028 bp, Artificial Sequence
  Description of Artificial Sequence Synthetic Clone ID 4-7
atggcgtgttcaacgctctcaaaatcccctaaagataagattgacccgcgggacctcctaatccccttaattctcttcctgtctctcaaaggggccagatccgcagcacccggctccagccctcaccaggtctacaacattacctgggaagtgaccaatggggatcaggaggcagtatgggcaatatcaggcaaccaccctctgtggacttggtggccagacctcaccccagatttgtgtatgttagctctcagtgggccgccccactgggggctagaatatcaggccccctattcctcgcccccggggcccccttgttgctcagggagcagcgggaacagtgcaggctgtttcagagactgcgacgagcccttgacctccctcacccctcggtgcaacactgcctggaacagacttaagctagaccaggtaactcatagatcaagtgagggattttatgtctgccccgggccacatcgcccccgggaagccaagtcctgtggaggtccagattccttctactgtgcctcttggggctgcgagacaaccggtagagtatactggaagccctcctcctcttgggactacatcacagtagacaacaatctcaccactaaccaggctgctcaggtatgcaaagacaataagtggtgcaatcccttggctatccggtttacaaacgccgggagacaggtcacctcatggacaactggacactattggggtctacgtctttatgtcactgggaaggacccggggcttactttcgggatcagactcaaatatcaaaatctaggacctcgggtcccaataggaccaaaccccgtcctggcagaccaactttcgttcccgctacctaatcccctacccaaacctgccaagtctccccccgcctctaattcgactcccacattgatttccccgtcccccactcccactcagcccccgccagcaggaacgggagacaggttactaaatctagtacagggagcttaccaggcactcaaccttaccaaccctgataaaactcaagagtgctggttatgcctagtgtctggacccccctattacgagggagttgcggtcctaggtacttattccaaccatacctctgccccagctaactgctccacggcctcccaacacaagttgaccctgtccgaagtgactggacggggactctgcataggaacagtcccaaaaactcaccaggccctgtgcaacactacccttaaggcaggcaaagggtcttactatctagttgcccccgcaggaactatgtgggcatgtaacaccggactcactccatgcctatccgccaccgtgcttaatcgcaccactgactactgcgttctcgtagaattatggcccagggtcacctaccatcctcccagttacgtctatagccagtttgaaaactcctatagacataaaagagaaccagtgtccttaaccttggccttattattaggtgggctaactatgggtggcatcgccgcgggagtagggacaggaactaccgccctggtcgccacccagcagtttcagcagctccatgctgccgtacaagatgatctcaaagaggtcgaaaagtcaattactaacctagaaaagtctcttacttcgttgtctgaggttgtactgcagaatcgacgaggcctagacctgttgttcctaaaagaaggaggactgtgtgctgccctaaaagaagaatgttgtttctatgctgaccacacaggcctagtaagagatagtatggccaaattaagagagagactcactcagagacaaaaactatttgagtcgagccaaggatggttcgaaggattgtttaacagatccccctggtttaccacgttaatatccaccatcatggggcctctcattatactcctactaattctgctttttggaccctgcattcttaatcgattagtccaatttgttaaagacaggatatcagtggtccaggctctagttttgactcaacaatatcaccagctgaagcctatagagtacgagccatag

SEQ ID NO: 4 - protein, 672 aa, Artificial Sequence
  Description of Artificial Sequence Synthetic Clone ID 2B-17
STLSKSPKDKIDPRDLLIPLILFLSLKGARSAAPGSSPHQVYNITWEVTNGDQEAVWAISGNHPLWTWWPDLTPDLCMLALSGPPHWGLEYQAPYSSPPGPPCCSGSSGNSAGCFRDCDEPLTSLTPRCNTAWNRLKLDQVTHKSSEGFYVCPGPHRPREAKSCGGPDSFYCASWGCETTGRVYWKPSSSWDYITVDNNLTTNQAVKVCKDNKWCNPLAIRFTNTGRQVTSWTTGHYWGLRLYVTGKDPGLTFGIRLKYQNLGPRVPIGPNPVLADQLSFPLPNPQPKPAKSPPASNSTPTLISPSPTPTQPPPAGTGDRLLNLVQGAYQALNLTNPDKTQECWLCLVSGPPYYEGVAVLGTYSNHTSAPANCSTASQHKLTLSEVTGRGLCIGTVPKTHQALCNTTLKTGKGSYYLVAPAGTMWACNTGLTPCLSATVLNRTTDYCVLVKLWPRVTYHPPSYVYNQFENSYRHKREPVSLTLALLLGGLTMGGIAAGVGTGTTALVATQQFQQLHAAVQDDLKEVEKSITNLEKSLTSLSEVVLQNRRGLDLLFLKEGGLCAALKEECCFYADHTGLVRDSMAKLRERLTQRQKLFESSQGWFEGLFNRSPWFTTLISTIMGPLIILLLILLFGPCILNRLVQFVKDRISVVQALVLTQQYHQLKPIEYEP

SEQ ID NO: 5 - protein, 672 aa, Artificial Sequence
  Description of Artificial Sequence Synthetic Clone ID 4-4
STLSKSPKDKIDPRDLLIPLILFLSLKGARSAAPGSSPHQVYNITWEVTNGDREAVWAISGNHPLWTWWPDLTPDLCMLALSGPPHWGLEYQAPYSSPPGPPCCSGSSGNSAGCFRDCDEPLTSLTPRCNTAWNRLKLDQVTHKSSEGFYVCPGPHRPREAKSCGGPDSFYCASWGCETTGRVYWKPSSSWDYITVDNNLTTNQAVKVCKDNKWCNPLAIRFTNTGRQVTSWTTGHYWGLRLYVTGKDPGLTFGIRLKYQNLGPRVPIGPNPVLADQLSFPLPNPLPKPAKSPPASNSTPTLISPSPTPTQPPPAGTGDRLLNLVQGAYQALNLTNPDKTQECWLCLVSGPPYYEGVAVLGTYSNHTSAPANCSTASQHKLTLSEVTGRGLCIGTVPKTHQALCNTTLKTGKGSYYLVAPAGTMWACNTGLTPCLSATVLNRTTDYCVLVELWPRVTYHPPSYVYSQFENSYRHKREPVSLTLALLLGGLTMGGIAAGVGTGTTALVATQQFQQLHAAVQDDLKEVEKSITNLEKSLTSLSEVVLQNRRGLDLLFLKEGGLCAALKEECCFYADHTGLVRDSMAKLRERLTQRQKLFESSQGWFEGLFNRSPWFTTLISTIMGPLIILLLILLFGPCILNRLVQFVKDRISVVQALVLTQQYHQLKPIEYEP

SEQ ID NO: 6 - protein, 675 aa, Artificial Sequence
  Description of Artificial Sequence Synthetic Clone ID 4-7
MACSTLSKSPKDKIDPRDLLIPLILFLSLKGARSAAPGSSPHQVYNITWEVTNGDQEAVWAISGNHPLWTWWPDLTPDLCMLALSGPPHWGLEYQAPYSSPPGPPCCSGSSGNSAGCFRDCDEPLTSLTPRCNTAWNRLKLDQVTHRSSEGFYVCPGPHRPREAKSCGGPDSFYCASWGCETTGRVYWKPSSSWDYITVDNNLTTNQAAQVCKDNKWCNPLAIRFTNAGRQVTSWTTGHYWGLRLYVTGKDPGLTFGIRLKYQNLGPRVPIGPNPVLADQLSFPLPNPLPKPAKSPPASNSTPTLISPSPTPTQPPPAGTGDRLLNLVQGAYQALNLTNPDKTQECWLCLVSGPPYYEGVAVLGTYSNHTSAPANCSTASQHKLTLSEVTGRGLCIGTVPKTHQALCNTTLKAGKGSYYLVAPAGTMWACNTGLTPCLSATVLNRTTDYCVLVELWPRVTYHPPSYVYSQFENSYRHKREPVSLTLALLLGGLTMGGIAAGVGTGTTALVATQQFQQLHAAVQDDLKEVEKSITNLEKSLTSLSEVVLQNRRGLDLLFLKEGGLCAALKEECCFYADHTGLVRDSMAKLRERLTQRQKLFESSQGWFEGLFNRSPWFTTLISTIMGPLIILLLILLFGPCILNRLVQFVKDRISVVQALVLTQQYHQLKPIEYEP

SEQ ID NO: 7 - protein, 675 aa, Artificial Sequence
  Description of Artificial Sequence Synthetic Clone ID 2B-13
MACSTLSKSPKDKIDPRDLLIPLILFLSLKGARSAAPGSSPHQVYNITWEVTNGDREAVWAISGNHPLWTWWPDLTPDLCMLALSGPPHWGLEYQAPYSSPPGPPCCSGSSGNSAGCFRDCDEPLTSLTPRCNTAWNRLKLDQVTHKSSEGFYVCPGPHRPREAKSCGGPDSFYCASWGCETTGRVYWEPSSSWDYITVDNNLTTNQAVKVCKDNKWCNPLAIRFTNTGRQVTSWTTGHYWGLRLYVTGKDPGLTFGIRLKYQNLGPRVPIGPNPVLADQLSFPLPNPQPKPAKSPPASNSTPTLISPSPTPTQPPPAGTGDRLLNLVQGAYQALNFTNPDKTQECWLCLVSGPPYYEGVAVLGTYSNHTSAPANCSTASQHKLTLSEVTGRGLCIGTVPKTHQALCNTTLKTGKGSYYLVAPAGTMWACNTGLTPCLSATVLNRTTDYCVLVELWPRVTYHPPSYVYSQFENSYRHKREPVSLTLALLLGGLTMGGIAAGVGTGTTALVATQQFQQLHAAVQDDLKEVEKSITNLEKSLTSLSEVVLQNRRGLDLLFLKEGGLCAALKEECCFYADHTGLVRDSMAKLRERLTQRQKLFESSQGWFEGLFNRSPWFTTLISTIMGPLIILLLILLFGPCILNRLVQFVKDRISVVQALVLTQQYHQLKPIEYEP

SEQ ID NO: 8 - protein, 675 aa, Artificial Sequence
  Description of Artificial Sequence Synthetic Clone ID 2B-8
MACSTLSKSPKDKIDPRDLLIPLILFLSLKGARSAAPGSSPHQVYNITWEVTNGDREAVWAISGNHPLWTWWPDLTPDLCMLALSGPPHWGLEYQAPYSSPPGPPCCSGSSGNSAGCFRDCDEPLTSLTPRCNTAWNRLKLDQVTHKSSEGFYVCPGPHRPREAKSCGGPDSFYCASWGCETTGRVYWKPSSSWDYITVDNNLTTNQAVKVCKDNKWCNPLAIRFTNTGRQVTSWTTGHYWGLRLYVTGKDPGLTFGIRLKYQNLGPRVPIGPNPVLADQLSFPLPNPQPRPAKSPPASNSTPTLISPSPTPTQPPPAGTGDRLLNLVQGAYQALNFTNPDKTQECWLCLVSGPPYYEGVAVLGTYSNHTSAPANCSAASQHKLTLSEVTGRGLCIGTVPKTHQALCNTTLKTGKGSYYLVAPAGTMWACNTGLTPCLSATVLNRTTDYCVLAELWPRVTYHPPSYVYSQFENSYRHKREPVSLTLALLLGGLTMGGIAAGVGTGTTALVATQQFQQLHAAVQDDLKEVEKSITNLEKSLTSLSEVVLQNRRGLDLLFLKEGGLCAALKEECCFYADHTGLVRDSMAKLRERLNQRQKLFESTQGWFEGLFNRSPWFTTLISTIMGPLIVLLMILLFGPCILNRLVQFVKDRISVVQALVLTQQYHQLKPIEYEP

SEQ ID NO: 9 - DNA, 2028 bp, Artificial Sequence
  Description of Artificial Sequence Synthetic Clone ID 2B-13
atggcgtgttcaacgctctcaaaatcccctaaagataagattgacccgcgggacctcctaatccccttaattctcttcctgtctctcaaaggagccagatccgcagcgcccggctccagccctcaccaggtctacaacattacctgggaagtgaccaatggggatcgggaggcagtatgggcaatatcaggcaaccaccctctgtggacttggtggccagacctcaccccagatttgtgtatgttagctctcagtgggccgccccactgggggctagaatatcaggccccctattcctcgcccccggggcccccttgttgctcagggagcagcgggaacagtgcaggctgtttcagagactgcgacgagcccttgacctccctcacccctcggtgcaacactgcctggaacagacttaagctagaccaggtaactcataaatcaagtgagggattttatgtctgccccgggccacatcgcccccgggaagccaagtcctgtggaggtccagattccttctactgtgcctcttggggctgcgagacaaccggtagagtatactgggagccctcctcctcttgggactacatcacagtagacaacaatctcaccactaaccaggctgttaaggtatgcaaagacaataagtggtgcaatcccttggctatccggtttacaaacaccgggagacaggtcacctcatggacaactggacactattggggtctacgtctttatgtcactgggaaggacccggggcttactttcgggatcagactcaaatatcaaaatctaggacctcgggtcccaataggaccaaaccccgtcctggcagaccaactttcgttcccgctacctaatccccaacccaaacctgccaagtctccccccgcctctaattcgactcccacattgatttccccgtcccccactcccactcagcccccgccagcaggaacgggagacaggttactaaatctagtacagggagcttaccaggcactcaactttaccaaccctgataaaactcaagagtgctggttatgcctagtgtctggacccccctattacgagggagttgcggtcctaggtacttattccaaccatacctctgccccagctaactgctccacggcctcccaacacaagttgaccctgtccgaagtgactggacggggactctgcataggaacagtcccaaaaactcaccaggccctgtgcaacactacccttaagacaggcaaagggtcttactatctagttgcccccgcaggaactatgtgggcatgtaacaccggactcactccatgcctatccgccaccgtgcttaatcgcaccactgactactgcgttctcgtagaattatggcccagggtcacctaccatcctcccagttacgtctatagccagtttgaaaactcctatagacataaaagagaaccagtgtccttaaccttggccttattattaggtgggctaactatgggtggcatcgccgcgggagtagggacaggaactaccgccctggtcgccacccagcagtttcagcagctccatgctgccgtacaagatgatctcaaagaggtcgaaaagtcaattactaacctagaaaagtctcttacttcgttgtctgaggttgtactgcagaatcgacgaggtctagacctgttgttcctaaaagaaggaggactgtgtgctgccctaaaagaagaatgttgtttctatgctgaccacacaggcctagtaagagatagtatggccaaattaagagagagactcactcagagacaaaaactatttgagtcgagccagggatggttcgaaggattgtttaacagatccccctggtttaccacgttaatatccaccatcatggggcctctcattatactcctactaattctgctttttggaccctgcattcttaatcgattagtccaatttgttaaagacaggatatcagtggtccaggctctagttttgactcaacaatatcaccagctgaagcctatagagtacgagccatag

SEQ ID NO: 10 - DNA, 2028 bp, Artificial Sequence
  Description of Artificial Sequence Synthetic Clone ID 2B-8
atggcgtgttcaacgctctcaaaatcccctaaagataagattgacccgcgggacctcctaatccccttaattctcttcctgtctctcaaaggggccagatccgcagcacccggctccagccctcaccaggtctacaacattacctgggaagtgaccaatggggatcgggaggcagtatgggcaatatcaggcaaccaccctctgtggacttggtggccagacctcaccccagatttgtgtatgttagctctcagtgggccgccccactgggggctagaatatcaggccccctattcctcgcccccggggcccccttgttgctcagggagcagcgggaacagtgcaggctgtttcagagactgcgacgagcccttgacctccctcacccctcggtgcaacactgcctggaacagacttaagctagaccaggtaactcataaatcaagtgagggattttatgtctgccccgggccacatcgcccccgggaagccaagtcctgtggaggtccagattccttctactgtgcctcttggggctgcgagacaaccggtagagtatactggaagccctcctcctcttgggactacatcacagtagacaacaatctcaccactaaccaggctgttaaggtatgcaaagacaataagtggtgcaaccccttggctatccggtttacaaacaccgggagacaggtcacctcatggacaactggacactattggggtctacgtctttatgtcactgggaaggacccggggcttactttcgggatcagactcaaatatcaaaatctaggacctcgggtcccaataggaccaaaccccgtcctggcagaccaactttcgttcccgctacctaatccccaacccagacctgccaagtctccccccgcctctaattcgactcccacattgatttccccgtcccccactcccactcagcccccgccagcaggaacgggagacaggttactaaatctagtacagggagcttaccaggcactcaactttaccaaccctgataaaactcaagagtgctggttatgcctagtgtctggacccccctattacgagggagttgcggtcctaggtacttattccaaccatacctctgccccagctaactgctccgcggcctcccaacacaagttgaccctgtccgaagtgactggacggggactctgcataggaacagtcccaaaaactcaccaggccctgtgcaacactacccttaagacaggcaaagggtcttactatctagttgcccccgcaggaactatgtgggcatgtaacaccggactcactccatgcctatccgccaccgtgcttaatcgcaccactgactactgcgttctcgcagaattatggcccagggtcacctaccatcctcccagttacgtctatagccagtttgaaaactcctatagacataaaagagaaccagtgtccttaaccttggccttattattaggtgggctaactatgggtggcatcgccgcgggagtagggacaggaactaccgccctggtcgccacccagcagtttcagcagctccatgctgccgtacaagatgatctcaaagaggtcgaaaagtcaattactaacctagaaaagtctcttacttcgttgtctgaggttgtactgcagaatcgacgaggcctagacctgttgttcctaaaagaaggaggactgtgtgctgccctaaaagaagaatgttgcttctatgcggaccacacaggactagtgagagacagcatggccaaattgagagagaggcttaatcagagacagaaactgtttgagtcaactcaaggatggtttgagggactgtttaacagatccccttggtttaccaccttgatatctaccattatgggacccctcattgtactcctaatgattttgctcttcggaccctgcattcttaatcgattagtccaatttgttaaagacaggatatcagtggtccaggctctagttttgactcaacaatatcaccagctgaagcctatagagtacgagccatag

SEQ ID NO: 11 - protein, 676 aa, Friend murine leukemia virus
MACSTLSKSPKDKIDPRDLLIPLILFLSLKGARSAAPGSSPHQVYNITWEVTNGDREAVWAISGNHPLWTWWPDLTPDLCMLALSGPPHWGLEYQAPYSSPPGPPCCSGSSGNSAGCSRDCNEPLTSLTPRCNTAWNRLKLDQVTHKSSEGFYVCPGSHRPREAKSCGGPDSFYCASWGCETTGRVYWKPSSSWDYITVDNNLTTNQAVQVCKDNKWCNPLAIRFTNAGRQVTSWTTGHSWGLRLYVTGKDPGLTFGIRLKYQNLGPRVPIGPNPVLADQLSFPLPNPQPKPAKSPPASNSTPTLISPSPTPTQPPPAGTGDRLLNLVQGAYQALNLTNPDKTQECWLCLVSGPPYYEGVAVLGTYSNHTSAPANCSAASQHKLTLSEVTGRGLCIGTVPKTHQALCNTTLKTGKGSYYLVAPAGTMWACNTGLTPCLSATVLNRTTDYCVLVELWPRVTYHPPSYVYSQFENSYRHKREPVSLTLALLLGGLTMGGIAAGVGTGTTALVATQQFQQLHAAVQDDLKEVEKSITNLEKSLTSLSEVVLQNRRGLDLLFLKEGGLCAALKEECCFYADHTGLVRDSMAKLRERLTQRQKLFESSQGWFEGLFNRSPWFTTLISTIMGPLIILLLILLFGPCILNRLVQFVKDRISVVQALVLTQQYHQLKPLEYEPQ

SEQ ID NO: 12 - protein, 25 aa, Artificial Sequence
  Description of Artificial Sequence Synthetic peptide
VCPGPHRPREAKSCGGPDSFYCASW

SEQ ID NO: 13 - protein, 25 aa, Artificial Sequence
  Description of Artificial Sequence Synthetic peptide
LCPGPHKPREAKSCGGPESFYCTSW

SEQ ID NO: 14 - protein, 25 aa, Artificial Sequence
  Description of Artificial Sequence Synthetic peptide
VCPGPHRPRDSKSCGGPDSYYCATW

(19) United States
(12) Patent Application Publication
(10) Pub. No.: US 2002/0137889 A1
(43) Pub. Date: Sep. 26, 2002

(54) Stress resistant retroviruses

(76) Inventors: Nay Wei Soong, San Jose, CA (US); Willem P.C. Stemmer, Los Gatos, CA (US); Sharon K. Powell, Alameda, CA (US); Edward Otto, Falls Church, VA (US)

Correspondence Address: LAW OFFICES OF JONATHAN ALAN QUINE, P O BOX 458, ALAMEDA, CA, 94501

(21) Appl. No.: 09/954,983

(22) Filed: Sep. 17, 2001

Related U.S. Application Data
(60) Provisional application No. 60/233,398, filed on Sep. 18, 2000.

(51) Int. Cl. C12N 7/01; C12P 21/02; C07H 21/04; C07K 14/15; C12N 5/06

(52) U.S. Cl. 530/350; 435/235.1; 435/69.1; 435/325; 536/23.72

(57) ABSTRACT

Stress and/or shear resistant retrovirus envelope protein polypeptides and nucleic acids encoding such polypeptides, as well as fragments of such nucleic acids and polypeptides and compositions thereof, are provided. Retroviruses incorporating such polypeptides and methods of using stress resistant retrovirus envelope protein polypeptides and corresponding nucleic acids are also described.

### CROSS-REFERENCE TO RELATED APPLICATIONS

This application claims priority to and benefit of U.S. Provisional Patent Application Ser. No. 60/233,398 filed Sep. 18, 2000, the disclosure of which is incorporated herein by reference in its entirety for all purposes.

### COPYRIGHT NOTIFICATION PURSUANT TO 37 C.F.R. § 1.71(E)

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

Retroviral vectors for in vivo and ex vivo mammal (e.g., human) gene therapy require highly purified and high titer preparations. It is estimated that when using conventional producer cell culture systems, downstream processes must provide product concentration of up to 100 fold (Braas et al. (1996) Bioseparation 6:211). On a manufacturing scale, robust and consistent purification and concentration schemes for retroviral vectors that satisfy potency, quality and cost considerations are of paramount importance. This has been complicated by the substantial sensitivity of murine retrovirus based vectors to physical processes commonly used for concentration, resulting in the inability to manufacture efficiently retroviral vector stocks of high titer (Friedmann et al. (1995) Nat Med 6:275). The instability of retroviral vectors during concentration methods, such as ultracentrifugation, appears to be linked to properties of the viral envelope protein, since substitution or “pseudotyping” of the retroviral envelope protein with the Vesicular Stomatitis Virus G protein (VSV-G) allows for the concentration of retroviral vectors by ultracentrifugation ( Friedmann, ibid; Burns et al. (1993) Proc. Nat'l Acad. Sci. USA 90:8033). However, the cytotoxicity of G protein makes the production of such pseudotyped vectors from stable packaging cell lines technically difficult, and also broadens the host range of the vector, which may be undesirable for some applications.

The envelope protein of murine type C retroviruses is composed of two subunits, SU (gp 70) and TM (plSE), which mediate binding to cellular receptors and fusion with the plasma membrane, respectively (Green et al. (1981) Proc. Nat'l Acad. Sci. USA 78:6023; Hunter et al. (1990) Curr. Top. Microbiol. Immunol. 157:187). Several studies suggest that a conformational change occurring after release of the SU region triggers membrane fusion (Fass et al. (1996) Curr. Biol. 5:1377; Fass et al. (1996) Nat. Struct. Bio. 3:465; Bae et al. (1997) J. Virol. 71:2092). The SU and TM subunits are associated by a labile disulfide bond; instability of retroviruses during purification is thought to be due to loss of the SU domain (Aboud et al. (1982) Archives of Virology 71:185; Pinter et al. (1978) Virology 91:345). This is consistent with the decreased amounts of SU observed in preparations of ultracentrifuged retrovirus (McGrath et al. (1978) J. Virol. 25:923). Viruses purified by ultrafiltration methods have also been reported to have a loss of infectivity but not reverse transcriptase activity during processing under high pressure, suggesting that the viral cores remain intact but lose surface proteins necessary for infectivity (Paul et al. (1993) Hum. Gene Ther. 4:609).

Accordingly, it would be advantageous to develop retroviruses that are resistant to stress or shear forces experienced during manufacturing procedures, such as during purification and concentration procedures. The present invention fulfills these and other needs.

### SUMMARY OF THE INVENTION

The invention provides novel retrovirus envelope protein polypeptides, nucleic acids encoding the polypeptides, retroviruses comprising the polypeptides and uses therefor.

In one aspect, the invention includes an isolated, recombinant, or modified retrovirus envelope protein nucleic acids. Included in the invention are isolated, modified, or recombinant nucleic acid sequences comprising a polynucleotide sequence selected from the group consisting of: (a) nucleic acid sequence comprising any one of SEQ ID NOS:1-3 and SEQ ID NOS:9-10, or complementary polynucleotide sequence thereof; (b) a polynucleotide sequence encoding a polypeptide selected from SEQ ID NOS:4-8, or a complementary polynucleotide sequence thereof; (c) a polynucleotide sequence that hybridizes under highly stringent conditions over substantially the entire length of the preceding polynucleotide sequences (a) or (b); (d) a polynucleotide sequence comprising a nucleotide fragment of (a), (b), or (c), wherein said fragment encodes all or part of a stress or shear resistant retrovirus envelope protein; and (e) a polynucleotide sequence which, but for the degeneracy of the genetic code, hybridizes under highly stringent conditions over substantially the entire length of polynucleotide sequence (a), (b), (c), or (d).

In some embodiments, the encoded polypeptide comprises an amino acid sequence selected from any of the group of SEQ ID NOS:4-8; and the nucleic acid comprises a polynucleotide sequence selected from any of the group of SEQ ID NOS:1-3 and/or SEQ ID NOS:9-10.

The invention also includes an isolated, modified or recombinant nucleic acid comprising a polynucleotide sequence encoding a polypeptide, where the polypeptide comprises an amino acid sequence comprising at least 20 contiguous amino acids of any one of SEQ ID NOS:4-8. In various embodiments, the encoded polypeptide comprises at least 50, at least 100, at least 200, at least 300, at least 400, at least 500, at least 600, at least 615, at least 620, at least 625, at least 650, at least 660, at least 665, at least 670, at least 671, at least 672, or at least 675 contiguous amino acid residues of any one of SEQ ID NOS:4-8. In another embodiment, the encoded polypeptide is about 672 or 675 amino acids in length.

In some embodiments, the polypeptide encoded by any isolated or recombinant nucleic acid of the invention described above confers stress (and/or shear) resistance on a retrovirus of which it is an envelope protein. In some embodiments, the retrovirus comprising said stress or shear resistant envelope protein polypeptide encoded by a nucleic acid of the invention is capable of withstanding ultracentrifugation at a force of least about or in excess of 120,000 ×gravity (g) for at least about 20, 30, 40, 50, 60, 68, 70, 80, or 90 minutes or more, such that the retrovirus or supernatant or solution comprising the retrovirus has, retains, or preserves an infectious titer level of at least about 25%, 30%, 40%, 50%, 60%, 70%, 80%, 85%, 90%, 91%, 92%, 93%, 94%,95%, 96%, 97%, 98%, 98.5%, 99%, 99.5%, or 100% of the infectious titer level of an identical retrovirus comprising said stress or shear resistant envelope protein polypeptide (or supernatant or solution comprising such retrovirus) that has not undergone such ultracentrifugation.

In another aspect, the invention provides an isolated, modified, or recombinant nucleic acid comprising a polynucleotide sequence encoding a polypeptide, the polypeptide comprising an amino acid sequence comprising at least 500 contiguous amino acids of any one of SEQ ID NOS:4-8. In some aspects, such nucleic acid encodes a polypeptide having resistant to a stress or shear force experienced during ultracentrifugation for a selected time period, such as the force resulting at 120,000 x g or at least 30, 60, 65, 68, 70, 75, 80, 85, 90 or more minutes. In some aspects, a retrovirus comprising such stress or shear resistant retrovirus polypeptide as an envelope protein (or composition comprising such retrovirus) is capable of maintaining an infectious titer level following such ultracentrifugation procedures that is at least about 30%, 40%, or 50% of an infectious titer level of an identical retrovirus comprising such stress or shear resistant retrovirus polypeptide as an envelope protein (or composition of such retrovirus) that has not undergone such ultracentrifugation procedure.

The invention also includes an isolated, modified or recombinant nucleic acid that encodes a stress or shear resistant retrovirus envelope polypeptide capable of, withstanding ultracentrifugation at a force of at least about 120,000×g for at least about 30, 45, 60, 65, 68, 70, 75, 80, 85, 90 or more minutes.

The invention also provides a nucleic acid that encodes a stress or shear resistant retrovirus envelope polypeptide, said polypeptide has at least about 95% amino acid sequence identity with an amino acid sequence of at least one of SEQ ID NOS:4-6.

The invention also includes a cell comprising any nucleic acid of the invention described above, or that expresses any polypeptide of the invention described herein. In one embodiment, the cell expresses a polypeptide encoded by the nucleic acid.

The invention also includes a vector comprising any nucleic acid described above. The vector can comprise a plasmid, a cosmid, a phage, or a virus, including a retrovirus; the vector can be, e.g., an expression vector, a cloning vector, a packaging vector, an integration vector, or the like. The invention also includes a cell transduced by the vector. Cells infected with retroviruses incorporating the nucleic acids described above, and/or including the envelope protein polypeptides, e.g., encoded by the nucleic acids, described above are a feature of the invention.

The invention also includes compositions comprising any nucleic acid, described above, and an excipient, preferably a pharmaceutically acceptable excipient. Cells and transgenic animals that include any polypeptide or nucleic acid above, e.g., produced by transduction of the vector, are a feature of the invention.

The invention also includes compositions comprising two or more nucleic acids described above. The composition may comprise a library of nucleic acids of the invention, wherein each said nucleic acid encodes a stress or shear resistant retrovirus envelope protein polypeptide, and said library contains at least 2, 10, 20, 50, or more such nucleic acids.

In another aspect, the invention includes a recombinant polypeptide encoded by any nucleic acid described above. In one embodiment, the polypeptide may comprise a sequence selected from any of SEQ ID NOS:4-8.

The invention also includes a polypeptide comprising at least 100 contiguous amino acids of a protein encoded by a polynucleotide sequence, the polynucleotide sequence selected from the group consisting of: (a) SEQ ID NO:1 to SEQ ID NO:3 and/or SEQ ID NOS:9-10; (b) a polynucleotide sequence that encodes a polypeptide selected from SEQ ID NOS:4-8; and (c) a complementary sequence of a polynucleotide sequence that hybridizes under highly stringent conditions over substantially the entire length of polynucleoide sequence (a) or (b). In various embodiments, the polypeptide comprises at least about 200, 300, 400, 500, 550, 600, 615, 620, 650, 655, 660, 665, 670, 671, 672, or 675 contiguous amino acids of the encoded protein. In some embodiments, the polypeptide comprises a retrovirus envelope protein of 672 amino acids. In other embodiments, the polypeptide comprises a retrovirus envelope protein of 675 amino acids. Longer polypeptides, e.g., which comprise purification tags or the like, are also contemplated.

In some embodiments, such a polypeptide comprises a stress and/or shear resistant retrovirus envelope protein. In some such embodiments, said stress and/or shear resistant retrovirus envelope protein is capable is withstanding ultracentrifugation at a force of at least about 120,000×g for at least about 60 minutes such that a retrovirus incorporating said shear resistant retrovirus envelope protein has an infectious titer level that is at least about 25%, 30%, 40%, 50%, 60%, 70%, 80%, 85%, 90%, 91%, 92%, 93%, 94%, 95%, 96%, 97%, 98%, 99% or 100% of the infectious titer level of an identical retrovirus incorporating said shear resistant retrovirus envelope protein that is not subjected to such ultracentrifugation procedures.

In one embodiment, the invention provides an isolated, modified or recombinant polypeptide comprising an amino acid sequence comprising at least 650 contiguous amino acids of any of SEQ ID NOS:4-6. In another embodiment, the invention includes a stress and/or shear resistant retrovirus comprising an envelope protein selected from SEQ ID NOS:4-6. In another embodiment, the invention provides a retrovirus comprising an envelope protein selected from SEQ ID NOS:7-8.

In other embodiments, any polypeptide described above may further include a secretionflocalization sequence, e.g., a signal sequence, an organelle targeting sequence, a membrane localization sequence, and the like. Any polypeptide described above may further include a sequence that facilitates purification, e.g., an epitope tag (such as, a FLAG epitope), a polyhistidine tag, a GST fusion, and the like. The polypeptide optionally includes a methionine at the N-terminus. Any polypeptide described above optionally includes one or more modified amino acids, such as a glycosylated amino acid, a PEG-ylated amino acid, a farnesylated amino acid, an acetylated amino acid, a biotinylated amino acid, a carboxylated amino acid, a phosphorylated amino acid, an acylated amino acid, or the like.

The invention also includes compositions comprising any polypeptide, or retrovirus comprising a polypeptide, described above in an excipient, preferably a pharmaceutically acceptable excipient.

Methods for producing the polypeptides of the invention are also included. One such method comprises introducing into a population of cells any nucleic acid of the invention, as described above, said nucleic acid being operatively linked to a regulatory sequence effective to produce the encoded polypeptide, and culturing the cells in a culture medium to produce the polypeptide. The polypeptide is then isolated from the cells or from the culture medium. In some embodiments of the invention, the nucleic acid is part of a vector, such as a recombinant expression vector.

In addition, the invention provides methods of producing a polypeptide comprising infecting a population of cells with a retrovirus comprising any nucleic acid of the invention, as described above, and culturing the cells in a culture medium to produce a polypeptide encoded by the nucleic acid. The polypeptide can then be isolated from the cells or culture medium.

Also provided by the invention are methods of producing a stress and/or shear resistant retrovirus which comprises infecting a population of cells with a retrovirus comprising any nucleic acid of the invention, as described above, and culturing the cells in a culture medium to produce a plurality of amplified retroviruses comprising the polypeptide encoded by the nucleic acid. The plurality of amplified retroviruses is isolated from the cells or culture medium.

The invention also includes methods for gene therapy by contacting target cells or organism with a retrovirus of the invention, comprising a therapeutic or prophylactic gene construct of the invention, in vitro, ex vivo, or in vivo.

Also included in the present invention are stress and/or shear resistant retroviruses or replicative retroviruses that comprise a stress and/or shear resistant retrovirus envelope protein polypeptide of the invention as an envelope protein.

In another aspect, the invention provides a nucleic acid that comprises a unique subsequence in a nucleic acid selected from any of the group of SEQ ID NO:1 to SEQ ID NO:3 and/or SEQ ID NOS:9-10, wherein the unique subsequence is unique as compared to a nucleic acid corresponding to a naturally occurring or known nucleic acid sequence, such as a naturally occurring or known retroviral or MLV nucleic acid sequence, including an MLV nucleic acid sequence present in GenBank. In yet another aspect, the invention provides a polypeptide that comprises a unique amino acid subsequence in a polypeptide selected from any of the group of SEQ ID NOS:4-8, wherein the unique amino acid subsequence is unique as compared to an amino acid subsequence polypeptide corresponding to a naturally occurring or known envelope protein, such as a naturally occurring or known retroviral or MLV envelope protein, including a n MLV envelope protein present in GenBank.

In another aspect, the invention provides a target nucleic acid that hybridizes under highly stringent conditions to a unique coding oligonucleotide that encodes a unique amino acid subsequence in a polypeptide selected from any of SEQ ID NOS:4-8, wherein the unique amino acid subsequence is unique as compared to an amino acid subsequence of a naturally occurring or known retroviral or MLV envelope protein, including an MLV envelope protein present in GenBank. In some such aspects, the stringency conditions are selected such that a perfectly complementary oligonucleotide to the unique coding oligonucleotide hybridizes to the unique coding oligonucleotide with at least a 5x higher signal to noise ratio than for hybridization of the perfectly complementary oligonucleotide to a control nucleic acid corresponding to a naturally occurring or known retroviral or MLV envelope protein, including an MLV envelope protein present in GenBank.

In addition, the invention provides a stress or shear resistant retrovirus envelope polypeptide comprising an amino acid sequence that has at least about 90%, 91%, 92%, 93%, 94%, 95%, 96%, 97%, 98%, or 99% amino acid sequence identity with an amino acid sequence of at least one of SEQ ID NOS:4-6. In some such embodiments, the stress or shear resistant retrovirus envelope polypeptide is not the polypeptide represented by SEQ ID NO:7 or SEQ ID NO:8. In other such embodiments, the stress or shear resistant retrovirus envelope polypeptide is capable of withstanding ultracentrifugation at a force of at least about 120,000×g for at least about 30, 40, 50, 60, 68 or 80 minutes.

In another aspect, the invention provides a polypeptide that comprises an amino acid sequence encoded by a coding polynucleotide sequence, the coding polynucleotide sequence selected from the group of: (a) a polynucleotide sequence selected from at least one of SEQ ID NOS:1-3 and SEQ ID NOS:9-10, or a complementary nucleic acid sequence thereof; (b) a polynucleotide sequence that encodes a polypeptide selected from any of SEQ ID NOS:4-8; (c) a polynucleotide sequence which hybridizes under stringent conditions over substantially the entire length of a polynucleotide sequence (a) or (b); (d) a polynucleotide sequence encoding a polypeptide comprising an amino acid sequence that is substantially identical over at least about 550 contiguous amino acid residues of any one of SEQ ID NOS:4-8, provided said amino acid sequence is not a protein sequence encoded by a nucleotide sequence represented by any of GenBank Accession Nos. Z35109, K02714, Y13893, D88386, Z11128, M93134, X02794, AF288940, U94692, AF030173, S80869, S77017, S77015, S77012, J01998, AF169256, and AH000833; and (e) a polynucleotide sequence encoding a stress or shear resistant retrovirus envelope polypeptide, which polynucleotide sequence has at least about 95% identity to at least one polynucleotide sequence of (a), (b), (c), or (d).

In yet another embodiment, the invention provides an isolated, modified or recombinant variant polypeptide of a parent polypeptide, the variant polypeptide comprising at least one substitution from the group of Q454K and S469N, wherein the parent polypeptide comprises amino acid residue 4 to amino acid residue 615 of the known MLV sequence shown in SEQ ID NO:11 (GenBank Accession No. CAA84492). Some such isolated, modified or recombinant variant polypeptides further comprising at least one additional substitution selected from the group of R56Q, K147R, V209A, Q210K, A228T, Q289L, A378T, and T413A. For some such isolated, modified or recombinant variant polypeptides, the parent polypeptide comprises amino acid residue 4 to amino acid residue 671 of SEQ ID NO:11.

Also included is a stress or shear resistant retrovirus comprising an envelope protein polypeptide selected from any of SEQ ID NOS:4-6. In some aspects, retrovirus of the invention incorporates a polypeptide selected from any of SEQ ID NOS:4-6 as an envelope protein. Such stress or shear retroviruses maintain infectivity and high titer during production. Also provided are cells transduced with vectors comprising stress or shear resistant retroviruses genes. Also included is a retrovirus comprising an envelope protein polypeptide selected from any of SEQ ID NOS:7-8.

These and other objects and features of the invention will become more fully apparent when the following detailed description is read in conjunction with the accompanying figures.

### BRIEF DESCRIPTION OF THE FIGURES

FIG. 1 is a line graph illustrating residual titer and reverse transcriptase (RT) activity following ultracentrifugation of retroviral vector that is representative of retroviruses. A β-galactosidase (β-gal) encoding vector was subjected to ultracentrifugation for times ranging between 0 and 70 minutes. Aliquots of supernatant from each time point were used to infect 3T3 cells. The transduction efficiency for these samples was determined used a fluorescent substrates for β-gal detected by flow cytometry according to standard methods. To ensure that reductions in infectivity were not due to loss in recovery of virus, a reverse transcriptase (RT) assay was run on the same samples. At a time point equaling about 68 minutes, transduction was reduced about 95% to about 98%, whereas at this time point there was only a modest loss of RT activity.

FIG. 2 is a bar graph showing that representative non-stress or non-shear resistant (“parental”) ecotropic replication competent retroviruses (RCRs) are sensitive to ultracentrifugation. Stocks of RCRs were generated by transient transduction of 293 cells. These supernatants were then subjected to centrifugation at 120,000×g for about 65 or 68 minutes. The titers of treated and uncentrifuged control virus (gray bars) were compared in a limiting-dilution vector rescue assay. While the titers of the parental stocks varied, all five parentals from which titer was obtained exhibited a 30- to 100-foled loss in titer after about 68 minutes of centrifugation (black bars).

FIG. 3 is a bar graph depicting the relative stress (and/or shear) resistance of representative non-stress or non-shear resistant (“parental” or control) viruses and cloned recombinant or modified viruses. Viruses cloned from the population surviving the selection were titered by limiting-dilution vector rescue assay. The titer of centrifuged samples is shown as a percentage of the titer of the noncentrifuged control virus. Three of five modified clones derived from parental retroviruses were resistant to centrifugation up to about 80 minutes. Parental viruses run in the same experiment all showed a 30- to 100-fold drop in titer after centrifugation.

FIG. 4 shows an amino acid sequence alignment of the amino acid sequences of SEQ ID NO:4 (clone 2B-17), SEQ ID NO:5 (clone 4-4), SEQ ID NO:6 (clone 4-7), SEQ ID NO:7 (clone 2B-13) and SEQ ID NO:8 (2B-8). A retrovirus incorporating a polypeptide comprising the amino acid sequence of clone 2B-17, 44, or 4-7 as an envelope polypeptide is stress and/or shear resistant and capable of withstanding ultracentrifugation at 120,000×g for at least about 30, 60, 65, 68, 70, 75, 80, 85, 90 minutes or more; a retrovirus incorporating a polypeptide comprising the amino acid sequence of clone 2B-8 or 2B-13 is sensitive to ultracentrifugation under such conditions.

FIG. 5 shows an amino acid sequence alignment of the amino acid sequences of SEQ ID NO:11 (GenBank Accession No. CAA84492), SEQ ID NO:4 (clone 2B-17), SEQ ID NO:5 (clone 4-4), SEQ ID NO:6 (clone 4-7). SEQ ID NO:11 is the amino acid sequence of the viral envelope protein of the Friend murine leukemia virus shown at GenBank Accession No. CAA84492; the corresponding nucleic acid sequence for this retroviral envelope protein is shown at GenBank Accession No. Z35109.

### DETAILED DESCRIPTION OF THE INVENTION

Manufacturing of retroviral vectors for gene therapy is complicated by the sensitivity of these viruses to stress forces during purification and concentration. We have derived from several parental retrovirus envelope protein polypeptides several novel isolated or recombinant retrovirus envelope protein polypeptides having dramatically improved stabilities, including the capacity to maintain infectivity following ultracentrifugation. A retrovirus incorporating a stress and/or shear resistant envelope protein polypeptide of the invention as an envelope protein is able to resist stress and/or shearing forces, such as those produced during ultracentrifugation (e.g., hydrodynamic stress-producing or shear-producing force; stress or shearing force; or shearing strain), such that it has or is able to retain an infectious titer level that is not detectably or substantially reduced, or is only minimally or insignificantly reduced, relative to the infectious titer level of a sample of the same (e.g., identical) retrovirus incorporating a stress and/or shear resistant envelope protein polypeptide of the invention as an envelope protein that has not undergone such ultracentrifugation.

DEFINTIONS

A “polynucleotide sequence” is a nucleic acid which comprises a polymer of nucleic acid residues or nucleotides, A,C,T,U,G, etc., or naturally occurring or artificial nucleotide analogues), or a character string representing a nucleic acid, depending on context. Either the given nucleic acid or the complementary nucleic acid can be determined from any specified polynucleotide sequence.

“Nucleic acid” includes deoxyribonucleotide(s) or ribonucleotide(s) and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res 19:5081; Ohtsuka et al. (1985) J Biol Chem 260:2605-2608; Cassol et al. (1992); Rossolini et al. (1994) Mol Cell Probes 8:91-98). The term nucleic acid is used interchangeably with gene, cDNA, and mRNA encoded by a gene.

An “exogenous nucleic acid,” “exogenous DNA segment,” “heterologous sequence,” or “heterologous nucleic acid,” as used herein, is one that originates from a source foreign to the particular host cell, or, if from the same source, is modified from its original form. Thus, a heterologous gene in a host cell includes a gene that is endogenous to the particular host cell, but has been modified. Modification of a heterologous sequence in the applications described herein typically occurs through the use of recursive sequence recombination. The terms refer to a DNA segment which is foreign or heterologous to the cell, or homologous to the cell but in a position within the host cell nucleic acid in which the element is not ordinarily found. Exogenous DNA segments are expressed to yield exogenous polypeptides.

A “polypeptide sequence” is a polymer of amino acid residues (a protein, polypeptide, etc., comprising amino acid residues) or a character string representing an amino acid polymer, depending on context. Given the degeneracy of the genetic code, one or more nucleic acids, or the complementary nucleic acids thereof, that encode a specific polypeptide sequence can be determined from the polypeptide sequence.

Similarly, an “amino acid sequence” is a polymer of amino acid residues (a protein, polypeptide, etc.) or a character string representing an amino acid polymer, depending on context. Either the given nucleic acid that encodes an amino acid sequence, or the complementary nucleic acid thereof, can be determined from any specified amino acid sequence.

A nucleic acid, polypeptide, protein, or other component or object species is “isolated” when it is partially or completely separated from components with which it is normally associated (other proteins, nucleic acids, cells, synthetic reagents, etc.) or recovered from a component of its natural environment such that the nucleic acid, protein, other component, or object species is the predominant species present (ie., on a molar basis it is more abundant than any other individual species in the composition). In preferred embodiments, the preparation consists of more than 70%, more than 80%, or preferably more than 90% of the isolated species.

A nucleic acid, polypeptide, protein, vector, cell, or the like is “recombinant” when it is artificial or engineered, or derived from an artificial or engineered nucleic acid, polypeptide, protein, vector, cell or the like. The term may indicate that the nucleic acid, vector, or cell has been modified by the introduction of a heterologous (or foreign) nucleic acid or the alteration of a native nucleic acid, or that the protein or polypeptide has been modified by the introduction of a heterologous amino acid, or that the cell is derived from a cell so modified.

Recombinant cells express nucleic acid sequences (e.g., genes) that are not found in the native (non-recombinant) form of the cell or express native nucleic actd sequences (e.g., genes) that would be abnormally expressed under-expressed, or not expressed at all. A recombinant cell may be a cell that replicates a heterologous nucleic acid, or expresses a peptide or protein encoded by a heterologous nucleic acid. Recombinant cells may contain genes that are not found within the native (non-recombinant) form of the cell. Recombinant cells may also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

In one aspect, a “substantially pure” or “isolated” nucleic acid (RNA or DNA) or polypeptide or composition also means where the object species (e.g., nucleic acid or polypeptide) comprises at least about 50, 60, to 70 percent (on a molar basis) of all macromolecular species present. A substantially pure or isolated composition can also comprise more than about 80 to 90 percent by weight of all macromolecular species present in the composition. An isolated object species can also be purified to essential homogeneity (contaminant species cannot be detected in the composition by conventional detection methods) wherein the composition consists essentially of derivatives of a single macromolecular species. The term “purified” generally denotes that a nucleic acid, polypeptide, or protein gives rise to essentially one band in an electrophoretic gel. It typically means that the nucleic acid, polypeptide, or protein is at least about 50% pure, 60% pure, 70% pure, 75% pure, more preferably at least about 85% pure, and most preferably at least about 99% pure.

The term “isolated nucleic acid” may refer to a nucleic acid (e.g., DNA or RNA) that is not immediately contiguous with both of the coding sequences with which it is immediately contiguous (i.e., one at the 5′ and one at the 3′ end) in the naturally occurring genome of the organism from which the nucleic acid of the invention is derived. Thus, this term includes, e.g., a cDNA or a genomic DNA fragment produced by polymerase chain reaction (PCR) or restriction endonuclease treatment, whether such cDNA or genomic DNA fragment is incorporated into a vector, integrated into the genome of the same or a different species than the organism, including, e.g., a virus, from which it was originally derived, linked to an additional coding sequence to form a hybrid gene encoding a chimeric polypeptide, or independent of any other DNA sequences. The DNA may be double-stranded or single-stranded, sense or antisense.

A “subsequence” or “fragment” is any portion of an entire sequence (e.g., nucleic acid or amino acid sequence), up to and including the complete sequence. In some aspects, a fragment may comprise at least about 20, 50, 100, 200, 300, 400, 500, 600, 615, 620, 625, 650, 660, 670, 672, or 675 amino acids of a polypeptide sequence. In other aspects, a fragment may comprise at least about 100, 200, 300, 400, 500, 600, 800, 1000, 1200, 1500, 1600, 1700, 1800, 1850, 1900, 1950, 1975, 2000, or 2025 nucleotides of a nucleic acid sequence.

Numbering of a given amino acid or nucleotide polymer “corresponds to numbering” of a selected amino acid polymer or nucleic acid when the position of any given polymer component (amino acid residue, incorporated nucleotide, etc.) is designated by reference to the same residue position in the selected amino acid or nucleotide, rather than by the actual position of the component in the given polymer.

The term “wild-type” in reference to a nucleic acid sequence or amino acid sequence means that the nucleic acid sequence or amino acid sequence does not comprise any mutations. A “wild-type” polypeptide means that the polypeptide will be active at a level of activity found in nature and will comprise the amino acid sequence found in nature.

The term “related” in reference to a nucleotide sequence or polypeptide sequence means that regions or areas of the nucleotide sequence or polypeptide sequence, respectively, are identical and regions or areas of the nucleotide sequence or polypeptide sequence are heterologous.

The term “chimeric polynucleotide” means that the polynucleotide comprises regions which are wild-type and regions that are mutated. It may also mean that the polynucleotide comprises wild-type regions from one polynucleotide and wild-type regions from another related polynucleotide. The term “chimeric polypeptide” means that the polypeptide comprises regions which are wild-type and regions that are mutated; it may also mean that the polypeptide comprises wild-type regions from one polypeptide and wild-type regions from another related polypeptide.

A vector is a composition for facilitating cell transduction by a selected nucleic acid, or expression of the nucleic acid in the cell. Vectors include, e.g., plasmids, cosmids, viruses, YACs, bacteria, poly-lysine, etc.

A “recombinant expression cassette” or simply an “expression cassette” is a nucleic acid construct, generated recombinantly or synthetically, with nucleic acid elements that are capable of effecting expression of a structural gene in hosts compatible with such elements. Expression cassettes include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

“Substantially an entire length of a polynucleotide or amino acid sequence” refers to at least 60%, generally at least 70%, generally at least 80% or 85%, or typically at least 90%, 91%, 92%, 93%, 94%, 95%, 96%, 96.5%, 97%, 97.5%, 98%, 98.5%, 99%, 99.5% or more of a nucleotide or amino acid sequence.

As used herein, “naturally occurring” as applied to an object refers to the fact that it can be found in nature. For example, a polypeptide or polynucleotide sequence that is present in an organism, including, e.g., a virus, that can be isolated from a source in nature and that has not been intentionally modified by man in the laboratory is naturally occurring.

The term “homology” generally refers to the degree of similarity between two or more structures. The term “homologous sequences” refers to regions in macromolecules that have a similar order of monomers. When used in relation to nucleic acid sequences, the term “homology” refers to the degree of similarity between two or more nucleic acid sequences (e.g., genes) or fragments thereof. Typically, the degree of similarity between two or more nucleic acid sequences refers to the degree of similarity of the composition, order, or arrangement of two or more nucleotide bases (or other genotypic feature) of the two or more nucleic acid sequences. The term “homologous nucleic acids” generally refers to nucleic acids comprising nucleotide sequences having a degree of similarity in nucleotide base composition, arrangement, or order. The two or more nucleic acids may be of the same or different species or group. The term “percent homology” when used in relation to nucleic acid sequences, refers generally to a percent degree of similarity between the nucleotide sequences of two or more nucleic acids.

When used in relation to polypeptide (or protein) sequences, the term “homology” refers to the degree of similarity between two or more polypeptide (or protein) sequences (e.g., genes) or fragments thereof. Typically, the degree of similarity between two or more polypeptide (or protein) sequences refers to the degree of similarity of the composition, order, or arrangement of two or more amino acid of the two or more polypeptides (or proteins). The two or more polypeptides (or proteins) may be of the same or different species or group. The term “percent homology” when used in relation to polypeptide (or protein) sequences, refers generally to a percent degree of similarity between the amino acid sequences of two or more polypeptide (or protein) sequences. The term “homologous polypeptides” or “homologous proteins” generally refers to polypeptides or proteins, respectively, that have amino acid sequences and functions that are similar. Such homologous polypeptides or proteins may be related by having amino acid sequences and functions that are similar, but are derived or evolved from different or the same species using the techniques described herein.

The term “subject” as used herein includes, but is not limited to, an organism; a mammal, including, e.g., a human, non-human primate (e.g., baboon, orangutan, monkey), mouse, pig, cow, goat, cat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate.

A nucleic acid is “operably linked” with another nucleic acid sequence when it is placed into a functional relationship with another nucleic acid sequence. For instance, a promoter or enhancer is operably linked to a coding sequence if it increases the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

The term “pharmaceutical composition” means a composition suitable for pharmaceutical use in a subject, including an animal or human. A pharmaceutical composition generally comprises an effective amount of an active agent and a carrier, including, e.g., a pharmaceutically acceptable carrier.

The term “effective amount” means a dosage or amount sufficient to produce a desired result. The desired result may comprise an objective or subjective improvement in the recipient of the dosage or amount.

A “prophylactic treatment” is a treatment administered to a subject who does not display signs or symptoms of a disease, pathology, or medical disorder, or displays only early signs or symptoms of a disease, pathology, or disorder, such that treatment is administered for the purpose of diminishing, preventing, or decreasing the risk of developing the disease, pathology, or medical disorder. A prophylactic treatment functions as a preventative treatment against a disease or disorder. A “prophylactic activity” is an activity of an agent, such as a nucleic acid, vector, gene, polypeptide, protein, substance, or composition thereof that, when administered to a subject who does not display signs or symptoms of pathology, disease or disorder, or who displays only early signs or symptoms of pathology, disease, or disorder, diminishes, prevents, or decreases the risk of the subject developing a pathology, disease, or disorder. A “prophylactically useful” agent or compound (e.g., nucleic acid or polypeptide) refers to an agent or compound that is useful in diminishing, preventing, treating, or decreasing development of pathology, disease or disorder.

A “therapeutic treatment” is a treatment administered to a subject who displays symptoms or signs of pathology, disease, or disorder, in which treatment is administered to the subject for the purpose of diminishing or eliminating those signs or symptoms of pathology, disease, or disorder. A “therapeutic activity” is an activity of an agent, such as a nucleic acid, vector, gene, polypeptide, protein, substance, or composition thereof, that eliminates or diminishes signs or symptoms of pathology, disease or disorder, when administered to a subject suffering from such signs or symptoms. A “therapeutically useful” agent or compound (e.g., nucleic acid or polypeptide) indicates that an agent or compound is useful in diminishing, treating, or eliminating such signs or symptoms of a pathology, disease or disorder.

The term “gene” broadly refers to any segment of DNA associated with a biological function. Genes include coding sequences and/or regulatory sequences required for their expression. Genes also include non-expressed DNA nucleic acid segments that, e.g., form recognition sequences for other proteins (e.g., promoter, enhancer, or other regulatory regions). Genes can be obtained from a variety of sources, including cloning from a source of interest or synthesizing from known or predicted sequence information, and may include sequences designed to have desired parameters.

As used herein, an “antibody” refers to a peptide, polypeptide, or protein comprising one or more peptides or polypeptides substantially or partially encoded by at least one immunoglobulin nucleic acid molecule or immunoglobulin gene or fragment of at least one immunoglobulin molecule or immunoglobulin gene. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. Antibodies exist as intact immunoglobulins or as a number of well characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into an Fab′ monomer. The Fab′ monomer is essentially an Fab with part of the hinge region (see Fundamental Immunology, W. E. Paul, ed., Raven Press, N.Y. (1993), for a more detailed description of other antibody fragments). While various antibody fragments are defined in terms of the digestion of an intact antibody, one of ordinary skill in the art will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Antibodies include single chain antibodies, including single chain Fv (sFv) antibodies in which a variable heavy and a variable light chain are joined together (directly or through a peptide linker) to form a continuous polypeptide.

An “antigen-binding fragment” of an antibody is a peptide or polypeptide fragment of the antibody that binds an antigen. An antigen-binding site is formed by those amino acids of the antibody that contribute to, are involved in, or affect the binding of the antigen. See Scott, T. A. and Mercer, E. I., CONCISE ENCYCLOPEDIA: BIOCHEMISTRY AND MOLECULAR BIOLOGY (de Gruyter, 3d ed. 1997) [hereinafter “Scott, CONCISE ENCYCLOPEDIA”] and Watson, J. D. et al., RECOMBINAN DNA (2d ed. 1992) [hereinafter “Watson, RECOMBIlNANT DNA”], each of which is incorporated herein by reference in its entirety for all purposes.

A “stress resistant retrovirus envelope protein” or a “shear resistant retrovirus envelope protein” is a protein (polypeptide or fragment thereof) that is capable of withstanding ultracentrifugation at a force of at least about 90,000×g, 100,000×g: 110,000 x g, 120,000×g for a period of at least about 40, 50, 55, 60, 65, 68, 70, 75, 80, 85, or 90 minutes or more, particularly when incorporated into a retrovirus as an envelope protein. Under such ultracentrifugation conditions, a retrovirus comprising the stress or shear resistant retrovirus envelope protein as an envelope protein exhibits or maintains a percentage of infectious titer level relative that is at least about 50%, 60%, 70%, 80%, 90%, 91%, 92%, 93%, 94%, 95%, 96%, 97%, 98%, 99%, or 100% of the infectious titer level of an identical retrovirus comprising the stress or shear resistant retrovirus envelope protein as an envelope protein, respectively.

“Infectivity” or “infectious titer” refers to the number of viral particles that can infect a target cell (as detected by standard virological techniques commonly known to those of ordinary skill in the art) per milliliter of culture medium or viral solution or viral stock. Such standard virological techniques include, e.g., marker rescue assay and limiting-dilution vector rescue assay (described in detail below), plaque assay, and the like, such as described in, e.g., RETROVIRUSES, Coffin, John M. et al., eds. Cold Spring Harbor Labor. Press, (1997), which is incorporated herein by reference in its entirety for all purposes.

As used herein, “stress resistant” refers to a physical property of a body (e.g., molecule) in which the body withstands, remains firm against the action or effect of, or resists an applied force (e.g., stress-producing force) or system of forces that tends to alter, strain, deform, or damage the body. An example of such an applied force or system of forces is the force or system of forces experienced by a body (e.g., molecule or population of molecules) during ultracentrifugation of said body, such as, e.g., under ultracentrifugation conditions at a force(s) of least about 90,000×g, 100,000×g, 1 10,000×g, 120,000×g, or 130,000×g for a period of at least about 40, 50, 55, 60, 65, 68, 70, 75, 80, 85, or 90 minutes or more.

As used herein, a “shearing strain” is a condition in or deformation of an elastic body (e.g., molecule) caused by at least one applied force that tends to produce an opposite but parallel sliding motion of the body's planes. An example of such at least one applied force is the force or system of forces experienced by a body (e.g., molecule or population of molecules) during ultracentrifugation of said body, such as, e.g., under ultracentrifugation conditions at a force(s) of least about 90,000×g, 100,000×g, 110,000×g, 120,000×g, or 130,000×g for a period of at least about 40, 50, 55, 60, 65, 68, 70, 75, 80, 85, or 90 minutes or more.

As used herein, “shear resistant” refers to a physical property of a body (e.g., molecule) in which the body withstands, remains firm against the action or effect of or resists an applied force (e.g., shear-producing force or “shearing force”) or system of forces that tends to produce a shearing strain (e.g., ultracentrifugation of said body at a force(s) of least about 90,000×g, 100,000×g, 110,000×g, 120,000×g, or 130,000×g for a period of at least about 40, 50, 55, 60, 65, 68, 70, 75, 80, 85, or 90 minutes or more).

Generally, the nomenclature used hereafter and the laboratory procedures in cell culture, molecular genetics, molecular biology, nucleic acid chemistry, and protein chemistry described below are those well known and commonly employed by those of ordinary skill in the art. Standard techniques, such as described in Sambrook et al., Molecular Cloning—A Laboratory Manual (2nd Ed.), Vols. 1-3, Cold Spring Harbor Laboratory, Cold Spring Harbor, N.Y., 1989 (hereinafter “Sambrook”) and Current Protocols in Molecular Biology, F. M. Ausubel et al., eds., Current Protocols, a joint venture between Greene Publishing Associates, Inc. and John Wiley & Sons, Inc. (1994, supplemented through 1999) (hereinafter “Ausubel”), are used for recombinant nucleic acid methods, nucleic acid synthesis, cell culture methods, and transgene incorporation, e.g., electroporation, injection, gene gun, impressing through the skin, and lipofection. Generally, oligonucleotide synthesis and purification steps are performed according to specifications. The techniques and procedures are generally performed according to conventional methods in the art and various general references which are provided throughout this document. The procedures therein are believed to be well known to those of ordinary skill in the art and are provided for the convenience of the reader.

A variety of additional terms are defined or otherwise characterized herein.

POLYNUCLEOTIDES OF THE INVENTION

The invention provides isolated, modified or recombinant stress and/or shear retrovirus envelope protein polypeptides, and isolated, modified or recombinant stress and/or shear resistant polynucleotides encoding such polypeptides. As described in more detail below, in accordance with the present invention, polynucleotide sequences that encode novel retrovirus envelope proteins, fragments thereof, related fusion polypeptides or proteins, or functional equivalents thereof, are collectively referred to herein as “retrovirus envelope protein nucleic acids,” “stress resistant retrovirus envelope protein nucleic acids,” “shear resistant retrovirus envelope protein nucleic acids,” or “recombinant retrovirus envelope protein nucleic acids,” or, simply, “envelope protein nucleic acids or “envelope nucleic acids.”

Similarly, the retrovirus envelope proteins of the invention, fragments thereof, related fusion proteins, or functional equivalents thereof, are collectively referred to herein as “stress resistant retrovirus envelope proteins” or “shear resistant retrovirus envelope proteins”). Stress resistant retrovirus envelope protein polypeptides and shear resistant retrovirus envelope protein polypeptides are also included.

A retrovirus envelope protein nucleic acid of the invention comprises a nucleic acid of the invention that encodes a stress (and/or shear) resistant retrovirus envelope protein or a fragment thereof. A stress and/or shear resistant retrovirus of the invention comprises a polypeptide of the invention (e.g., stress and/or shear resistant retrovirus envelope protein (or fragment thereof) that confers stress and/or shear resistance on the retrovirus. Such polypeptide is incorporated into the retrovirus is an envelope protein. A stress or shear resistant retrovirus comprises a polypeptide encoded by a nucleic acid of the invention.

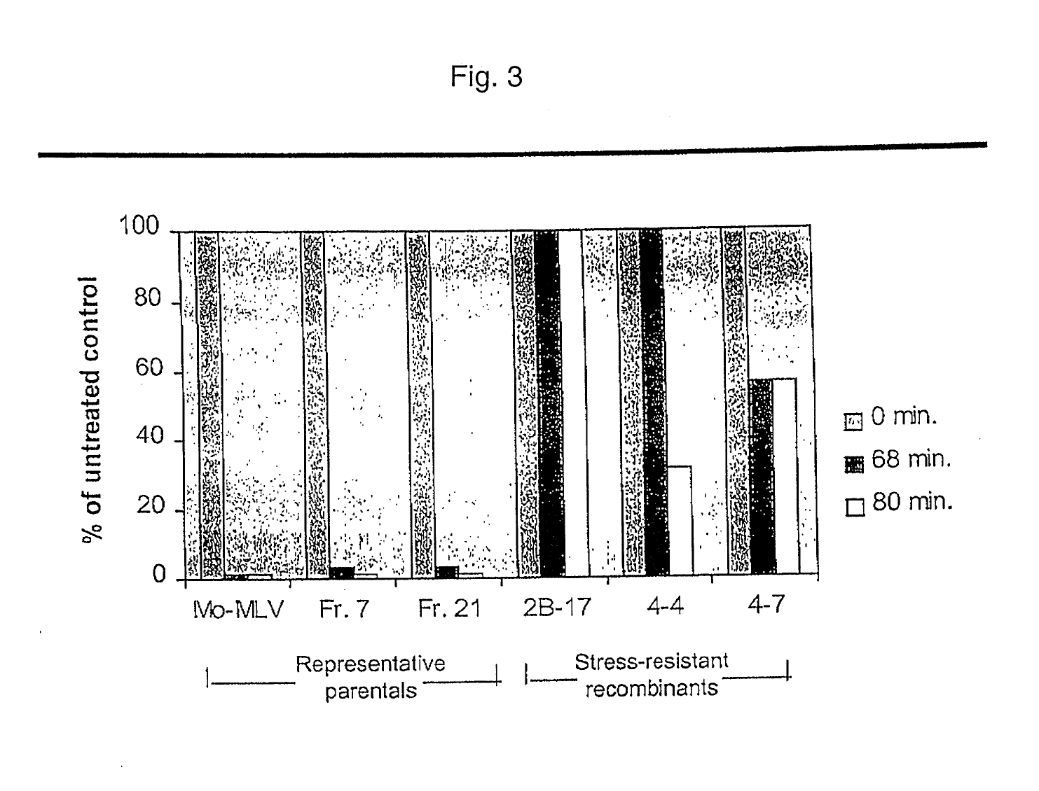

With a nucleic acid encoding a stress (and/or shear) resistant retrovirus envelope protein of the invention incorporated into a retrovirus as an envelope protein, the retrovirus can withstand ultracentrifugation at a force of at least about 90,000×g, 100,000×g, 110,000×g, or 120,000×g for at least about 20, 30, 40, 50, 55, 60, 65, 68, 70, 75, 80, 85, or 90 minutes or more while maintaining an infectious titer level of at least about 10%, 20%, 25%, 30%, 40%, 50%, 60%, 70%, 80%, 90%, 91%, 92%, 93%, 94%, 95%, 96%, 97%, 98%, 99%, or 100% of the infectious titer level of an identical retrovirus incorporating said stress and/or shear resistant envelope protein polypeptide that has not undergone ultracentrifugation under the same conditions. See, e.g., FIG. 3. Other ultracentrifugation conditions that produce a substantially identical or equivalent stress or shear force or shearing strain can also be employed for identifying stress or shear resistant envelope protein when such protein is incorporated as an envelope protein into a retrovirus that is subjected to such ultracentrifugation conditions.

Preferentially, in some embodiments, the stress and/or shear resistant polypeptide (or fragment thereof) encoded by the nucleic acid of the invention, when incorporated into a retrovirus as an envelope protein, confers upon the retrovirus the ability to withstanding ultracentrifugation of a force in excess of at least about 120,000×g for at least about 50, 60, 55, 65, 68, 70, 75, 80, 85, or 90 minutes or more, such that it retains or preserves an infectious titer level of at least about 50%, 60%, 70%, 80%, 85%, 90%, 91%, 92%, 93%, 94%, 95%, 96%, 97%, 98%, 99% or 100% relative to the infectious titer level of an identical retrovirus comprising said stress and/or shear resistant retrovirus envelope protein polypeptide (or fragment thereof) that has not been subjected to such ultracentrifugation conditions.

Retroviruses and retroviral particles, and components thereof, including the viral envelope comprising a lipid bilayer into which proteins encoded by the env region of the viral genome are inserted (e.g., envelope proteins), are described in detail in RETROVIRUSES (Coffin, John M. et al., eds. Cold Spring Harbor Laboratory Press (1997), supra (see, e.g., Chap. 1). Polynucleotides encoding the polypeptides of the invention were selected for stress (and/or shear) resistance by amplification of retroviruses comprising the envelope protein polypeptides following concentration by ultracentrifugation for various time periods as described in greater detail in the Examples below. Coding sequences for the novel envelope protein polypeptides were identified using well known genetic algorithms (GAs) as described herein.

Briefly, recombinant envelope (env) genes derived from murine ecotropic retroviruses were ligated into a replication competent retrovirus (RCR) backbone (e.g., Moloney RCR backbone) and the RCR vector comprising the recombinant env sequences was transfected into 293T cells to generate a set of recombinant retroviruses. See, e.g., analogous methods described in Soong, N. W. et al. (2000) Nature Genetics 25:436-439 (2000).

Stress (and/or shear) resistant viruses can be selected, for example, by repeated ultracentrifugation under defined conditions (e.g., 120,000×g for 68 minutes) with amplification of surviving viruses. After three rounds of selection and amplification, the resulting stress (and/or shear) resistant viruses were cloned and analyzed. Exemplary nucleic acids of the invention that encode stress (and/or shear) resistant retrovirus envelope protein polypeptides are identified herein as SEQ ID NO:1 to SEQ ID NO:3, and complementary sequences or fragments thereof, which encode retrovirus envelope protein polypeptides identified herein as SEQ ID NO:4 to SEQ ID NO:6, respectively.

Also contemplated by the present invention are those nucleic acid sequences which, due to the inherent degeneracy of the genetic code, encode substantially the same or a functionally equivalent stress and/or shear resistant retrovirus envelope protein polypeptides of the invention. The present invention includes, for example, degenerate nucleotide sequences of SEQ ID NOS:1-3 (or fragments thereof) that encode one or more retrovirus envelope protein polypeptides identified herein as SEQ ID NOS:4-6 (or fragments thereof), respectively, or functionally equivalent stress and/or shear resistant retrovirus envelope protein polypeptides. The invention also includes degenerate nucleotide sequences of SEQ ID NOS:9-10 (or fragments thereof) that encode one or more retrovirus envelope protein polypeptides identified herein as SEQ ID NOS:7-8 (or fragments thereof), respectively, or functionally equivalent retrovirus envelope protein polypeptides.

Additionally, the present invention includes an isolated or recombinant nucleic acid comprising a nucleic acid sequence that encodes a stress and/or shear resistant retrovirus envelope protein polypeptide having an amino acid sequence of any one of SEQ ID NOS:4-6 that comprises or contains one or more conservatively modified variations, e.g., one or more substitutions, additions, or deletions that alter, add, or delete a single amino acid or a small percentage of amino acids (typically less than about 5%, more typically less than about 4%, 2%, or 1%), as described in greater detail below.

Also included are isolated or recombinant nucleic acids comprising a nucleic acid sequence that encodes retrovirus envelope protein polypeptide having an amino acid sequence of any one of SEQ ID NOS:7-8 that comprises or contains one or more conservatively modified variations, e.g., one or more substitutions, additions, or deletions that alter, add, or delete a single amino acid or a small percentage of amino acids (typically less than about 5%, more typically less than about 4%, 2%, or 1%).

In another aspect, the invention provides an isolated or recombinant nucleic acid, comprising a polynucleotide sequence selected from the group consisting of: (a) SEQ ID NOS:9-10, or a complementary polynucleotide sequence thereof; (b) a polynucleotide sequence encoding a polypeptide selected from SEQ ID NOS:7-8, or a complementary polynucleotide sequence thereof; (c) a polynucleotide sequence that hybridizes under highly stringent conditions over substantially the entire length of polynucleotide sequence (a) or (b); and (d) a polynucleotide sequence comprising a fragment of (a), (b), or (c), said fragment encoding a stress or shear resistant retrovirus polypeptide capable of withstanding centrifugation or ultracentrifugation at a force of at least about 120,000×g for at least about 30 minutes.

As described in greater detail below, the polynucleotides of the invention are useful in for a variety of applications, including, but not limited to, in recombinant production of the recombinant envelope protein polypeptides of the invention; as therapeutics or prophylactics, for use in, e.g., gene therapy and related methodologies; as immunogens; and as diagnostic probes, e.g., for the presence of complementary or partially complementary nucleic acids (including for detection of natural retroviral coding nucleic acids).

In another aspect, the invention provides an isolated or recombinant nucleic acid that comprises a polynucleotide sequence selected from the group consisting of: (a) SEQ ID NOS:1-3 and/or SEQ ID NOS:9-10, or a complementary polynucleotide sequence thereof; (b) a polynucleotide sequence encoding a polypeptide selected from SEQ ID NOS:.4-8, or a complementary polynucleotide sequence thereof; (c) a polynucleotide sequence that hybridizes under at least highly stringent conditions, ultra-high stringency conditions, ultra-ultra-high stringency conditions, or very stringent conditions (as defined or illustrated herein) over substantially the entire length of polynucleotide sequence (a) or (b); (d) a polynucleotide sequence comprising a fragment of (a), (b), or (c), which fragment encodes all or a part of a stress and/or shear resistant retrovirus envelope protein; (e) polynucleotide sequence which, but for the degeneracy of the genetic code, hybridizes under at least highly stringent conditions, ultra-high stringency conditions, ultra-ultra-high stringency conditions, or very stringent conditions over substantially the entire length of polynucleotide sequence (a), (b), (c), or (d); and (f) a polynucleotide sequence comprising a fragment of a polynucleotide sequence of (e), which fragment encodes all or part of a stress or shear resistant retrovirus envelope protein.

Examples of isolated or recombinant DNAs of the invention include a polynucleotide sequence selected from the group consisting of: (a) SEQ ID NOS:1-3 and/or SEQ ID NOS:9-10, or a complementary polynucleotide sequence thereof; (b) a polynucleotide sequence encoding a polypeptide selected from SEQ ID NOS:4-8 (or an amino acid sequence substantially the same as that shown in SEQ ID NOS:4-8, or a complementary polynucleotide sequence thereof; (c) a polynucleotide sequence which hybridizes under at least moderately stringent, highly stringent conditions, or very stringent conditions over substantially the entire length of polynucleotide sequence (a) or (b), or with a nucleotide fragment or nucleotide portion comprising at least about 50, 100, 200, 300, 400, 500, 550, 600, 650, 1000, 1500, 1750, 1800, 1900, 2000, or 2025 nucleotides of a sequence defined in (a) or (b); and (d) a polynucleotide sequence comprising a fragment of (a), (b), or (c), which fragment encodes all or a part of a retrovirus envelope protein. Some such fragments exhibit stress-resistant properties such that they are able to withstand stress forces during purification and centrifugation.

Nucleic Acid Hybridization

Nucleic acids “hybridize” when they associate, typically in solution. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, New York), as well as in Ausubel, supra. Hames and Higgins (1995) Gene Probes 1, IRL Press at Oxford University Press, Oxford, England, (“Hames and Higgins 1”) and Hames and Higgins (1995) Gene Probes 2, IRL Press at Oxford University Press, Oxford, England (“Hames and Higgins 2”) provide details on the synthesis, labeling, detection and quantification of DNA and RNA, including oligonucleotides.

An indication that two nucleic acid sequences are substantially identical is that the two molecules hybridize to each other under at least stringent conditions. The phrase “hybridizing specifically to” (or “hybridizes specifically to”) refers to the binding, duplexing, or hybridizing of a molecule only to a particular nucleotide sequence under stringent conditions when that sequence is present in a complex mixture (e.g., total cellular DNA or RNA). “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

“Stringent hybridization wash conditions” in the context of nucleic acid hybridization experiments such as Southern and northern hybridizations are sequence dependent and are different under different environmental parameters. An extensive guide to the hybridization of nucleic acids is found in Tijssen (1993), supra, and in Hames and Higgins 1 and Hames and Higgins 2, supra.

For purposes of the present invention, generally, “highly stringent” hybridization and wash conditions are selected to be about 50C lower than the thermal melting point (Tₘ) for the specific sequence at a defined ionic strength and pH (as noted above, highly stringent conditions can also be referred to in comparative terms). The Tₘ is the temperature (under defined ionic strength and pH) at which 50% of the test sequence hybridizes to a perfectly matched probe. Very stringent conditions are selected to be equal to the Tₘ for a particular probe. The Tₘ is the temperature of the nucleic acid duplexes indicates the temperature at which the duplex is 50% denatured under the given conditions and its represents a direct measure of the stability of the nucleic acid hybrid. Thus, the Tₘ corresponds to the temperature corresponding to the midpoint in transition from helix to random coil; it depends on length, nucleotide composition, and ionic strength for long stretches of nucleotides.

After hybridization, unhybridized nucleic acid material can be removed by a series of washes, the stringency of which can be adjusted depending upon the desired results. Low stringency washing conditions (e.g., using higher salt and lower temperature) increase sensitivity, but can product nonspecific hybridization signals and high background signals. Higher stringency conditions (e.g., using lower salt and higher temperature that is closer to the hybridization temperature) lowers the background signal, typically with only the specific signal remaining. See Rapley, R. and Walker, J. M. eds., Molecular Biomethods Handbook (Humana Press, Inc. 1998) (hereinafter “Rapley and Walker”), which is incorporated herein in its entirety for all purposes.

The Tₘ of a DNA-DNA duplex can be estimated using the following equation (1):

Tₘ(° C.)=81.5° C.+16.6(log₁₀ M)+0.41(%G+C)−0.72(%f)−500/n,

where M is the molarity of the monovalent cations (usually Na+), (%G+C) is the percentage of guanosine (G) and cystosine (C ) nucleotides, (%f) is the percentage of formalize and n is the number of nucleotide bases (i.e., length) of the hybrid. Id.

The Tₘ of an RNA-DNA duplex can be estimated by equation (2) as follows:

Tₘ(° C.)=79.8° C.+18.5(log₁₀ M)+0.58(%G+C)−11.8(%G+C)²−0.56(%f)−820/n,

where M is the molarity of the monovalent cations (usually Na+), (%G+C) is the percentage of guanosine (G ) and cystosine (C ) nucleotides, (%f) is the percentage of formamide and n is the number of nucleotide bases (i.e., length) of the hybrid. See Rapley and Walker, supra.

Equations 1 and 2 typically accurate only for hybrid duplexes longer than about 100-200 nucleotides. Id.

The Tₘ of nucleic acid sequences shorter than 50 nucleotides can be calculated as follows:

Tₘ(° C.)=4(G+C)+2(A+T),

where A (adenine), C, T (thymine), and G are the numbers of the corresponding nucleotides.

As noted “highly stringent” conditions are selected to be about 5° C. or less lower than the thermal melting point (Tₘ) for the specific sequence at a defined ionic strength and pH. Target sequences that are closely related or identical to the nucleotide sequence of interest (e.g., “probe”) can be identified under highly stringent conditions. Lower stringency conditions are appropriate for sequences that are less complementary. See, e.g., Rapley and Walker, supra.

An example of stringent hybridization conditions for hybridization of complementary nucleic acids that have more than 100 complementary residues on a filter in a Southern or northern blot is 50% formalin with 1 mg of heparin at 42° C., with the hybridization being carried out overnight. An example of stringent wash conditions is a 0.2× SSC wash at 65° C. for 15 minutes (see Sambrook, supra, for a description of SSC buffer). Often the high stringency wash is preceded by a low stringency wash to remove background probe signal. An example low stringency wash is 2x SSC at 40° C. for 15 minutes.

Comparative hybridization can be used to identify nucleic acids of the invention, and this comparative hybridization method is a preferred method of distinguishing nucleic acids of the invention.

Detection of stringent hybridization in the context of the present invention indicates relatively strong structural similarity/homology to, e.g., the nucleic acids provided in the sequence listings herein. Highly stringent hybridization between two nucleotide sequences demonstrates a degree of similarity or homology of structure, nucleotide base composition, arrangement or order that is greater than that detected by stringent hybridization conditions. In particular, detection of highly stringent hybridization in the context of the present invention indicates strong structural similarity or structural homology (e.g., nucleotide structure, base composition, arrangement or order) to, e.g., the nucleic acids provided in the sequence listings herein. For example, it is desirable to identify test nucleic acids that hybridize to the exemplar nucleic acids herein under stringent conditions. Thus, one measure of stringent hybridization is the ability to hybridize to one of the listed nucleic acids (e.g., nucleic acid sequences SEQ ID NOS:1-3 and/or SEQ ID NOS:9-10, and complementary polynucleotide sequences thereof) under highly stringent conditions or very stringent conditions (or ultra-high stringency hybridization conditions, or ultra-ultra high stringency hybridization conditions). Stringent or highly stringent hybridization conditions and wash conditions (or ultra-high stringency, or ultra-ultra high stringency conditions) can easily be determined empirically for any test nucleic acid.

For example, in determining highly stringent hybridization and wash conditions, the hybridization and wash conditions are gradually increased (e.g., by increasing temperature, decreasing salt concentration, increasing detergent concentration and/or increasing the concentration of organic solvents, such as formalin, in the hybridization or wash), until a selected set of criteria are met. For example, the hybridization and wash conditions are gradually increased until a probe comprising one or more nucleic acid sequences selected from the group consisting of SEQ ID NOS:1-3 and/or SEQ ID NOS:9-10 and complementary polynucleotide sequences thereof, binds to a perfectly matched complementary target (again, a nucleic acid comprising one or more nucleic acid sequences selected from SEQ ID NOS:1-3 and/or SEQ ID NOS:9-10 and complementary polynucleotide sequences thereof), with a signal to noise ratio that is at least 2.5× and optionally 5× or more as high as that observed for hybridization of the probe to an unmatched target. In this case, the unmatched target is a nucleic acid corresponding to a known MLV envelope protein, e.g., an MLV envelope protein nucleic acid that is present in a public database such as GenBankTm at the time of filing of the subject application. Examples of such unmatched target nucleic acids include, e.g., those with the following GenBank accession numbers: S80869, S77017, S77015, S77012, J01998, AF169256, Z35109, K02714, Y13893, D88386, Z11128, M93134, X02794, AF288940, U94692, AF030173, and AH000833, or other GenBank accession numbers discussed herein, or other similar MLV sequences presented in GenBank. Additional such sequences can be identified in GenBank by one of ordinary skill in the art.

A test nucleic acid is said to specifically hybridize to a probe nucleic acid when it hybridizes at least ½ as well to the probe as to the perfectly matched complementary target, i.e., with a signal to noise ratio at least ½ as high as hybridization of the probe to the target under conditions in which the perfectly matched probe binds to the perfectly matched complementary target with a signal to noise ratio that is at least about 2.5×-10×, typically, 5×-10× as high as that observed for hybridization to any of the unmatched target nucleic acids represented by GenBank accession numbers S80869, S77017, S77015, S77012, J01998, AF169256, Z35109, K02714, Y13893, D88386, Z11128, M93134, X02794, AF288940, U94692, AF030173, and AH000833, or other GenBank accession numbers discussed herein, or other similar MLV sequences presented in GenBank.

In one aspect, the invention provides a target nucleic acid that hybridizes under stringent conditions to a unique coding oligonucleotide that encodes a unique subsequence in a polypeptide selected from any of SEQ ID NOS:4-8, where the unique subsequence is unique compared to a polypeptide encoded by any of above GenBank Nucleotide Accession Numbers or known retrovirus envelope protein. For some such nucleic acids, the stringent conditions are selected such that a perfectly complementary oligonucleotide to the coding oligonucleotide hybridizes to the coding oligonucleotide with at least about a 5× higher signal to noise ratio than for hybridization of the perfectly complementary oligonucleotide to a control nucleic acid corresponding to any of GenBank Nucleotide Accession Nos. set forth above and below.

Ultra high-stringency hybridization and wash conditions are those in which the stringency of hybridization and wash conditions are increased until the signal to noise ratio for binding of the probe to the perfectly matched complementary target nucleic acid is at least 10× as high as that observed for hybridization to any of the unmatched target nucleic acids represented by GenBank accession numbers S80869, S77017, S77015, S77012, J01998, AF169256, Z35109, K02714, Y13893, D88386, Z11128, M93134, X02794, AF288940, U94692, AF030173, and AH000833, or other GenBank accession numbers discussed herein, or other similar MLV sequences presented in GenBank. A target nucleic acid that hybridizes to a probe under such conditions, with a signal to noise ratio of at least /2 that of the perfectly matched complementary target nucleic acid, is said to bind to the probe under ultra-high stringency conditions.

Similarly, even higher levels of stringency can be determined by gradually increasing the hybridization and/or wash conditions of the relevant hybridization assay. For example, those in which the stringency of hybridization and wash conditions are increased until the signal to noise ratio for binding of the probe to the perfectly matched complementary target nucleic acid is at least 10×, 20×, 50×, 100×, or 500× or more as high as that observed for hybridization to any of the unmatched target nucleic acids represented by GenBank accession numbers S80869, S77017, S77015, S77012, J01998, AF169256, Z35109, K02714, Y13893, D88386, Z11128, M93134, X02794, AF288940, U94692, AF030173, and AH000833, or other GenBank accession numbers discussed herein, or other similar MLV sequences presented in GenBank, can be identified. A target nucleic acid that hybridizes to a probe under such conditions, with a signal to noise ratio of at least ½ that of the perfectly matched complementary target nucleic acid, is said to bind to the probe under ultra-ultra-high stringency conditions.

Target nucleic acids that hybridize to the nucleic acids represented by SEQ ID NOS:1-3 and/or SEQ ID NOS:9-10 under stringent conditions and high, ultra-high, and ultra-ultra high stringency conditions are a feature of the invention. Examples of such nucleic acids include those with one or a few silent or conservative nucleic acid substitutions as compared to a given nucleic acid sequence.

Nucleic acids that do not hybridize to each other under stringent conditions are still substantially identical if the polypeptides that they encode are substantially identical. This occurs, e.g., when a copy of a nucleic acid is created using the maximum codon degeneracy permitted by the genetic code, including, e.g., the following MLV sequences in GenBank: GenBank accession numbers AAB34096; AAB34095; and AAB34094, or other similar MLV sequences presented in GenBank.

Additionally, for distinguishing between duplexes with sequences of less than about 100 nucleotides, a TMACI hybridization procedure known to those of ordinary skill in the art can be used. See, e.g., Sorg, U. et al., 1 Nucleic Acids Res (Sep. 11, 1991)19(17), incorporated herein by reference in its entirety for all purposes.

In another aspect, the invention provides a nucleic acid that comprises a unique subsequence in a nucleic acid selected from SEQ ID NOS:1-3 and/or SEQ ID NOS:9-10. The unique subsequence is unique as compared to a naturally occurring or known nucleic acid retrovirus sequence, such as a naturally occurring or known MLV nucleic acid sequence. Representative known sequences include, for example those represented by GenBank accession numbers S80869, S77017, S77015, S77012, J01998, AF169256, Z35109, K02714, Y13893, D88386, Z11128, M93134, X02794, AF288940, U94692, AF030173, and AH000833, or other GenBank accession numbers discussed herein, or other similar MLV sequences presented in GenBank. Such unique subsequences can be determined by aligning any of SEQ ID NOS:1-3 and/or SEQ ID NOS:9-10 against the complete set of nucleic acids corresponding to GenBank accession numbers S80869, S77017, S77015, S77012, J01998, AF169256, Z35109, K02714, Y13893, D88386, Z11128, M93134, X02794, AF288940, U94692, AF030173, AH000833, or other GenBank accession numbers discussed herein, or other similar MLV sequences presented in GenBank, and other naturally occurring or known retrovirus sequences. Alignment can be performed using the BLAST algorithm set to default parameters. Any unique subsequence is useful, e.g., as a probe to identify the nucleic acids of the invention.

The present invention also includes a polypeptide that comprises a unique subsequence in a polypeptide selected from any of SEQ ID NOS:4-8, wherein the unique subsequence is unique as compared to an amino acid sequence, polypeptide sequence or polypeptide subsequence of a naturally occurring or known retroviral envelope protein, such as a naturally occurring or known MLV envelope protein sequence. Representative known amino acid sequences include those corresponding to any of GenBank accession numbers AAB34096, AAB34095, and AAB34094, or other naturally occurring or known retrovirus envelope protein sequences, including, e.g., an MLV envelope protein polypeptide. Here again, the polypeptide is aligned against the complete set of polypeptides corresponding to GenBank accession numbers AAB34096, AAB34095, AAB34094, and/or other naturally occurring or known retrovirus sequences (the control polypeptides). Note that where a control sequence corresponds to a non-translated sequence such as a pseudo gene, the corresponding polypeptide is generated simply by translation of the nucleic acid sequence into an amino acid sequence (since each nucleotide triplet codon encodes one or more known amino acids), where the reading frame is selected to correspond to the reading frame of a homologous envelope protein nucleic acids; how to perform such translation would be readily known to those of ordinary skill in the art.

In addition, the present invention provides a target nucleic acid that hybridizes under at least stringent or highly stringent conditions (or conditions of greater stringency) to a unique coding oligonucleotide that encodes a unique amino acid subsequence in a polypeptide selected from SEQ ID NOS:4-8, wherein the unique amino acid subsequence is unique as compared to an amino acid subsequence of a known or naturally occurring retrovirus envelope protein polypeptide, including, e.g., an MLV envelope protein polypeptide. Unique sequences are determined as noted above.

The invention also provides for a target nucleic acid that hybridizes under at least stringent conditions, highly stringent, ultra-high stringency conditions, or ultra-ultra-high stringency conditions to a unique coding oligonucleotide that encodes a unique subsequence in a polypeptide selected from: SEQ ID NOS:4-8, wherein the unique subsequence is unique as compared to a polypeptide corresponding to any of the control polypeptides. Unique sequences are determined as noted above.

In one example, the stringent conditions are selected such that a perfectly complementary oligonucleotide to the coding oligonucleotide hybridizes to the coding oligonucleotide with at least about a 5-10× higher signal to noise ratio than for hybridization of the perfectly complementary oligonucleotide to a control nucleic acid corresponding to any of the control polypeptides. Conditions can be selected such that higher ratios of signal to noise are observed in the particular assay which is used, e.g., about 15×, 20×, 30×, 50×, or more. In this example, the target nucleic acid hybridizes to the unique coding oligonucleotide with at least a 2× higher signal to noise ratio as compared to hybridization of the control nucleic acid to the coding oligonucleotide. Again, higher signal to noise ratios can be selected, e.g., about 2.5×, 5×, 10×, 20×, 30×, 50× or more. The particular signal will depend on the label used in the relevant assay, e.g., a fluorescent label, a colorimetric label, a radio active label, or the like.

In another aspect, the invention provides a polypeptide that comprises a unique subsequence in a polypeptide selected from SEQ ID NOS:4-6, wherein the unique subsequence is unique as compared to a polypeptide sequence corresponding to a known MLV envelope protein, such as, e.g., an MLV envelope protein present in GenBank.

Making Polynucleotides of the Invention

Polynucleotides and oligonucleotides of the invention can be prepared by standard solid-phase methods, according to known synthetic methods. Typically, fragments of up to about 20, 30, 40, 50, 60, 70, 80, 90, and/or 100 bases are individually synthesized, then joined (e.g., by enzymatic or chemical ligation methods, or polymerase mediated recombination methods) to form essentially any desired continuous sequence. In another aspect, fragments of greater than 100 bases (e.g., 150, 200, 300, 400, 500, 600, 650, 1000, 1500 bases) are individually synthesized, then joined (e.g., by enzymatic or chemical ligation methods, or polymerase mediated recombination methods) to form essentially any desired continuous sequence.

For example, the polynucleotides and oligonucleotides of the invention, including fragments thereof (and those as described above), can be prepared by chemical synthesis using, e.g., the classical phosphoramidite method described by Beaucage et al. (1981) Tetrahedron Letters 22:1859-69, or the method described by Matthes et al. (1984) EMBO J. 3:801-05, e.g., as is typically practiced in automated synthetic methods. According to the phosphoramidite method, oligonucleotides are synthesized, e.g., in an automatic DNA synthesizer, purified, annealed, ligated and cloned in appropriate vectors.

In addition, essentially any nucleic acid can be custom ordered from any of a variety of commercial sources, such as The Midland Certified Reagent Company (mcrc@oligos.com), The Great American Gene Company (http://www.genco.com), ExpressGen Inc. (www.expressgen.com), Operon Technologies Inc. (Alameda, Calif.) and many others. Similarly, peptides and antibodies can be custom ordered from any of a variety of sources, such as PeptidoGenic (pkim@ccnet.com), HTI Bio-Products, Inc. (http:Hlwww.htibio.com), BMA Biomedicals Ltd (U.K.), Bio. Synthesis, Inc., and many others.

Certain polynucleotides of the invention may also be obtained by screening cDNA libraries using oligonucleotide probes that can hybridize to or PCR-amplify polynucleotides that encode the envelope protein polypeptides and fragments of those polypeptides. Procedures for screening and isolating cDNA clones are well-known to those of ordinary skill in the art. Such techniques are described in, for example, Sambrook et al. (1989) supra, and Ausubel, FM et al. (1989; supplemented through 1999), supra.

As described in more detail herein, the polynucleotides of the invention include sequences that encode novel stress resistant retrovirus envelope protein polypeptides and sequences complementary to the coding sequences, and novel fragments of coding sequences and complements thereof. The polynucleotides can be in the form of RNA or in the form of DNA, and include mRNA, cRNA, synthetic RNA and DNA, and cDNA. The polynucleotides can be double-stranded or single-stranded, and if single-stranded, can be the coding strand or the non-coding (anti-sense, complementary) strand. The polynucleotides optionally include the coding sequence of an envelope polypeptide (i) in isolation, (ii) in combination with additional coding sequence, so as to encode, e.g., a fusion protein, a pre-protein, a prepro-protein, or the like, (iii) in combination with non-coding sequences, such as introns, control elements such as a promoter, a terminator element, or 5′ and/or 3′ untranslated regions effective for expression of the coding sequence in a suitable host, and/or (iv) in a vector or host environment in which the envelope protein polypeptide coding sequence is a heterologous nucleic acid sequence or gene. Sequences can also be found in combination with typical compositional formulations of nucleic acids, including in the presence of carriers, buffers, adjuvants, excipients, and the like.

The term DNA or RNA encoding the respective stress and/or shear resistant retrovirus envelope protein polypeptide of the invention includes any oligodeoxynucleotide or oligodeoxyribonucleotide sequence which, upon expression, results in production of a retrovirus envelope protein polypeptide having the characteristics of stress and/or shear resistance. That is, the present invention includes DNA and RNA which, upon expression in an appropriate host cell, produces a retrovirus envelope protein polypeptide which, when incorporated into a retrovirus, is capable of withstanding ultracentrifugation at a force of at least about 90,000×g, 100,000×g, 110,000×g, 120,000×g for a period of at least about 40, 50, 60, 55, 65, 68, 70, 75, 80, 85, or 90 minutes or more, while retaining at least about 50%, 60%, 70%, 80%, 90%, 91%, 92%, 93%, 94%, 95%, 96%, 97%, 98%, 99%, or 100% infectious titer relative to the infectious titer of an identical retrovirus that has not undergone ultracentrifugation. The DNA or RNA can be produced in an appropriate host cell or can be produced synthetically (e.g., by an amplification technique such as PCR) or chemically.

EXPRESSION OF POLYPEPTIDES FROM POLYNUCLEOTIDES

In accordance with the present invention, polynucleotide sequences which encode novel stress and/or shear retrovirus envelope protein polypeptides, fragments of such envelope protein polypeptides, related fusion polypeptides or proteins, or functional equivalents thereof, collectively referred to herein as “retrovirus envelope protein polypeptides,” or “retrovirus envelope proteins” or, simply, “envelope protein polypeptides or “envelope proteins,” or “envelope polypeptides,” are used in recombinant DNA molecules that direct the expression of the envelope proteins or polypeptide in appropriate host cells. Due to the inherent degeneracy of the genetic code, other nucleic acid sequences which encode substantially the same or a functionally equivalent amino acid sequence are also used to clone and express the envelope proteins.

Retrovirus expression

Cells can be stably transduced with a number of viral vectors, including those derived from retroviruses (e.g., lentiviruses), including ecotropic retroviruses, pox viruses, adenoviruses (Ads), herpes viruses and parvoviruses. Common viral vectors include those derived from murine leukemia viruses (MLV), gibbon ape leukemia viruses (GaLV), human immunodeficiency viruses (HIV), adenoviruses, adeno associated viruses (AAVs), Epstein Barr viruses, canarypox viruses, cowpox viruses, and vaccinia viruses. Viral vectors based upon retroviruses, adeno-associated viruses, herpes viruses and adenoviruses are all used as gene therapy vectors for the introduction of therapeutic or prophylactic nucleic acids into the cells of an organism by ex vivo and in vivo methods.

When using viral vectors, packaging cells are commonly used to prepare virions used to transduce target cells. In these vectors, trans-active genes are rendered inactive and “rescued” by trans-complementation to provide a packaged vector. This form of trans complementation is provided by co-infection of a packaging cell with a virus or vector that supplies functions missing from a particular gene therapy vector in trans, or by using a cell line (e.g., 293T cells) having viral components integrated into the genome of the packaging cell. For instance, cells transduced with retroviral proviral sequences, e.g., MLV sequences, which lack the nucleic acid packaging site produce retroviral trans active components, but do not specifically incorporate the retroviral nucleic acids into the capsids produced, and therefore produce little or no live virus.

If these transduced “packaging” cells are subsequently transduced with a vector nucleic acid that lacks coding sequences for retroviral trans active functions, but includes a packaging signal, the vector nucleic acid is packaged into an infective virion. A number of packaging cell lines useful for MoMLV-based (Moloney MLV-based) vectors are known in the art, such as PA317 (ATCC CRL 9078), which expresses MoMLV core and envelope proteins. See Miller et al. J. Virol. 65:2220-2224 (1991). Carrol et al. (1994) J. Virol. 68(9):6047-6051 describes the construction of packaging cell lines for HIV viruses. Reciprocal complementation of defective HIV molecular clones is described, e.g., in Lori et al. (1992) J. Virol. 66(9):5553-5560. Any of these methods can be adapted to produce packaging cells expressing the envelope proteins of the invention, e.g., by incorporating a polynucleotide encoding a stress and/or shear resistant envelope protein. Viral vectors can be produced from such cells which will incorporate the shear and/or stress resistant vectors of the invention.

Functions of viral replication not supplied by trans-complementation that are necessary for replication of a vector, e.g., a vector comprising a therapeutic or prophylactic gene construct, are present in the vector.

Another common vector is based upon adenovirus. Typically, vectors which include the adenovirus ITRs (Gingeras et al. (1982) J. Biol. Chem. 257:13475-13491) are packaged in, e.g., 293T cells, which provide many of the components necessary for vector packaging. For a general review of AAVs and of the adenovirus or herpes helper functions see Bems and Bohensky (1987) Advanced in Virus Research, Academic Press, 32:243-306. The genome of AAV is described in Laughlin et al. (1983) Gene, 23:65-73. Expression of AAV is described in Beaton et al. (1989) J. Virol. 63:4450-4454. In general, the packaging sites for all parvoviruses, including B 19 and AAV are located in the viral ITRs. Recombinant AAV vectors (rAAV vectors) deliver foreign nucleic acids to a wide range of mammalian cells (Hermonat & Muzycka (1984) Proc. Nat'l Acad. Sci. USA 81:6466-6470; Tratschin et al. (1985) Mol. Cell Biol. 5:3251-3260), integrate into the host chromosome (McLaughlin et al. (1988) J. Virol. 62:1963-1973), and show stable expression of the transgene in cell and animal models (Flotte et al. (1993) Proc. Nat'l Acad. Sci. USA 90:10613-10617). rAAV vectors are able to infect non-dividing cells (Podsakoff et al. (1994) J. Virol. 68:5656-66; Flotte et al. (1994) Am. J. Respir. Cell Mol. Biol. 11:517-521).

Pseudotyping the Packageable Vector

Packageable vectors are made competent to transform target cells, e.g., CD34+ hematopoietic stem cells, by pseudotyping the vector. This is done by transducing the packaging cell line used to package the vector with a nucleic acid that encodes an Env protein which supplants or complements the retroviral env function. The envelope function can be supplied in trans by any number of heterologous viral envelope proteins. These ixiRlude, but are not limited to, VSV-G, the amphotropic envelope of Moloney murine leukemia virus (MoMuLV), and gibbon ape leukemia virus (GALV) envelope. In the context of the present invention, the packaging cell line is transduced with a nucleic acid encoding a stress or shear resistant envelope polypeptide or protein of the invention.

Modified Coding Sequences

As will be understood by those of ordinary skill in the art, it can be advantageous to modify a coding sequence (including, e.g., a nucleotide sequence encoding a stress and/or shear resistant retrovirus envelope proteins of the invention or fragments thereof) to enhance its expression in a particular host. The genetic code is redundant with 64 possible codons, but most organisms preferentially use a subset of these codons. The codons that are utilized most often in a species are called optimal codons, and those not utilized very often are classified as rare or low-usage codons (see, e.g.,, Zhang, S. P. et al. (1991) Gene 105:61-72). Codons can be substituted to reflect the preferred codon usage of the host, a process called “codon optimization” or “controlling for species codon bias.”

Optimized coding sequence containing codons preferred by a particular prokaryotic or eukaryotic host (see also Murray, E. et al. (1989) Nuc. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, preferred stop codons for S. cerevisiae and mammals are UAA and UGA respectively. The preferred stop codon for monocotyledonous plants is UGA, whereas insects and E. coli prefer to use UAA as the stop codon (Dalphin, M. E. et al. (1996) Nuc. Acids Res. 24:216-218).

The polynucleotide sequences of the present invention can be engineered in order to alter an envelope protein coding sequence for a variety of reasons, including but not limited to, alterations which modify the cloning, processing and/or expression of the gene product. For example, alterations may be introduced using techniques that are well known in the art, e.g., site-directed mutagenesis, to insert new restriction sites, to alter glycosylation patterns, to change codon preference, to introduce splice sites, etc.

Vectors, Promoters and Expression Systems

The present invention also includes isolated or recombinant constructs comprising one or more of the novel nucleic acid sequences as broadly described above (e.g., including those encoding stress and/or shear retrovirus envelope proteins of the invention or fragments thereof). The constructs comprise a vector, such as, a plasmid, a cosmid, a phage, a virus (including a retrovirus), a bacterial artificial chromosome (BAC), a yeast artificial chromosome (YAC), and the like, into which a nucleic acid sequence of the invention has been inserted, in a forward or reverse orientation. In an aspect of this embodiment, the construct further comprises regulatory sequences, including, for example, a promoter, operably linked to the sequence. Large numbers of suitable vectors and promoters are known to those of ordinary skill in the art, and are commercially available. Mutated or recombinant promoters (e.g., mutated CMV promoter), may be used with nucleic acid sequences of the invention.

General texts that describe molecular biological techniques useful herein, including the use of vectors, promoters and many other relevant topics, include Berger and Kimmel, Guide to Molecular Cloning Techniques, Methods in Enzyiology, Vol. 152, Academic Press, Inc., San Diego, Calif. (“Berger”); Sambrook et al., Molecular Cloning—A Laboratory Manual (2nd Ed.), Vols. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”). Examples of techniques sufficient to direct persons of ordinary skill in the art through in vitro amplification methods, including the polymerase chain reaction (PCR) the ligase chain reaction (LCR), Qp-replicase amplification and other RNA polymerase mediated techniques (e.g., NASBA), e.g., for the production of the homologous nucleic acids of the invention are found in Berger, Sambrook, and Ausubel, as well as Mullis et al. (1987) U.S. Pat. No. 4,683,202; PCR Protocols A Guide to Methods and Applications (Innis et al. eds.) Academic Press Inc. San Diego, Calif. (1990) (Innis); Arnheim & Levinson (Oct. 1, 1990) C&EN 36-47; The Journal Of NIH Research (1991)3:81-94; (Kwoh et al. (1989) Proc. Nat'l Acad. Sci. USA 86: 1173; Guatelli et al. (1990) Proc. Nat'l Acad. Sci. USA 87:1874; Lomell et al. (1989) J. Clin. Chem. 35:1826; Landegren et al. (1988) Science 241:1077-1080; Van Brunt (1990) Biotechnology 8:291-294; Wu and Wallace (1989) Gene 4:560; Barringer et al. (1990) Gene 89:117, and Sooknanan and Malek (1995) Biotechnology 13:563-564. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369:684-685 and the references therein, in which PCR amplicons of up to 40kb are generated. One of ordinary skill in the art will appreciate that essentially any RNA can be converted into a double stranded DNA suitable for restriction digestion, PCR expansion and sequencing using reverse transcriptase and a polymerase. See Ausubel, Sambrook and Berger, all supra.

The present invention also relates to host cells that are transduced with vectors of the invention, and the production of polypeptides of the invention by recombinant techniques. Host cells are genetically engineered (i.e., transduced, transformed or transfected) with the vectors of this invention, which may be, for example, a cloning vector or an expression vector. The vector may be, for example, in the form of a plasmid, a viral particle, a phage, etc., The engineered host cells can be cultured in conventional nutrient media modified as appropriate for activating promoters, selecting transformants, or amplifying the env gene. The culture conditions, such as temperature, pH and the like, are those previously used with the host cell selected for expression, and will be apparent to those skilled in the art and in the references cited herein, including, e.g., Freshney (1994) Culture of Animal Cells, A Manual of Basic Technique, 3d ed., Wiley-Liss, New York and the references cited therein.

The envelope proteins of the invention (or fragments thereof) can also be produced in non-animal cells such as plants, yeast, fungi, bacteria and the like. In addition to Sambrook, Berger and Ausubel, details regarding cell culture can be found in Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg New York) and Atlas and Parks (eds.) The Handbook of Microbiological Media (1993) CRC Press, Boca Raton, Fla.

The polynucleotides of the present invention may be included in any one of a variety of expression vectors for expressing a polypeptide. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, pseudorabies, adenovirus, adeno-associated virus, retroviruses and many others. Any vector that transduces genetic material into a cell, and, if replication is desired, that is replicable and viable in the relevant host can be used.

The nucleic acid sequence in the expression vector is operatively linked to an appropriate transcription control sequence (promoter) to direct mRNA synthesis. Examples of such promoters include: LTR or SV40 promoter, E. coli lac or trp promoter, phage lambda P_{L} promoter, and other promoters known to control expression of genes in prokary otic or eukaryotic cells or their viruses. The expression vector also contains a ribosome binding site for translation initiation, and a transcription terminator. The vector optionally includes appropriate sequences for amplifying expression. In addition, the expression vectors optionally comprise one or more selectable marker genes to provide a phenotypic trait for selection of transformed host cells, such as dihydrofolate reductase or neomycin resistance for eukaryotic cell culture, or such as tetracycline or ampicillin resistance in E. coli.

The vector containing the appropriate DNA sequence as described above, as well as an appropriate promoter or control sequence, may be employed to transform an appropriate host to permit the host to express the protein. Examples of appropriate expression hosts include: bacterial cells, such as E. coli, Streptomyces, and Salmonella typhimurium; fungal cells, such as Saccharomyces cerevisiae, Pichia pastoris, and Neurospora crassa; insect cells such as Drosophila and Spodoptera frugiperda; mammalian cells such as 293T, CHO, COS, BHK, HEK or Bowes melanoma; plant cells, etc. It is understood that not all cells or cell lines need to be capable of producing fully functional envelope proteins; for example, antigenic fragments of an envelope protein may be produced in a bacterial or other expression system. The invention is not limited by the host cells employed.

In bacterial systems, a number of expression vectors may be selected depending upon the use intended for the envelope protein. For example, when large quantities of envelope proteins or fragments thereof are needed for the induction of antibodies, vectors that direct high level expression of fusion proteins that are readily purified may be desirable. Such vectors include, but are not limited to, multifunctional E. coli cloning and expression vectors such as BLUESCRIPT (Stratagene), in which the envelope protein coding sequence may be ligated into the vector in-frame with sequences for the amino-terminal (N-terminal) Met and the subsequent 7 residues of beta-galactosidase so that a hybrid protein is produced; pIN vectors (Van Heeke & Schuster (1989) J. Biol. Chem. 264:5503-5509); pET vectors (Novagen, Madison Wis.); and the like.

Similarly, in the yeast Saccharomyces cerevisiae a number of vectors containing constitutive or inducible promoters such as alpha factor, alcohol oxidase and PGH may be used for production of the envelope proteins of the invention. For reviews, see Ausubel et al. (supra); and Grant et al. (1987) Methods in Enzymologv 153:516-544.

In mammalian host cells, a number expression systems, such as viral-based systems, may be utilized. In cases where an adenovirus is used as an expression vector, a coding sequence is optionally ligated into an adenovirus transcription/translation complex consisting of the late promoter and tripartite leader sequence. Insertion in a nonessential E1 or E3 region of the viral genome will result in a viable virus capable of expressing the envelope protein in infected host cells (Logan and Shenk (1984) Proc. Nat'l Acad. Sci. 81:3655-3659)). Transcription enhancers, such as the rous sarcoma virus (RSV) enhancer, may also be used to increase expression in mammalian host cells.

Additional Expression Elements

Specific initiation signals can aid in efficient translation of an envelope protein polypeptide coding sequence. These signals can include, e.g., the ATG initiation codon and adjacent sequences. In cases where an envelope protein polypeptide coding sequence, its initiation codon and upstream sequences are inserted into the appropriate expression vector, no additional translational control signals may be needed. However, in cases where only coding sequence (e.g., a mature protein coding sequence), or a portion thereof, is inserted, exogenous transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression may be enhanced by the inclusion of enhancers appropriate to the cell system in use (Scharf, D. et al. (1994) Results Probl. Cell Differ. 20:125-62; Bittner et al. (1987) Methods in Enzymol. 153:516-544).

Secretion/Localization Sequences

Polynucleotides of the invention can also be fused, for example, in-frame to nucleic acid encoding a secretion/localization sequence, to target polypeptide expression to a desired cellular compartment, membrane, or organelle, or to direct polypeptide secretion to the periplasmic space or into the cell culture media. Such sequences are known to those of ordinary skill, and include secretion leader peptides, organelle targeting sequences (e.g., nuclear localization sequences, ER retention signals, mitochondrial transit sequences, chloroplast transit sequences), membrane localization/anchor sequences (e.g., stop transfer sequences, GPI anchor sequences), and the like.

Expression Hosts

In a further embodiment, the present invention relates to host cells containing the above-described constructs. The host cell can be a eukaryotic cell, such as a mammalian cell, a yeast cell, or a plant cell, or the host cell can be a prokaryotic cell, such as a bacterial cell. Introduction of the construct into the host cell can be effected by calcium phosphate transfection, DEAE-Dextran mediated transfection, electroporation, or other common techniques (Davis, L., Dibner, M., and Battey, I. (1986) Basic Methods in Molecular Biology).

A host cell strain is optionally chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing that cleaves a “pre” or a “prepro” form of the protein may also be important for correct insertion, folding and/or function. Different host cells such as 293T, CHO, HeLa, BHK, MDCK, WI38, etc., have specific cellular machinery and characteristic mechanisms for such post-translational activities and may be chosen to ensure the correct modification and processing of the introduced, foreign protein.

For long-term, high-yield production of recombinant proteins, stable expression can be used. For example, cell lines that stably express a polypeptide of the invention are transduced using expression vectors that contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. For example, resistant clumps of stably transformed cells can be proliferated using tissue culture techniques appropriate to the cell type.

Host cells transformed with a nucleotide sequence encoding a polypeptide of the invention are optionally cultured under conditions suitable for the expression and recovery of the encoded protein from cell culture. The protein or fragment thereof produced by a recombinant cell may be secreted, membrane-bound, or contained intracellularly, depending on the sequence and/or the vector used. As will be understood by those of ordinary skill in the art, expression vectors containing polynucleotides encoding the retrovirus envelope proteins of the invention can be designed with signal sequences that direct secretion of the mature polypeptides through a prokaryotic or eukaryotic cell membrane.

The present invention also includes at least one polynucleotide consensus sequence derived from a comparison of two or more stress or shear resistant polynucleotide sequences described herein. A polynucleotide consensus sequence as used herein means a nonnaturally-occurring or recombinant polynucleotide sequence that predominantly includes those nucleic acid residues that are common to all isolated or recombinant (or modified) polynucleotides of the present invention described herein and that includes, at one or more of those positions wherein there is no nucleic acid residue common to all subtypes, a nucleic acid residue that predominantly occurs at that position and in no event includes any nucleic acid residue which is not extant in that position in at least one isolated or recombinant (or modified) polynucleotide of the invention.

Additional Sequences

The polynucleotides of the present invention may also comprise a coding sequence fused in-frame to a marker sequence which, e.g., facilitates purification of the encoded polypeptide. Such purification facilitating domains include, but are not limited to, metal chelating peptides such as histidine-tryptophan modules that allow purification on immobilized metals, a sequence that binds glutathione (e.g., GST), a hemagglutinin (HA) tag (corresponding to an epitope derived from the influenza hemagglutinin protein; Wilson et al. (1984) Cell 37:767), maltose binding protein sequences, the FLAG epitope utilized in the FLAGS extension/affinity purification system (Immunex Corp., Seattle, WA), and the like. The inclusion of a protease-cleavable polypeptide linker sequence between the purification domain and the envelope protein sequence is useful to facilitate purification.

One expression vector contemplated for use in the compositions and methods described herein provides for expression of a fusion protein comprising a polypeptide of the invention fused to a polyhistidine region separated by an enterokinase cleavage site. The histidine residues facilitate purification on IMIAC (immobilized metal ion affinity chromatography, as described in Porath et al. (1992) Protein Expression and Purification 3:263-281), while the enterokinase cleavage site provides a means for separating the envelope protein polypeptide from the fusion protein. pGEX vectors (Promega; Madison, Wis.) can also be used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

Polypeptide Production and Recovery

Following transduction of a suitable host strain and growth of the host strain to an appropriate cell density, the selected promoter is induced by appropriate means (e.g., temperature shift or chemical induction)-and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents, or other methods, which are well known to those of ordinary skill in the art.

As noted, many references are available for the culture and production of many cells, including cells of bacterial, plant, animal (especially mammalian) and archebacterial origin. See, e.g., Sambrook, Ausubel, and Berger (all supra), as well as Freshney (1994) Culture of Animal Cells, a Manual of Basic Technique, 3d ed., Wiley-Liss, New York and the references cited therein; Doyle and Griffiths (1997) Mammalian Cell Culture: Essential Techniques John Wiley and Sons, NY; Humason (1979) Animal Tissue Techniques, 4^{th} ed., W. H. Freeman and Company; and Ricciardelli et al., (1989) In vitro Cell Dev. Biol. 25:1016-1024. For plant cell culture and regeneration, Payne et al. (1992) Plant Cell and Tissue Culture in Liquid Systems, John Wiley & Sons, Inc. New York, NY; Gamborg and Phillips (eds.) (1995) Plant Cell, Tissue and Organ Culture; Fundamental Methods Springer Lab Manual, Springer-Verlag (Berlin Heidelberg New York) and Plant Molecular Biology (1993) R. R. D. Croy, Ed. Bios Scientific Publishers, Oxford, U.K. ISBN 0 12 198370 6. Cell culture media in general are set forth in Atlas and Parks (eds.) The Handbook of Microbiological Media (1993) CRC Press, Boca Raton, Fla. Additional information for cell culture is found in available commercial literature such as the Life Science Research Cell Culture Catalogue (1998) from Sigma-Aldrich, Inc. (St. Louis, Mo.) (“Sigma-LSRCCC”) and, e.g., the Plant Culture Catalogue and supplement (1997) also from Sigma-Aldrich, Inc. (St. Louis, Mo.) (“Sigma-PCCS”).

Polypeptides of the invention can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxylapatite chromatography, and lectin chromatography. Protein refolding steps can be used, as desired, in completing configuration of the mature protein. Finally, high performance liquid chromatography (HPLC) can be employed in the final purification steps. In addition to the references noted supra, a variety of purification methods are well known in the art, including, e.g., those set forth in Sandana (1997) Bioseparation of Proteins, Academic Press, Inc.; and Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, N.Y.; Janson and Ryden (1998) Protein Purification: Principles, High Resolution Methods and Aplications, Second Edition Wiley-VCH, NY; and Walker (1998) Protein Protocols on CD-ROM Humana Press, NJ.

In vitro Expression Systems

Cell-free transcription/translation systems can also be employed to produce polypeptides using DNAs or RNAs of the present invention. Several such systems are commercially available. A general guide to in vitro transcription and translation protocols is found in Tymms (1995) In vitro Transcription and Translation Protocols: Methods in Molecular Biology Vol. 37, Garland Publishing, NY.

Modified Amino Acids

Polypeptides of the invention may contain one or more modified amino acids. The presence of modified amino acids may be advantageous in, for example, (a) increasing polypeptide serum half-life, (b) reducing polypeptide antigenicity, and/or (c) increasing polypeptide storage stability. Amino acid(s) are modified, for example, co-translationally or post-translationally during recombinant production (e.g., N-linked glycosylation at N-X-S/T motifs during expression in mammalian cells) or modified by synthetic means.

Non-limiting examples of a modified amino acid include a glycosylated amino acid, a sulfated amino acid, a prenlyated (e.g., farnesylated, geranylgeranylated) amino acid, an acetylated amino acid, an acylated amino acid, a PEG-ylated amino acid, a biotinylated amino acid, a carboxylated amino acid, a phosphorylated amino acid, and the like. References adequate to guide one of ordinary skill in the art in the modification of amino acids are replete throughout the literature. Example protocols are found in Walker (1998) Protein Protocols on CD-ROM, Human Press, Towata, N.J.

USES OF POLYNUCLEOTIDES AND POLYPEPTIDES OF THE INVENTION

The stress or shear resistant polynucleotides of the invention have a variety of uses in, for example: recombinant production (i.e., expression) of the recombinant or modified envelope protein polypeptides of the invention or retroviruses of the invention, including for those applications in which improved manufacturability and improved manufacturing yield compared to known retroviruses and retroviral vectors is desired is desired; as therapeutics or prophylactics in methods to treat or prevent diseases; in gene therapy methods, including, e.g., retroviral applications for use in vivo human gene therapy methods, as immunogens; as diagnostic probes for the presence of complementary or partially complementary nucleic acids (including for detection of natural retroviral coding nucleic acids); as substrates for further reactions, e.g., PCR or cloning, e.g., digestion, ligation, reactions to produce new and/or improved envelope proteins, and the like. Polynucleotides that encode an envelope protein of the invention (e.g., having stress or sheer resistant properties, as demonstrated by, e.g., an ability to withstand one or more rounds of ultracentrifugation over various periods of time), or complements of the polynucleotides, are optionally administered to a cell or organism to accomplish a therapeutically or prophylactically useful process or to express a therapeutically or prophylactically useful product in vivo, ex vivo, or in vitro. These applications, including in vivo or ex vivo applications, such as gene therapy, include a multitude of techniques by which gene expression may be altered in cells. Such methods include, for example, infecting with a retrovirus comprising the polynucleotides and/or polypeptides of the invention. Optionally, the retrovirus, further comprises additional exogenous, e.g., therapeutic or prophylactic gene construct, sequences. The stress or shear resistant polynucleotides of the invention are also useful in applications in which consistent purification and concentration of retroviral vector products is desired, efficient manufacturability of stable retroviral vectors stocks of high titer is desired, and/or high manufacturing yield is desired, despite, e.g., severe processing conditions, ultrapurification, ultrafiltration, or ultracentrifugation.

Polypeptide Expression

Polynucleotides encoding envelope protein polypeptides of the invention are particularly useful for in vivo and ex vivo therapeutic or prophylactic applications, using techniques well known to those skilled in the art. For example, cultured cells are engineered ex vivo with a polynucleotide (DNA or RNA), with the engineered cells then being returned to the patient. Cells may also be engineered in vivo for expression of a polypeptide in vivo. Polynucleotides encoding envelope protein polypeptides of the invention are also useful for in vitro applications.

A number of viral vectors suitable for organismal in vivo transduction and expression are known. Such vectors include retroviral vectors (see Miller (1992) Curr. Top. Microbiol. Immunol. 158:1-24; Salmons and Gunzburg (1993) Human Gene Therapy 4:129-141; Miller et al. (1994) Meth. Enzymol. 217:581-599) and adeno-associated vectors (reviewed in Carter (1992) Curr. Opinion Biotech. 3:533-539; Muzcyzka (1992) Curr. Top. Microbiol. Immunol. 158:97-129). Other viral vectors that are used include adenoviral vectors, herpes viral vectors and Sindbis viral vectors, as generally described in, e.g., Jolly (1994) Cancer Gene Ther. 1:51-64; Latchman (1994) Molec. Biotechnol. 2:179-195; Johanning et al. (1995) Nucl. Acids Res. 23:1495-1501.

Gene therapy provides methods for combating chronic infectious diseases (e.g., HIV infection, viral hepatitis (e.g., hepatitis B, C and A)), as well as non-infectious diseases including cancer and some forms of congenital defects such as enzyme deficiencies. Several approaches for introducing nucleic acids into organisms or cells in vivo, ex vivo and in vitro have been used. These include liposome based gene delivery (Debs and Zhu (1993) WO 93/24640 and U.S. Pat. No. 5,641,662; Mannino and Gould-Fogerite (1988) BioTechniques 6(7):682-691; Rose, U.S. Pat No. 5,279,833; Brigham (1991) WO 91/06309; and Felgner et al. (1987) Proc. Nat'l Acad. Sci. USA 84:7413-7414); Brigham et al. (1989) Am. J. Med. Sci., 298:278-281; Nabel et al. (1990) Science 249:1285-1288; Hazinski et al. (1991) Am. J. Resp. Cell Molec. Biol. 4:206-209; and Wang and Huang (1987) Proc. Nat'l Acad. Sci. USA 84:7851-7855).; adenoviral vector mediated gene delivery, e.g., to treat cancer (see, e.g., Chen et al. (1994) Proc. Nat'l Acad. Sci. USA 91:3054-3057; Tong et al. (1996) Gynecol. Oncol. 61:175-179; Clayman et al. (1995) Cancer Res. 5:1-6; O'Malley et al. (1995) Cancer Res. 55:1080-1085; Hwang et al. (1995) Am. J. Respir. Cell Mol. Biol. 13:7-16; Haddada et al. (1995) Curr. Top. Microbiol. Immunol. 199 (Pt. 3):297-306; Addison et al. (1995) Proc. Nat'l Acad. Sci. USA 92:8522-8526; Colak et al. (1995) Brain Res. 691:76-82; Crystal (1995) Science 270:404-410; Elshami et al. (1996) Human Gene Ther. 7:141-148; Vincent et al. (1996) J. Neurosurg. 85:648-654); and many other diseases. Replication-defective retroviral vectors harboring therapeutic or prophylactic polynucleotide sequence as part of the retroviral genome have also been used, particularly with regard to simple MLV vectors. See, e.g., Miller et al. (1990) Mol. Cell. Biol. 10:4239 (1990); Kolberg (1992) J. NIH Res. 4:43, and Cometta et al. (1991) Hum. Gene Ther. 2:215). Nucleic acid transport coupled to ligand-specific, cation-based transport systems (Wu and Wu (1988) J. Biol. Chem. 263:14621-14624), has also been used. Naked DNA expression vectors have also been described (Nabel et al. (1990), supra); Wolff et al. (1990) Science 247:1465-1468). In general, these approaches can be adapted to the invention by incorporating nucleic acids encoding the stress (and/or shear) resistant envelope proteins or fragments thereof described herein into the appropriate vectors.

General texts that describe gene therapy protocols, which can be adapted to the present invention by introducing the nucleic acids of the invention into patients, include Robbins (1996) Gene Therapy Protocols, Humana Press, NJ, and Joyner (1993) Gene Targeting: A Practical Approach, IRL Press, Oxford, England.

Antisense Technology

In addition to expression of the nucleic acids of the invention as gene replacement nucleic acids, the nucleic acids are also useful for sense and anti-sense suppression of expression, e.g., to down-regulate expression of a nucleic acid of the invention, once expression of the nucleic acid is no-longer desired in the cell. Similarly, the nucleic acids of the invention, or subsequences or anti-sense sequences thereof, can also be used to block expression of naturally occurring homologous nucleic acids. A variety of sense and anti-sense technologies are known in the art, e.g., as set forth in Lichtenstein and Nellen (1997) Antisense Technology: A Practical Approach IRL Press at Oxford University, Oxford, England, and in Agrawal (1996) Antisense Therapeutics Humana Press, NJ, and the references cited therein.

Use as Probes

Also contemplated are uses of polynucleotides, also referred to herein as oligonucleotides, typically having at least 12 bases, preferably at least 15, more preferably at least 20, 30, or 50 bases, which hybridize under at least stringent, highly stringent conditions, ultra-high stringency conditions, or ultra-ultra-high stringency conditions to an envelope protein polynucleotide sequence described above. The polynucleotides may be used as probes, primers, sense and antisense agents, and the like, according to methods as noted supra and infra.

OTHER POLYNUCLEOTIDE COMPOSITIONS

The invention also includes compositions comprising two or more polynucleotides of the invention (e.g., as substrates for recombination) that encode stress or shear resistant polypeptides. The composition can comprise a library of recombinant nucleic acids of the invention encoding stress or shear resistant polypeptides, where the library contains at least 2, 3, 5, 10, 20, or 50 or more of said nucleic acids. The nucleic acids are optionally cloned into expression vectors, providing expression libraries.

The invention also includes compositions produced by digesting one or more stress or shear resistant polynucleotides of the invention with a restriction endonuclease, an RNAse, or a DNAse (e.g., as is performed in certain processes related to the cloning of the nucleic acids, including those of the invention).

Also included in the invention are compositions produced by incubating one or more of the fragmented nucleic acid sets in the presence of ribonucleotide- or deoxyribonucleotide triphosphates and a nucleic acid polymerase. The nucleic acid polymerase may be an RNA polymerase, a DNA polymerase, or an RNA-directed DNA polymerase (e.g., a “reverse transcriptase”); the polymerase can be, e.g., a thermostable DNA polymerase (such as VENT, TAQ, or the like).

RETROVIRUS ENVELOPE PROTEIN POLYPEPTIDES

The invention provides novel isolated or recombinant retrovirus envelope protein polypeptides, fragments of envelope protein polypeptides, related fusion polypeptides or proteins, or functional equivalents thereof, which are collectively referred to herein as “retrovirus envelope protein polypeptides” or “retrovirus envelope proteins” or, simply, “envelope protein polypeptides or “envelope proteins.”

Some such retrovirus envelope protein polypeptides of the invention exhibit stress and/or shear resistant properties as described herein, and such polypeptides are referred to collectively as “stress resistant retrovirus envelope proteins” or “shear resistant retrovirus envelope proteins.” A polypeptide of the invention or fragment thereof that confers stress and/or shear resistant properties on the retrovirus of which the polypeptide is an envelope protein. Such retrovirus envelope protein polypeptides, fragments of envelope protein polypeptides, related fusion polypeptides or proteins, or functional equivalents thereof exhibit stress and/or shear resistant properties.

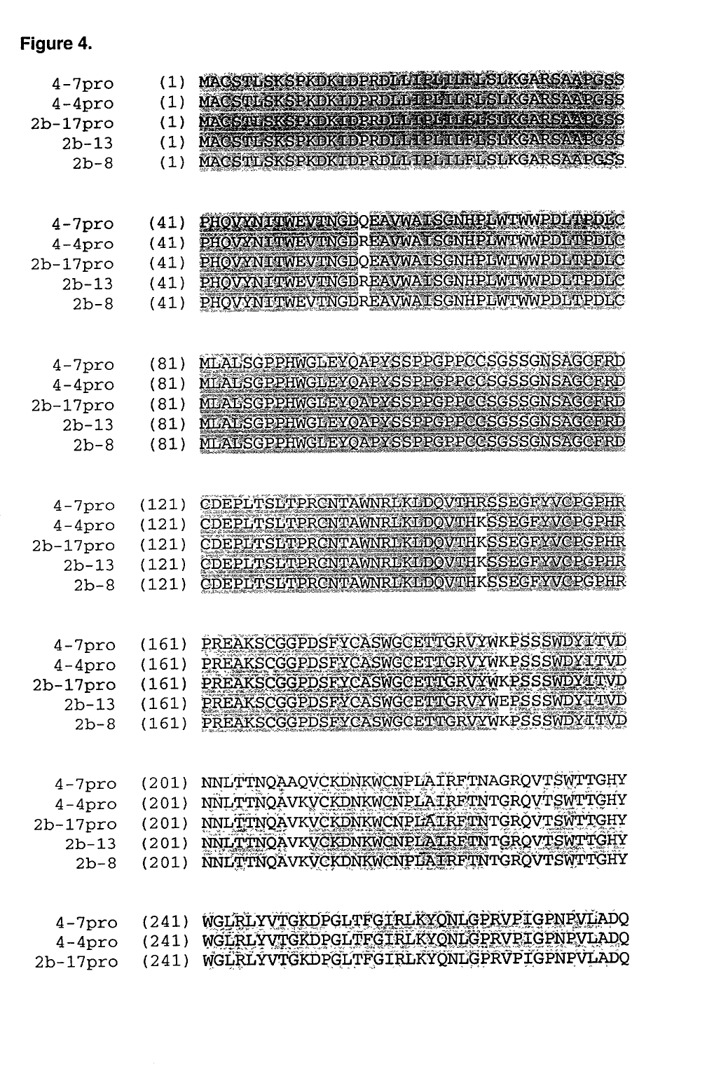

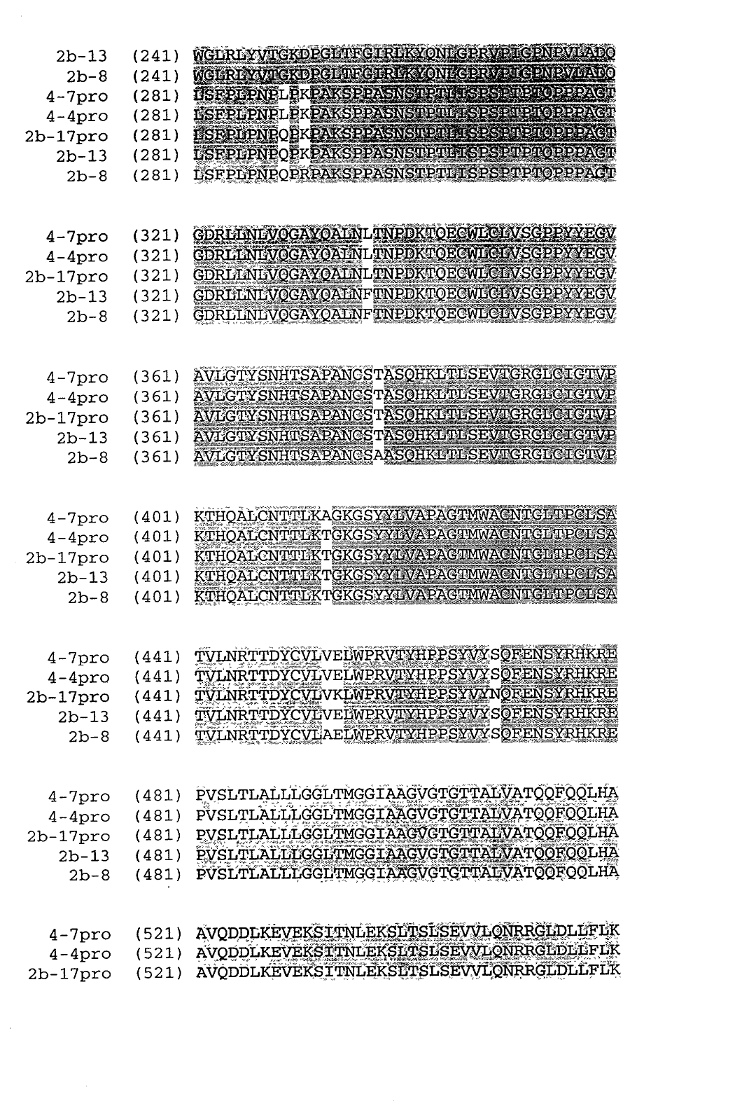

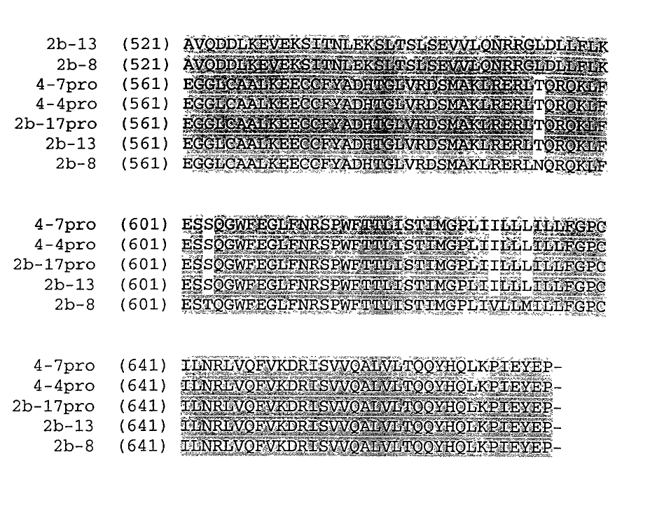

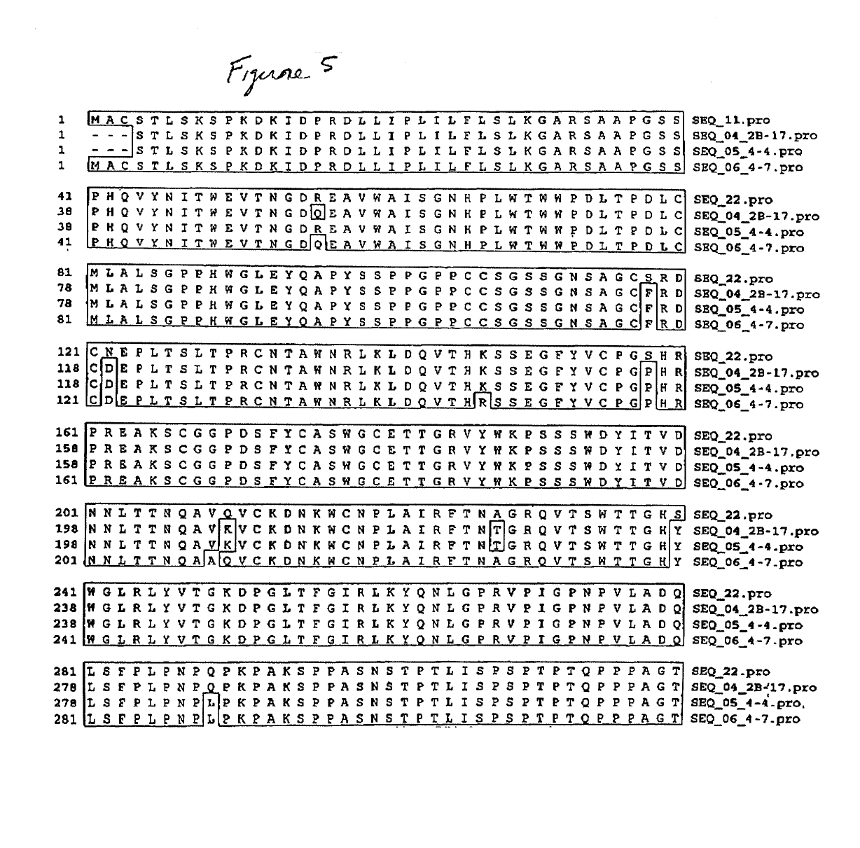

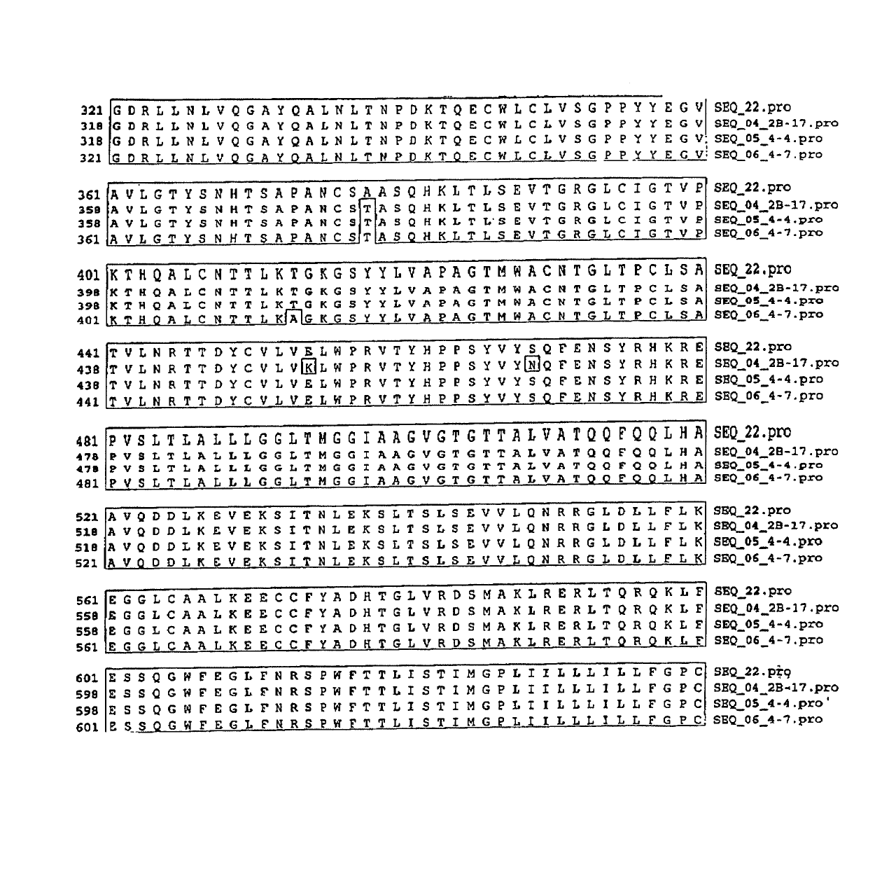

We generated five novel retroviral envelope proteins, corresponding to clones 4-4, 4-7, 2B-17, 2B-13, and 2B-8. The amino acid sequences and corresponding nucleic acid sequences for each of these envelope proteins are an aspect of the invention. FIG. 4 shows an amino acid sequence alignment of the amino acid sequences of SEQ ID NO:4 (2B-17), SEQ ID NO:5 (4-4), SEQ ID NO:6 (4-7), SEQ ID NO:7 (2B-13) and SEQ ID NO:8 (2B-8). The nucleotide sequences of these clones, SEQ ID NO:1 (2B-17), SEQ ID NO:2 (44), SEQ ID NO:3 (4-7), SEQ ID NO:9 (2B-13) and SEQ ID NO:10 (2B-8), can be similarly aligned.

The envelope protein polypeptides corresponding to clone 2B-17, 4-4, and 4-7, respectively, exhibit stress and/or shear resistant properties and are capable of withstanding stress forces typically experienced during purification and concentration procedures (e.g., ultracentrifugation). For example, with a stress or shear resistant retrovirus envelope protein polypeptide corresponding to clone 2B-17, 4-4, or 4-7 incorporated into a retrovirus as an envelope protein, the retrovirus is capable of withstanding ultracentrifugation conducted at a force of at least about 90,000×g (gravity), 100,000×g, 110,000×g, or 120,000×g or more for at least about 20, 30, 40, 50, 55, 60, 70, 75, 80, 85, or 90 minutes or more, while maintaining or preserving an infectious titer level of at least about 10%, 20%, 30%, 40%, 50%, 60%, 70%, 75%, 80%, 85%, 90%, 91,%, 92%, 93%, 94%, 95%, 96%, 97%, 98%, 99% or 100% of the infectious titer level of an identical retrovirus comprising an identical stress and/or shear resistant retrovirus envelope protein polypeptide (or fragment thereof having such property) as an envelope protein that has not been subjected to such ultracentrifugation. Such a polypeptide of the invention is capable of withstanding ultracentrifugation under such conditions. In contrast, the envelope proteins corresponding to clones 2B-8 and 2B-13, while comprising novel retroviral sequences, are sensitive to such ultracentrifugation procedures.

Preferentially, in some embodiments, the retrovirus comprising a stress and/or shear resistant polypeptide (or fragment thereof) is capable of withstanding ultracentrifugation of a force in excess of at least about 120,000×g for at least about 50, 55, 60, 61, 62, 63, 64, 65, 68, 69, 70, or 80 minutes or more, such that it has, retains, or preserves an infectious titer level of at least about 50%, 60%, 70%, 80%, 85%, 90%, 91%, 92%, 93%, 94%, 95%, 96%, 97%, 98%, 99%, or 100% relative to the infectious titer level of an identical retrovirus comprising the stress and/or shear resistant retrovirus envelope protein polypeptide (or fragment thereof) that has not been subjected to ultracentrifugation. See, e.g., FIG. 3.

As noted above, stress (and/or shear) resistant retroviral clones of the invention include recombinant stress (and/or shear) resistant retrovirus envelope protein polypeptides of the invention (or fragments thereof) as envelope proteins (e.g., SEQ ID NOS:4-6). An isolated, modified or recombinant envelope protein of the invention includes a polypeptide comprising a sequence selected from SEQ ID NOS:4-8, and conservatively modified variations thereof.

Further, the invention provides nucleic acids that encode recombinant stress (and/or shear) resistant retrovirus envelope protein polypeptides that conferred stress (and/or shear) resistant properties on the retroviruses (e.g., SEQ ID NOS:1-3).

In one aspect, the invention provides a stress or shear resistant retrovirus envelope polypeptide comprising an amino acid sequence that has at least about 80, 85, 90, 95, 96, 97, 98, 99% or more amino acid sequence identity with an amino acid sequ~ence of at least one of SEQ ID NOS:4-6. Some such stress or shear resistant retrovirus envelope polypeptide of claim 76, wherein said polypeptide is capable of withstanding ultracentrifugation at a force of at least about 120,000×g for at least about 30 minutes. For some such stress or shear resistant retrovirus envelope polypeptides, the amino acid sequence is not encoded by a polynucleotide sequence represented by any of GenBank Nucleic Acid Accession Nos. Z35109, K02714, Y13893, D88386, Z11128, M93134, X02794, AF288940, U94692, AF030173, S80869, S77017, S77015, S77012, J01998, AF169256, and AH000833. In a preferred embodiment, some such stress or shear resistant retrovirus envelope-polypeptides comprise an amino acid sequence that has at least about 96% 97%, 98%, or 99% amino acid sequence identity with an amino acid sequence of at least one of SEQ ID NOS:4-6.

That such a polypeptide is stress of shear resistant is determined as described in the Examples below by making a retrovirus comprising said stress or shear resistant polypeptide (or nucleic acid encoded therein) and subjecting the retrovirus to ultracentrifugation conditions. Retroviruses that survive ultracentrifugation at a force of least about or in excess of 120,000× gravity (g) for at least about 20, 30, 40, 50, 60, 68, 70, 80, or 90 minutes or more can be selected and isolated. Further, stress or shear resistant retroviruses (or, e.g., supernatants comprising such retroviruses) that maintain an infectious titer level of at least about 25%, 30%, 40%, 50%, 60%, 70%, 80%, 85%, 90%, 91%, 92%, 93%, 94%, 95%, 96%, 97%, 98%, 98.5%, 99%, 99.5%, or 100% of the infectious titer level of an identical retrovirus comprising the same stress or shear resistant envelope protein polypeptide (or, e.g., supernatant comprising such retrovirus) that has not undergone such ultracentrifugation under such conditions for such time period.

The invention also includes an isolated or recombinant polypeptide which comprises an amino acid sequence encoded by a coding polynucleotide sequence, the coding polynucleotide sequence selected from the group of: (a) a polynucleotide sequence selected from at least one of SEQ ID NOS:1-3, or a complementary nucleic acid sequence thereof; (b) a polynucleotide sequence that encodes a polypeptide selected from any of SEQ ID NOS:4-6; (c) a polynucleotide sequence which hybridizes under stringent conditions over substantially the entire length of a polynucleotide sequence (a) or (b); (d) a polynucleotide sequence encoding a polypeptide, the polypeptide comprising an amino acid sequence which is substantially identical over at least about 550, 600, or 650 contiguous amino acid residues of any one of SEQ ID NOS:4-6; provided said amino acid sequence is not a protein sequence encoded by a nucleotide sequence represented by any of GenBank Accession Nos.,Z35109, K02714, Y13893, D88386, Z11128, M93134, X02794, AF288940, U94692, AF030173, S80869, S77017, S77015, S77012, J01998, AF169256, and AH000833; and (e) a polynucleotide sequence encoding a stress or shear resistant retrovirus envelope polypeptide, which polynucleotide sequence has at least about 95% identity to at least one polynucleotide sequence of (a), (b), (c), or (d). Some such isolated or recombinant polypeptides are capable of withstanding centrifugation at a force of at least about 120,000×g for at least about 30 minutes.

Alignment of both the DNA and protein sequences of the recombinant stress (and/or shear) resistant clones 4-4, 4-7, and 2B-17 with DNA and protein sequences of sensitive clones 2B-13 and 2B-8, respectively, revealed the differences between their respective nucleotide and amino acid sequences. Such differences in nucleotide and amino acid sequences between the stress resistant and sensitive clones provide information about those nucleotides and amino acid residues that may contribute to the stress and/or shear resistant phenotype.

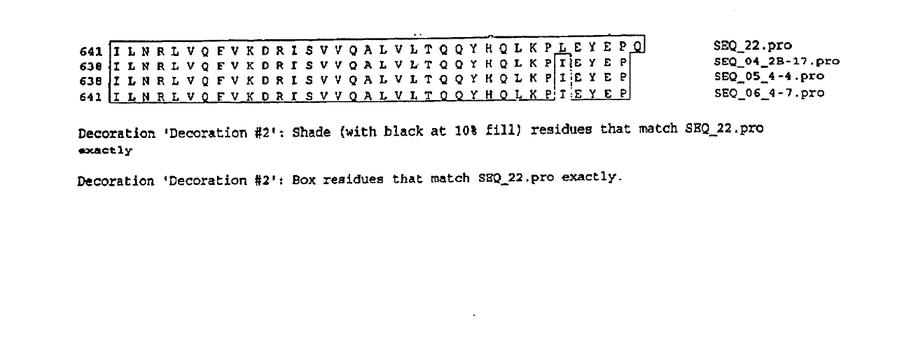

A “variant polypeptide” is a polypeptide that differs in one or more amino acid residues from a parent polypeptide, typically in at least 1, 2, 3, 4, 5, 6, 7, 8, 9, 10, 11, 12, 13, 14 or 15 amino acid residues. In one aspect, the invention provides an isolated, modified or recombinant variant retrovirus polypeptide of a parent retrovirus polypeptide, the variant polypeptide comprising at least one substitution from the group of Q454K and S469N, wherein the parent retrovirus polypeptide comprises amino acid residues 4-615 of the Friend murine leukemia virus polypeptide shown in SEQ ID NO:11 (GenBank Acc. No. CAA84492). See, e.g., FIG. 5. As would be readily apparent to one of ordinary skill in the art, the term “Q454K” refers to the substitution at amino acid residue 454 in SEQ ID NO:11 of K (lysine) for Q (glutamine). Some such variant polypeptides further comprises at least one additional substitution selected from the group of R56Q, K147R, V209A, Q210K, A228T, Q289L, A378T, and T413A. For other such variant polypeptides, the parent polypeptide comprises amino acid residues 4-671 of SEQ ID NO:11.

The invention also provides isolated, modified or recombinant retrovirus variant polypeptides that differ in one or more amino acid residues from a known parent polypeptide (e.g., typically in at least 1, 2, 3, 4, 5, 6, 7, 8, 9, 10, 11, 12, 13, 14 or 15 amino acid residues), wherein the known parent polypeptide is not the polypeptide represented by SEQ I) NO:11. In addition, the invention provides isolated, modified or recombinant retrovirus variant polypeptides that differ in one or more amino acid residues (e.g., typically in at least 1, 2, 3, 4, 5, 6, 7, 8, 9, 10, 11, 12, 13, 14 or 15 amino acid residues) from one of the parent retrovirus polypeptides from which the stress or shear resistant envelope protein polypeptides of the invention were derived or from one of the isolated, modified or recombinant sensitive retrovirus proteins described herein (e.g., SEQ ID NOS:7-8).

A “variant polynucleotide” is a polynucleotide that differs in one or more nucleic acid residues from a parent polynucleotide, typically in at least 1, 2, 3, 4, 5, 6, 7, 8, 9, 10, 11, 12, 13, 14, 15, 20, 25, 30, 33, 36,40,42, or45 nucleic acid residues. The invention includes isolated, modified or recombinant retrovirus variant polynucleotides that differ in one or more amino acid residues from a known parent retroviral polynucleotide, from a polynucleotide encoding a polypeptide comprising an isolated, modified or recombinant sensitive retrovirus proteins described herein (e.g., SEQ ID NOS:7-8), or from a parent retroviral polypeptide from which the stress or shear resistant envelope protein polypeptides of the invention were derived.

Without being bound by theory, the increased resistance of retroviral envelope proteins to ultracentrifugation as described herein may be accounted for, at least in part, by decreased dissociation of the SU and TM subunits (McGrath et al. (1978) J. Virol. 25:923), which are associated via a disulfide bond. Mutations have been identified in SU that apparently destabilize the SU-TM interaction (Gray et al. (1993) J. Virol. 67:3489-3496), but no mutations have been reported that stabilize the interaction. The likely mechanism for the enhanced stress (and/or shear) resistance of the clones isolated in this study is stabilization of the SU-TM interaction.

In yet another aspect, the invention provides an isolated, modified or recombinant polypeptide which comprises an amino acid sequence encoded by a coding polynucleotide sequence, the coding polynucleotide sequence selected from the group of: (a) a polynucleotide sequence selected from at least one of SEQ ID NOS:9-10; (b) a polynucleotide sequence that encodes a polypeptide selected from any of SEQ ID NOS:7-8; (c) a polynucleotide sequence which hybridizes under stringent conditions over substantially the entire length of a polynucleotide sequence (a) or (b); (d) a polynucleotide sequence encoding a polypeptide, the polypeptide comprising an amino acid sequence which is substantially identical over at least about 550, 600, or 650 contiguous amino acid residues of any one of SEQ ID NOS:7-8; provided said amino acid sequence is not a protein sequence encoded by a nucleotide sequence represented by any of GenBank Accession Nos. Z35109, K02714, Y13893, D88386, ZI 1128, M93134, X02794, AF288940, U94692, AF030173, S80869, S77017, S77015, S77012, J01998, AF169256, and AH000833; and (e) a polynucleotide sequence encoding a stress or shear resistant retrovirus envelope polypeptide, which polynucleotide sequence has at least about 95% identity to at least one polynucleotide sequence of (a), (b), (c), or (d).

The invention also includes fusion proteins comprising all such polypeptides of the invention described herein with at least one additional amino acid sequence. In addition, the invention provides compositions comprising at least one polypeptide of the invention described herein and a carrier or excipient. In one aspect, such composition is a pharmaceutical composition, and, in certain embodiments, the carrier or excipient is a pharmaceutically acceptable carrier or pharmaceutically acceptable excipient.

The invention also includes polypeptide fragments of stress and/or shear resistant envelope protein polypeptides of the present invention. In one aspect, the polypeptide fragments have stress and/or shear resistant properties as described herein. Each of these fragments comprises at least about 20, 30, 50, 75, 100, 200, 300, 400, 500, 600, 620, 625, 650, 660, 655, 670, 671, 672, 673, 674, or 675 amino acids. Polypeptide fragments can also be made using the techniques described herein.

The present invention also includes at least one polypeptide consensus sequence derived from a comparison of two or more polypeptide sequences of the invention described herein. For example, the present invention includes at least one polypeptide consensus sequence derived from a comparison of two or more stress or shear resistant polypeptide sequences described herein. A polypeptide consensus sequence as used herein refers to a nonnaturally-occurring or recombinant or modified polypeptide that predominantly includes those amino acid residues that are common to all polypeptides of the present invention described herein (e.g., full-length polypeptides and fragments having stress or shear resistant properties described herein) and that includes, at one or more of those positions wherein there is no amino acid common to all subtypes, an amino acid that predominantly occurs at that position and in no event includes any amino acid residue that is not extant in that position in at least one polypeptide of the invention (e.g., stress or shear resistant polypeptide). A polypeptide consensus sequence may be resistant to the stress or shear forces of centrifugation under the conditions described herein.

Compositions comprising any retrovirus or retroviral polypeptide of the invention (or fragment thereof) or combination of any such retroviruses or polypeptides (or fragments thereof) are also an aspect of the invention.

Making Polypentides of the Invention

Recombinant methods for producing and isolating envelope protein polypeptides of the invention are described above. In addition to recombinant production, the polypeptides may be produced by direct peptide synthesis using solid-phase techniques (cf. Stewart et al. (1969) Solid-Phase Peptide Synthesis, W. H. Freeman Co., San Francisco; Merrifield J (1963) J. Am. Chem. Soc. 85:2149-2154) and other standard peptide synthesis techniques known to one of ordinary skill in the art. Peptide synthesis may be performed using manual techniques or by automation. Automated synthesis may be achieved, for example, using Applied Biosystems 43 1A Peptide Synthesizer (Perkin Elmer, Foster City, Calif.) in accordance with the instructions provided by the manufacturer. For example, subsequences may be chemically synthesized separately and combined using chemical methods to provide full-length envelope proteins.

Additional methods for producing the polypeptides of the invention are included. One such method comprises introducing into a population of cells any nucleic acid of the invention described herein, which is operatively linked to a regulatory sequence effective to produce the encoded polypeptide, culturing the cells in a culture medium to produce the polypeptide, and isolating the polypeptide from the cells or from the culture medium. An amount of nucleic acid sufficient to facilitate uptake by the cells (transfection) and/or expression of the polypeptide is utilized. The culture medium can be any described herein and in the Examples. The nucleic acid is introduced into such cells by any delivery method described herein, including, e.g., injection, gene gun, passive uptake, etc. The nucleic acid may be part of a vector, such as a recombinant expression vector, including a DNA plasmid vector, or any vector described herein. The nucleic acid or vector comprising a nucleic acid of the invention described herein may be prepared and formulated as described herein. Such a nucleic acid or expression vector may be introduced into a population of cells of a mammal in vivo, or selected cells of the mammal (e.g., tumor cells) may be removed from the mammal and the nucleic acid expression vector introduced ex vivo into the population of such cells in an amount sufficient such that uptake and expression of the encoded polypeptide results. Or, a nucleic acid or vector comprising a nucleic acid is produced using cultured cells in vitro. In one aspect, the method of producing a polypeptide comprises introducing into a population of cells a recombinant expression vector comprising any nucleic acid described herein in an amount and formula such that uptake of the vector and expression of the polypeptide will result; administering the expression vector into a mammal by any introduction/delivery format described herein; and isolating the pqlypeptide from the mammal or from a byproduct of the mammal.

Each of the nucleic acid and amino acid sequences corresponding to clones 4-4, 4-7, 2B-17, 2B-13, and 2B-8 and other nucleic acids and polypeptides of the invention can be made using standard techniques for synthesizing nucleotide and polypeptide sequences, including those described herein.

In another aspect, the invention provides for the use of any retrovirus envelope polypeptide or nucleic acid (or vector or cell comprising such nucleic acid) or composition thereof for the manufacture of a medicament, prophylactic, therapeutic, drug, or vaccine, including for any therapeutic or prophylactic application relating to treatment of a disease or disorder as described herein.

Using Polypeptides of the Invention

The retroviral envelopes polypeptides of the invention are useful in a variety of applications. In one aspect, the retroviral envelope protein polypeptides of the invention that exhibit resistance to the stress or shear forces of centrifugation are useful in making retroviruses for gene therapy, including in vivo human gene therapy. A stress or shear resistant retroviral envelope protein polypeptide of the invention can be incorporated into a retrovirus as an envelope protein to augment the resistance of the retrovirus to ultracentrifugation and thus improve the stability and manufacturing yield of the retrovirus.

Adjuvants

In another aspect, the stress and/or shear resistant envelope protein polypeptides of the present invention or fragments thereof are useful as adjuvants to stimulate or augment an immune response related to an antigen delivered by a retroviral vector (e.g., DNA vaccine) incorporating the stress and/or shear resistant envelope protein to a target tissue, cell, or organ. In one aspect, the invention provides therapeutic or prophylactic methods comprising administering one or more polypeptides of the invention described above as an adjuvant to a subject, including, e.g., mammal, including, e.g., a human, primate, mouse, pig, cow, goat, rabbit, rat, guinea pig, hamster, horse, sheep; or a non-mammalian vertebrate such as a bird (e.g., a chicken or duck) or a fish, or invertebrate.

Antibodies

In another aspect of the invention, a stress or shear resistant envelope protein, or subsequence or fragment thereof, of the invention is used to produce antibodies that have, e.g., diagnostic and therapeutic or prophylactic uses, e.g., related to the activity, distribution, and expression of retrovirus sequences.

Antibodies to envelope proteins of the invention can be generated by methods well known in the art. Such antibodies may include, but are not limited to, polyclonal, monoclonal, chimeric, humanized, single chain, Fab fragments and fragments produced by an Fab expression library. Antibodies, i.e., those that block receptor binding, are especially preferred for therapeutic or prophylactic use.

Envelope protein polypeptides for antibody induction do not require biological activity; however, the polypeptide or oligopeptide must be antigenic. Peptides used to induce specific antibodies may have an amino acid sequence consisting of at least 10 amino acids, preferably at least 15 or 20 amino acids. Short stretches of an envelope protein polypeptide may be fused with another protein, such as keyhole limpet hemocyanin, and antibody produced against the chimeric molecule.

Methods of producing polyclonal and monoclonal antibodies are known to those of ordinary skill in the art, and many antibodies are available. See, e.g., Coligan (1991) Current Protocols in Immunology Wiley/Greene, NY; and Harlow and Lane (1989) Antibodies: A Laboratory Manual Cold Spring Harbor Press, NY; Stites et al.(eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, N.Y.; and Kohler and Milstein (1975) Nature 256:495-497. Other suitable techniques for antibody preparation include selection of libraries of recombinant antibodies in phage or similar vectors. See Huse et al. (1989) Science 246:1275-1281; and Ward et al. (1989) Nature 341:544-546. Specific monoclonal and polyclonal antibodies and antisera (or antiserum) will usually bind with a K_{D} of at least about 0.1 μM, preferably at least about 0.01 μM or better, and most typically and preferably, 0.001 μM or better.

Detailed methods for preparation of chimeric (humanized) antibodies can be found in, e.g., U.S. Pat. No. 5,482,856. Additional details on humanization and other antibody production and engineering techniques can be found in Borrebaeck (ed.) (1995) Antibody Engineering, 2^{nd} Edition, Freeman and Company, NY (“Borrebaeck”); McCafferty et al. (1996) Antibody Engineering, A Practical Approach, IRL at Oxford Press, Oxford, England (“McCafferty”), and Paul (1995) Antibody Engineering Protocols, Humana Press, Towata, N.J. (hereinafter “Paul, Antibody Engineering Protocols”).

In one useful embodiment, this invention provides for fully humanized antibodies against the envelope proteins of the invention. Humanized antibodies are especially desirable in applications where the antibodies are used as therapeutics or prophylactic in vivo in human patients. Human antibodies consist of characteristically human immunoglobulin sequences. The human antibodies of this invention can be produced in using a wide variety of methods (see, e.g., Larrick et al., U.S. Pat. No. 5,001,065, and Borrebaeck, McCafferty, and Paul, Antibody Engineering Protocols, supra, for a review). In one embodiment, the human antibodies of the present invention are produced initially in trioma cells. Genes encoding the antibodies are then cloned and expressed in other cells, such as nonhuman mammalian cells. The general approach for producing human antibodies by trioma technology is described by Ostberg et al. (1983) Hybridoma 2:361-367, Ostberg, U.S. Pat. No. 4,634,664, and Engelman et al., U.S. Pat. No. 4,634,666. The antibody-producing cell lines obtained by this method are called triomas because they are descended from three cells; two human and one mouse. Triomas have been found to produce antibody more stably than ordinary hybridomas made from human cells.

SEQUENCE VARIATIONS

Silent Variations

It will be appreciated by those skilled in the art that due to the degeneracy of the genetic code, a multitude of nucleic acids sequences encoding envelope protein polypeptides of the invention may be produced, some of which may bear minimal sequence homology to the nucleic acid sequences explicitly disclosed herein.

TABLE 1
Codon Table

| Amino acids | | | Codon | |
|---|---|---|---|---|
| Alanine | Ala | A | GCA | GCC GCG GCU |
| Cysteine | Cys | C | UGC | UGU |
| Aspartic acid | Asp | D | GAC | GAU |
| Glutamic acid | Glu | E | GAA | GAG |
| Phenylalanine | Phe | F | UUC | UUU |
| Glycine | Gly | G | GGA | GGC GGG GGU |
| Histidine | His | H | CAC | CAU |
| Isoleucine | Ile | I | AUA | AUC AUU |
| Lysine | Lys | K | AAA | AAG |
| Leucine | Leu | L | UUA | UUG CUA CUC CUG CUU |
| Methionine | Met | M | AUG | |
| Asparagine | Asn | N | AAC | AAU |
| Proline | Pro | P | CCA | CCC CCG CCU |
| Glutamine | Gln | Q | CAA | CAG |
| Arginine | Arg | R | AGA | AGG CGA CGC CGG CGU |
| Serine | Ser | S | AGC | AGU UCA UCC UCG UCU |
| Threonine | Thr | T | ACA | ACC ACG ACU |
| Valine | Val | V | GUA | GUC GUG GUU |
| Tryptophan | Trp | W | UGG | |
| Tyrosine | Tyr | Y | UAC | UAU |

For instance, inspection of the codon table (Table 1) shows that codons AGA, AGG, CGA, CGC, CGG, and CGU all encode the amino acid arginine. Thus, at every position in the nucleic acids of the invention where an arginine is specified by a codon, the codon can be altered to any of the corresponding codons described above without altering the encoded polypeptide. It is understood that U in an RNA sequence corresponds to T in a DNA sequence.

Such “silent variations” are one species of “conservatively modified variations,” discussed below. One of ordinary skill in the art will recognize that each codon in a nucleic acid (except AUG and UGG, which is ordinarily the only codon for methionine and tryptophan, respectively) can be modified by standard techniques to encode a functionally identical polypeptide. Accordingly, each silent variation of a nucleic acid that encodes a polypeptide is implicit in any described sequence. The invention provides each and every possible variation of nucleic acid sequence encoding a polypeptide of the invention that could be made by selecting combinations based on possible codon choices. These combinations are made in accordance with the standard triplet genetic code (e.g., as set forth in Table 1) as applied to the nucleic acid sequence encoding an envelope protein polypeptide of the invention. All such variations of every nucleic acid herein are specifically provided and described by consideration of the sequence in combination with the genetic code.

Conservatively Modified Variations

Retrovirus envelope protein polypeptides of the present invention include one or more conservatively modified variations (or simply “conservative variations”) of the sequences disclosed herein as SEQ ID NOS:4-8. Such conservatively modified variations comprise substitutions, additions or deletions that alter, add or delete a single amino acid or a small percentage of amino acids (typically less than about 5%, more typically less than about 4%, 2%, or 1%) in any of SEQ ID NOS:4-8. One of ordinary skill in the art will recognize that an individual substitution, deletion, or addition that substitutes, deletes, or adds a single amino acid or a small percentage of amino acids (typically less than 5%, more typically less than 4%, 2% or 1%, or less) in a sequence typically constitute conservatively modified variations where such changes result in the deletion of an amino acid, addition of an amino acid, or substitution of an amino acid with a chemically similar amino acid.

For example, a conservatively modified variation (e.g., deletion) of the 672 amino acid polypeptide identified herein as SEQ ID NO:4 has a length of at least 638 amino acids, preferably at least 645 amino acids, more preferably at least 659 amino acids, and still more preferably at least 665 amino acids, corresponding to a deletion of less than about 5%, 4%, 2% or 1% of the polypeptide sequence, respectively.

Another example of a conservatively modified variation (e.g., a “conservatively substituted variation”) of the polypeptide identified herein as SEQ ID NO:4 contains “conservative substitutions,” according to the six substitution groups set forth in Table 2, in up to about 33 residues (i.e., less than about 5%) of the 672 amino acid polypeptide.

Conservative substitution tables providing functionally similar amino acids are well known those of ordinary skill in the art. Table 2 sets forth si× groups which contain amino acids that are “conservative substitutions” or “conservative variations” for one another.

TABLE 2
Conservative Substitution Groups

| | | | | |
|---|---|---|---|---|
| 1 | Alanine (A) | Serine (S) | Threonine (T) | |
| 2 | Aspartic acid (D) | Glutamic acid (E) | | |
| 3 | Asparagine (N) | Glutamine (Q) | | |
| 4 | Arginine (R) | Lysine (K) | | |
| 5 | Isoleucine (I) | Leucine (L) | Methionine (M) | Valine (V) |
| 6 | Phenylalanine (F) | Tyrosine (Y) | Tryptophan (W) | |

Additional groups of amino acids can also be formulated. For example, amino acids can be grouped by similar function or chemical structure or composition (e.g., acidic, basic, aliphatic, aromatic, sulfur-containing). For example, an aliphatic grouping may comprise: Glycine (G), Alanine, Valine, Leucine, Isoleucine. Other groups containing amino acids that are conservative substitutions for one another include: Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). See also Creighton (1984) Proteins, W. H. Freeman and Company, for additional groupings of amino acids.

The stress and/or shear resistant envelope protein sequences of the invention, including conservatively substituted sequences, can be present as part of larger polypeptide sequences such as occur upon the addition of one or more domains for purification of the protein (e.g., poly his segments, Fla.AG tag segments, etc.), e.g., where the additional functional domains have little or no effect on the activity of the envelope protein portion of the protein, or where the additional domains can be removed by post synthesis processing steps, such as by treatment with a protease.

As an example, if four conservative substitutions were localized in the subsequence corresponding to amino acids 151-175 of SEQ ID NO:4, examples of conservatively substituted variations of this subsequence,

VCPGP HRPRE AKSCG GPDSF YCASW include:

LCPGP HKPRE AKSCG GPESF YCTSW and

VCPGP HRPRD SKSCG GPDSY YCATW and the like, where the conservative substitutions are underlined.

A feature of the invention is an envelope protein polypeptide comprising at least 20 contiguous amino acids of any one of SEQ ID NOS:4-8.

In various embodiments, the polypeptide comprises at least about 100, 200, 300, 400, 500, 600, 615, 620, 650, 660, 665, or 670 contiguous amino acid residues of any one of SEQ ID NOS:4-8.

In other embodiments, the polypeptide is at least 620 amino acids, at least 638 amino acids, preferably at least 645 amino acids, more preferably at least 659 amino acids, and still more preferably at least 665 amino acids in length. In other embodiments of the invention, the polypeptide is preferably at least 670, 671, 672, 673, 674, or 675 amino acids in length.

The addition of one or more nucleic acids or sequences that do not alter the encoded activity of a nucleic acid molecule of the invention, such as the addition of a non-functional sequence, is a conservative variation of the basic nucleic acid molecule, and the addition of one or more amino acid residues that do not alter the activity of a polypeptide of the invention is a conservative variation of the basic polypeptide. Both such types of additions are features of the invention.

The envelope protein sequences of the invention, including conservatively substituted sequences, can be present as part of larger polypeptide sequences such as occur upon the addition of one or more domains for purification of the protein (e.g., poly his segments, Fla.AG tag segments, etc.), e.g., where the additional functional domains have little or no effect on the activity of the envelope protein portion of the protein, or where the additional domains can be removed by post synthesis processing steps such as by treatment with a protease.

One of ordinary skill in the art will appreciate that many conservative variations of the nucleic acid constructs which are disclosed yield a functionally identical construct. For example, as discussed above, owing to the degeneracy of the genetic code, “silent substitutions” (i.e., substitutions in a nucleic acid sequence which do not result in an alteration in an encoded polypeptide) are an implied feature of every nucleic acid sequence which encodes an amino acid. Similarly, “conservative amino acid substitutions,” in one or a few amino acids in an amino acid sequence are substituted with different amino acids with highly similar properties, are also readily identified as being highly similar to a disclosed construct. Such conservative variations of each disclosed sequence are a feature of the present invention.

PERCENT SEQUENCE IDENTITY—SEQUENCE SIMILARITY

The degree to which one nucleic acid is similar to another provides an indication of whether there is an evolutionary relationship between the two or more nucleic acids. In particular, where a high level of sequence identity is observed, it is inferred that the nucleic acids are derived from a common ancestor (i.e., that the nucleic acids are homologous). In addition, sequence similarity implies similar structural and functional properties for the two or more nucleic acids and the sequences they encode. Accordingly, in the context of the present invention, sequences which have a similar sequence to any given exemplar sequence are a feature of the present invention. In particular, sequences that have share percent sequence identities as defined below are a feature of the invention.

A variety of methods of determining sequence relationships can be used, including manual alignment and computer assisted sequence alignment and analysis. This later approach is a preferred approach in the present invention, due to the increased throughput afforded by computer-assisted methods. A variety of computer programs for performing sequence alignment are available, or can be produced by one of skill.

As noted above, the sequences of the nucleic acids and polypeptides (and fragments thereof) employed in the subject invention need not be identical, but can be substantially identical (or substantially similar), to the corresponding sequence of a retrovirus envelope polypeptide or nucleic acid molecule (or fragment thereof) of the invention or related molecule. For example, the polypeptides can be subject to various changes, such as one or more amino acid or nucleic acid insertions, deletions, and substitutions, either conservative or non-conservative, including where, e.g., such changes might provide for certain advantages in their use, e.g., in their therapeutic or prophylactic use or administration or diagnostic application. The nucleic acids can also be subject to various changes, such as one or more substitutions of one or more nucleic acids in one or more codons such that a particular codon encodes the same or a different amino acid, resulting in either a conservative or non-conservative substitution, or one or more deletions of one or more nucleic acids in the sequence. The nucleic acids can also be modified to include one or more codons that provide for optimum expression in an expression system (e.g., mammalian cell or mammalian expression system), while, if desired, said one or more codons still encode the same amino acid(s). Such nucleic acid changes might provide for certain advantages in their therapeutic or prophylactic use or administration, or diagnostic application. The nucleic acids and polypeptides can be modified in a number of ways so long as they comprise a sequence substantially identical (as defined below) to a sequence in a respective retrovirus envelope polypeptide or corresponding nucleic acid of the invention described herein.

Alignment and comparison of relatively short amino acid sequences (less than about 30 residues) is typically straightforward. Comparison of longer sequences can require more sophisticated methods to achieve optimal alignment of two sequences. Optimal alignment of sequences for aligning a comparison window can be conducted by the local homology algorithm of Smith and Waterman (1981) Adv Appl Math 2:482, by the homology alignment algorithm of Needleman and Wunsch (1970) J Mol Biol 48:443, by the search for similarity method of Pearson and Lipman (1988) Proc Nat'l Acad Sci USA 85:2444, by computerized implementations of these algorithms (GAP, BESTFIT, FASTA and TFASTA in the Wisconsin Genetics Software Package Release 7.0, Genetics Computer Group, 575 Science Dr., Madison, Wis.; and BLAST, see, e.g., Altschul et al. (1977) Nuc Acids Res 25:3389-3402 and Altschul et al. (1990) J Mol Biol 215:403-410), or by inspection, with the best alignment (i.e., resulting in the highest percentage of sequence similarity or sequence identity over the comparison window) generated by the various methods being selected.

The term “identical” or percent “identity,” in the context of two or more nucleic acid or polypeptide sequences, refers to two or more sequences or subsequences that are the same or have a specified percentage of amino acid residues or nucleotides that are the same, when compared and aligned for maximum correspondence, as measured using one of the following sequence comparison algorithms or by visual inspection.

The term “sequence identity” or “percent identity” (“% identity”) means that two polynucleotide or polypeptide sequences are identical (i.e., on a nucleotide-by-nucleotide basis or amino acid-by-amino acid basis, respectively) over a window of comparison. The term “percentage of sequence identity” (or “percent sequence identity” or simply “percent identity” or “% identity”) or “percentage of sequence similarity” (or “percent sequence similarity” or simply “percent similarity”) is calculated by comparing two optimally aligned polynucleotide or polypeptide sequences over the window of comparison, determining the number of positions at which the identical residues occur in both sequences to yield the number of matched positions, dividing the number of matched positions by the total number of positions in the window of comparison (i.e., the window size), and multiplying the result by 100 to yield the percentage of sequence identity (or percentage of sequence similarity). Thus, for example, with regard to polypeptide sequences, the term sequence identity means that two polypeptide sequences are identical (on an amino acid-by-amino acid basis) over a window of comparison, and a percentage of amino acid residue sequence identity (or percentage of amino acid residue sequence similarity), can be calculated. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Maximum correspondence can be determined by using one of the sequence algorithms described herein (or other algorithms available to those of ordinary skill in the art) or by visual inspection.

The phrase “substantially identical” or “substantial identity” in the context of two nucleic acids or polypeptides, refers to two or more sequences or subsequences that have at least about 50%, 60%, 70%, 75%, preferably 80%, or 85%, 86%, 87%, 88%, or 89%, more preferably 90%, 91%, 92%, 93%, 94%, 95%, 95.5%, 96%, 96.5%, 97%, 97.5%, 98%, 98.5%, 99%, 99.5%, or more nucleotide or amino acid residue % identity, respectively, when compared and aligned for maximum correspondence, as measured using one of the following sequence comparison algorithms or by visual inspection. In certain embodiments, the substantial identity exists over a region of amino acid sequences that is at least about 50 residues in length, preferably over a region of at least about 100 residues in length, and more preferably the sequences are substantially identical over at least about 150, 200, 250, 300, 400, 500, 600, 650, 655, 660, or 670 amino acid residues. In certain aspects, substantial identity exists over a region of nucleic acid sequences of at least about 500 residues, preferably over a region of at least about 600 residues in length, and more preferably the sequences are substantially identical over at least about 700, 800, 900, 1000, 1250, or 1500 nucleic acid residues. In some aspects, the amino acid or nucleic acid sequences are substantially identical over the entire length of the corresponding coding region.

As applied to polypeptides and peptides, the term “substantial identity” typically means that two polypeptide or peptide sequences, when optimally aligned, such as by the programs GAP or BESTFIT using default gap weights (described in detail below) or by visual inspection, share at least about 60% or 70%, often at least 75%, preferably at least about 80% or 85%, 86%, 87%, 88%, or 89%, more preferably at least about 90%, 91%, 92%, 93%, 94%, 95%, 95.5%, 96%, 96.5% 97%, 97.5%, 98%, 98.5%, 99%, or 99.5% or more percent amino acid residue sequence identity or sequence similarity. Similarly, as applied in the context of two nucleic acids, the term substantial identity or substantial similarity means that the two nucleic acid sequences, when optimally aligned, such as by the programs BLAST, GAP or BESTFIT using default gap weights (described in detail below) or by visual inspection, share at least about 60 percent, 70 percent, or 80 or 85 percent sequence identity ′or sequence similarity, preferably at least about 90 percent nucleotide sequence identity or sequence similarity, more preferably at least about 95 percent sequence identity or sequence similarity, or more, including, e.g., at least about 90, 91, 92, 93, 94, 95, 95.5, 96, 96.5, 97, 97.5, 98, 98.5, 99, 99.5, or more percent nucleotide sequence identity or sequence similarity.

In one aspect, the present invention provides nucleic acids encoding amino acid molecules (or fragments thereof) having at least about 50%, 60%, 70%, 75%, 80%, 85%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5% or more percent sequence identity or sequence similarity with the nucleic acid of any of SEQ ID NOS:1-3 and/or SEQ ID NOS:9-10 or a fragment thereof. Some such nucleic acids may have one or more stress or shear resistant properties and/or uses as described herein.

In yet another aspect, the present invention provides retrovirus envelope protein polypeptides that are substantially identical or substantially similar over at least about 400, 500, 550, 600, 625, 630, 640, 650, 655, 660, 665, 670, 671, 672, 673, 674, 675 or more contiguous amino acids of at least one of SEQ ID NOS:4-8. Some such polypeptides may have one or more properties and/or uses as described herein. A feature of the invention is a retrovirus envelope polypeptide comprising at least about 500, 550, 600, 625, 630, 640, 650, 655, 660, 665, 670, 671, 672, 673, 674, or 675 contiguous amino acids of any one of SEQ ID NOS:4-8.

Alternatively, parameters are set such that one or more sequences of the invention are identified by alignment to a query sequence selected from among SEQ ID NOS:4-8, while sequences corresponding to unrelated polypeptides, e.g., those encoded by known nucleic acid sequences represented by, e.g., GenBank accession numbers or other public databases (e.g., known retrovirus polypeptide sequences) are not identified.

Preferably, residue positions that are not identical differ by conservative amino acid substitutions. Conservative amino acid substitution refers to the interchange-ability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

Alignment and comparison of relatively short amino acid sequences (less than about 30 residues) is typically straightforward. Comparison of longer sequences can require more sophisticated methods to achieve optimal alignment of two sequences. Optimal alignment of sequences for aligning a comparison window can be conducted by the local homology algorithm of Smith and Waterman (1981) Adv Annl Math 2:482, by the homology alignment algorithm of Needleman and Wunsch (1970) J Mol Biol 48:443, by the search for similarity method of Pearson and Lipman (1988) Proc Nat'l Acad Sci USA 85:2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, with the best alignment (i.e., resulting in the highest percentage of sequence similarity over the comparison window) generated by the various methods being selected.

A preferred example of an algorithm that is suitable for determining percent sequence identity (percent identity) and sequence similarity is the FASTA algorithm, which is described in Pearson, W. R. & Lipman, D. J. (1988) Proc Nat'l Acad Sci USA 85:2444. See also, W. R. Pearson (1996) Methods Enzymology 266:227-258. Preferred parameters used in a FASTA alignment of DNA sequences to calculate percent identity are optimized, BL50 Matrix 15: −5, k-tuple=2; joining penalty=40, optimization=28; gap penalty −12, gap length penalty=−2; and width=16.

Other preferred examples of algorithm that are suitable for determining percent sequence identity and sequence similarity are the BLAST and BLAST 2.0 algorithms, which are described in Altschul et al. (1997) Nuc Acids Res 25:3389-3402 and Altschul et al. (1990) J Mol Biol 215:403-410, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http: Hlwww.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls o,ff-by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program (e.g., BLASTP 2.0.14; Jun-29-2000) uses as defaults a wordlength of 3, and expectation (E) of 10, and the BLOSUM62 scoring matrix (see, Henikoff & Henikoff (1989) Proc Nat'l Acad Sci USA 89:10915) uses alignments (B) of 50, expectation (E) of 10, M=5, N=−4, and a comparison of both strands. Again, as with other suitable algorithms, the stringency of comparison can be increased until the program identifies only sequences that are more closely related to those in the sequence listings herein (i.e., SEQ ID NOS:1-3 and/or SEQ ID NOS:9-10, or, alternatively, SEQ ID NOS:4-8, rather than sequences that are more closely related to other similar sequences such as, e.g., those nucleic acid sequences represented by GenBank accession numbers set forth herein, and or other similar molecules found in, e.g., GenBank. In other words, the stringency of comparison of the algorithms can be increased so that all known prior art (e.g., those represented by GenBank accession numbers shown herein, or other similar molecules found in, e.g., GenBank) is excluded.

The BLAST algorithm also performs a statistical analysis of the similarity or identity between two sequences (see, e.g., Karlin & Altschul (1993) Proc Nat'l Acad Sci USA 90:5873-5787). One measure of similarity provided by this algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another example of a useful algorithm is PILEUP. PILEUP creates a multiple sequence alignment from a group of related sequences using progressive, pairwise alignments to show relationship and percent sequence identity or percent sequence similarity. It also plots a tree or dendogram showing the clustering relationships used to create the alignment. PILEUP uses a simplification of the progressive alignment method of Feng & Doolittle (1987) J Mol Evol 35:351-360. The method used is similar to the method described by Higgins & Sharp (1989) CABIOS 5:151-153. The program can align up to 300 sequences, each of a maximum length of 5,000 nucleotides or amino acids. The multiple aligrnment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity (or percent sequence similarity) relationship using the following parameters: default gap weight (3.00), default gap length weight (0.10), and weighted end gaps. PILEUP can be obtained from the GCG sequence analysis software package, e.g., version 7.0 (Devereaux et al. (1984) Nuc Acids Res 12:387-395).

Another preferred example of an algorithm that is suitable for multiple DNA and amino acid sequence alignments is the CLUSTALW program (Thompson, J. D. et al. (1994) Nuc Acids Res 22:4673-4680). CLUSTALW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix (Henikoff and Henikoff (1992) Proc Nat'l Acad Sci USA 89:10915-10919). Another example of an algorithm suitable for multiple DNA and amino acid sequence alignments is the Jotun Hein method, Hein (1990), from within the MegaLine™ DNASTAR package (MegaLine™ Version 4.03, manufactured by DNASTAR, Inc.) used according to the manufacturer's instructions and default values specified in the program.

It will be understood by one of ordinary skill in the art, that the above discussion of search and alignment algorithms also applies to identification and evaluation of polynucleotide sequences, with the substitution of query sequences comprising nucleotide sequences, and where appropriate, selection of nucleic acid databases.

RETROVIRUSES OF THE INVENTION

In another aspect, the present invention provides retrovirus comprising a novel stress and/or shear retrovirus envelope protein polypeptide as an envelope protein, fragments of such envelope protein polypeptides, related fusion polypeptides or proteins, or functional equivalents thereof, as outlined in greater detail above and hereinafter. Retroviruses of the invention include those of the family Retroviridae, including those of the subfamilies, Oncovirinae (e.g., murine leukemia virus), Spumavirinae, and Lentivirinae (e.g., lentivirus), and respective genera and subgenera of these subfamilies. For a discussion of retroviruses, see White, D. O. and Fenner, F. J., MEDICAL VIROLOGY (3d ed. 1986), which is incorporated herein by reference in its entirety for all purposes. Included are exogenous and endogenous retroviruses. The retroviruses may be ecotropic (i.e., only multiply in the host species from which they originate), amphotropic (can multiply in particular native and foreign hosts), or xenotropic (cannot multiply in host species in whose genome they are carried as provirus, but can infect other species that are not necessarily related). Id.

In some aspects, retroviruses of the invention are useful in therapeutic or prophylactic compositions, as described in detail below, and for use in methods of the invention, including gene therapy and ex vivo, in vivo, and in vitro applications described supra and below. In another aspect, the stress and/or shear resistant retroviruses of the invention are useful in retroviral applications in which improved manufacturability and improved manufacturing yield compared to known retroviruses an retroviral vectors is desired, including in gene therapy methods utilizing retroviruses.

In another aspect, retroviruses of the invention that incorporate stress and/or shear resistant envelope protein polypeptides of the present invention or fragments thereof are useful as adjuvants to stimulate or augment an immune response to related to an antigen delivered by a retroviral vector (e.g., DNA vaccine) incorporating the stress and/or shear resistant envelope protein to a target tissue, cell, or organ. In another aspect, a retrovirus of the invention that includes a stress and/or shear resistant envelope protein polypeptide or a fragment thereof as an envelope protein is useful to produce antibodies which have, e.g., diagnostic and therapeutic or prophylactic uses, e.g., related to the activity, distribution, and expression of retrovirus sequences.

THERAPEUTIC AND PROPHYLACTIC APPLICATIONS

The invention includes compositions comprising polynucleotides and/or polypeptides of the invention in combination with a suitable carrier or excipient. In one aspect, therapeutic and prophylactic compositions are provided. Such compositions comprise, respectively, a therapeutically or prophylactically effective amount of at least one polynucleotide and/or polypeptide of the invention, and a carrier or excipient. The invention also provides pharmaceutical compositions that comprise therapeutically or prophylactically effective amount of at least one polynucleotide and/or polypeptide of the invention, and a pharmaceutically acceptable carrier or pharmaceutically acceptable excipient. The, carrier or excipient includes, but is not limited to, saline, buffered saline, dextrose, water, glycerol, ethanol, and combinations thereof. The formulation should suit the mode of administration. Methods of administering nucleic acids, polypeptides and proteins are well known in the art, and further discussed below.

Therapeutic and prophylactic compositions comprising one or more retroviruses comprising a stress and/or shear resistant envelope protein(s) of the invention, or retrovirus envelope protein(s) of the invention are tested in appropriate in vitro and in vivo animal models of disease, to confirm efficacy, tissue metabolism, and to estimate dosages, according to methods well known in the art. In particular, dosages can be determined by activity comparison of the retroviruses comprising the envelope proteins of the invention, e.g., to existing retroviral vectors, i.e., in a relevant assay. Typically, stress (and/or shear) resistant retroviruses having the envelope proteins of the invention and further comprising a therapeutic or prophylactic gene construct are administered, e.g., for gene therapy.

Genetic Vectors

Gene therapy and genetic vaccine vectors are useful for treating and/or preventing various diseases and other conditions. The following discussion focuses on the on the use of vectors because gene therapy and genetic vaccine method typically employ vectors, but persons of skill in the art appreciate that the nucleic acids of the invention can, depending on the particular application, be employed in the absence of vector sequences. Accordingly, references in the following discussion to vectors should be understood as also relating to nucleic acids of the invention that lack vector sequences.

Vectors can be delivered to a subject to induce an immune response or other therapeutic or prophylactic response. Suitable subjects include, but are not limited to, a mammal, including, e.g., a human, primate, monkey, orangutan, baboon, mouse, pig, cow, cat, goat, rabbit, rat, guinea pig, hamster, horse, sheep; or a non-mammalian vertebrate such as a bird (e.g., a chicken or duck) or a fish, or invertebrate.

Vectors can be delivered in vivo by administration to an individual patient, typically by local (direct) administration or by systemic administration (e.g., intravenous, intraperitoneal, intramuscular, subdermal, intracranial, anal, vaginal, oral, buccal route or they can be inhaled) or they can be administered by topical application. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

In local (direct) administration formats, the nucleic acid or vector is typically administered or transferred directly to the cells to be treated or to the tissue site of interest (e.g., tumor cells, tumor tissue sample, organ cells, blood cells, cells of the skin, lung, heart, muscle, brain, mucosae, liver, intestine, spleen, stomach, lymphatic system, cervi×, vagina, prostate, mouth, tongue, etc.) by any of a variety of formats, including topical administration, injection (e.g., by using a needle or syringe), or vaccine or gene gun delivery, pushing into a tissue, organ, or skin site. For standard gene gun administration, the vector or nucleic acid of interest is precipitated onto the surface of microscopic metal beads. The microprojectiles are accelerated with a shock wave or expanding helium gas, and penetrate tissues to a depth of several cell layers. For example, the Accel™ Gene Delivery Device manufactured by Agacetus, Inc. Middleton Wis. is suitable for use in this embodiment. The nucleic acid or vector can be delivered, for example, intramuscularly, intradermally, subdermally, subcutaneously, orally, intraperitoneally, intrathecally, intravenously, or placed within a cavity of the body (including, e.g., during surgery), or by inhalation or vaginal or rectal administration.

In in vivo indirect contact/administration formats, the nucleic acid or vector is typically administered or transferred indirectly to the cells to be treated or to the tissue site of interest, including those described above (such as, e.g., skin cells, organ systems, lymphatic system, or blood cell system, etc.), by contacting or administering the nucleic acid or vector of the invention directly to one or more cells or population of cells from which treatment can be facilitated. For example, tumor cells within the body of the subject can be treated by contacting cells of the blood or lymphatic system, skin, or an organ with a sufficient amount of the polypeptide such that delivery of the nucleic acid or vector to the site of interest (e.g., tissue, organ, or cells of interest or blood or lymphatic system within the body) occurs and effective prophylactic or therapeutic treatment results. Such contact, administration, or transfer is typically made by using one or more of the routes or modes of administration described above.

A large number of delivery methods are well known to those of skill in the art. Such methods include, for example liposome-based gene delivery (Debs and Zhu (1993) WO 93/24640; Mannino and Gould-Fogerite (1988) BioTechniques 6(7):682-691; Rose U.S. Pat No. 5,279,833; Brigham (1991) WO 91/06309; and Felgner et al. (1987) Proc. Nat'l Acad. Sci. USA 84:7413-7414), as well as use of viral vectors (e.g., adenoviral (see, e.g., Berns et al. (I 995) Ann. NY Acad. Sci. 772:95-104; Ali et al. (1994) Gene Ther. 1:367-384; and Haddada et al. (1995) Curr. Top. Microbiol. Immunol. 199 (Pt 3):297-306 for review), papillomaviral, retroviral (see, e.g., Buchscher et al. (1992) J. Virol. 66(5)2731-2739; Johann et al. (1992) J. Virol. 66 (5):1635-1640 (1992); Sommerfelt et al. (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed.) Raven Press, Ltd., New York and the references therein, and Yu et al., Gene Therapy (1994) supra), and adeno-associated viral vectors (see West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 and Samulski (supra) for an overview of AAV vectors; see also, Lebkowski, U.S. Pat. No. 5,173,414; Tratschin et al. (1985) Mol. Cell. Biol. 5(11):3251-3260; Tratschin et al. (1984) Mol. Cell. Biol. 4:2072-2081; Hermonat and Muzyczka (1984) Proc. Nat'l Acad. Sci. USA 81:6466-6470; McLaughlin et al. (1988) and Samulski et al. (1989) J. Virol. 63:03822-3828), and the like.

“Naked” DNA and/or RNA that comprises a genetic vaccine can also be introduced directly into a tissue, such as muscle, by injection using a needle or other similar device. See, e.g., U.S. Pat. No. 5,580,859. Other methods such as “biolistic” or particle-mediated transformation (see, e.g., Sanford et al., U.S. Pat. No. 4,945,050; U.S. Pat. No. 5,036,006) are also suitable for introduction of genetic vaccines into cells of a mammal according to the invention. These methods are useful not only for in vivo introduction of DNA into a subject, such as a mammal, but also for ex vivo modification of cells for reintroduction into a mammal. DNA is conveniently introduced directly into the cells of a mammal or other subject using, e.g., injection, such as via a needle, or a “gene gun.” As for other methods of delivering genetic vaccines, if necessary, vaccine administration is repeated in order to maintain the desired level of immunomodulation, such as the level or response of T cell activation or T cell proliferation, or antibody titer level. Alternatively, nucleotides can be impressed into the skin of the subject.

Gene therapy and genetic vaccine vectors (e.g., DNA, plasmids, expression vectors, adenoviruses, liposomes, papillomaviruses, retroviruses, etc.) comprising at least one nucleic acid sequence of the invention can be administered directly to the subject (usually a mammal) for transduction of cells in vivo. The vectors can be formulated as phanpaceutical compositions for administration in any suitable manner, including parenteral (e.g., subcutaneous, intramuscular, intradermal, or intravenous), topical, oral, rectal, vaginal, intrathecal, buccal (e.g., sublingual), or local administration, such as by aerosol or transdermally, for immunotherapeutic or other prophylactic and/or therapeutic treatment. Pretreatment of skin, for example, by use of hair-removing agents, may be useful in transdermal delivery. Suitable methods of administering such packaged nucleic acids are available and well known to those of skill in the art, and, although more than one route can be used to administer a particular composition, a particular route can often provide a more immediate and more effective reaction than another route.

Ex vivo applications

Ex vivo methods for introducing a therapeutic or prophylactic gene into a cell or organism frequently involve transducing the cell ex vivo with a therapeutic or prophylactic nucleic acid or gene construct of this invention, and introducing the cell into the organism. Target cells include CD4+ cells such as CD4+ T cells or macrophage isolated or cultured from a patient, stem cells, or the like. See, e.g., Freshney et al., supra and the references cited therein for a discussion of how to isolate and culture cells from patients. Alternatively, the cells can be those stored in a cell bank (e.g., a blood bank). In one class of embodiments, the packageable nucleic acid encodes an anti-viral therapeutic or prophylactic agent (e.g., suicide gene, trans-dominant gene, anti-viral ribozyme, anti-sense gene, or decoy gene) that inhibits the growth or replication of a cell infected with a virus (e.g., HIV), or a virus, under the control of an activated or constitutive promoter. The cell transformation vector inhibits viral replication in any of those cells already infected with the subject virus, in addition to conferring a protective effect to cells that are not infected with the virus. Thus, the present invention provides a method of protecting cells in vitro, ex vivo or in vivo, even when the cells are already infected with the virus against which protection is sought. Alternatively, the packageable nucleic acid encodes a therapeutic or prophylactic gene construct directed against a non-viral infectious agent. In other embodiments the packageable gene construct is selected to provide anti-oncogenic, or other anti-cancer, e.g., anti-metastatic, effects. In yet other embodiments, the therapeutic or prophylactic gene construct provides remediation or prophylaxis for a congenital or inborn error, e.g., of metabolism.

In some embodiments, stem cells (which are typically not CD4+) are used in ex vivo procedures for cell transformation and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α: are known (see Inaba et al. (1992) J. Exp. Med. 176, 1693-1702, and Szabolcs et al. (1995) J. Immunol._{—}154:5851-5861). Methods of pseudotyping retrovirus vectors so that they can transform stem cells are described above. An affinity column isolation procedure can be used to isolate cells which bind to CD34, or to antibodies bound to CD34. See Ho et al. (1995) Stem Cells 13 (suppl. 3):100-105. See also Brenner (1993) Journal of Hematotherapv 2:7-17. In another embodiment, hematopoietic stem cells are isolated from fetal cord blood. Yu et al. (1995) Proc. Nat'l Acad. Sci. USA 92:699-703 describe a preferred method of transducing CD34+ cells from human fetal cord a blood using retroviral vectors. Rather than using stem cells, T cells can also be transduced in ex vivo procedures. Several techniques are known for isolating T cells. In one method, Ficoll-Hypaque density gradient centrifugation is used to separate PBMC from red blood cells and neutrophils according to established procedures. Cells are washed with modified AIM-V (which consists or AIM-V (GIBCO) with 2 milliMolar (mM) glutamine, 10 micrograms/milliliter (μg/ml) gentamicin sulfate, 50 μg/ml streptomycin) supplemented with 1% fetal bovine serum (FBS). Enrichment for T cells is performed by negative or positive selection with appropriate monoclonal antibodies coupled to columns or magnetic beads according to standard techniques. An aliquot of cells is analyzed for desired cell surface phenotype (e.g., CD4, CD8, CD3, CD14, etc.).

In general, the expression of surface markers facilitates identification and purification of T cells. Methods of identification and isolation of T cells include FACS, column chromatography, panning with magnetic beads, western blots, radiography, electrophoresis, capillary electrophoresis, high performance liquid chromatography (HPLC), thin layer chromatography (TLC), hyperdiffusion chromatography, and the like, and various immunological methods such as fluid or gel precipitin reactions, immunodiffusion (single or double), immunoelectrophoresis, radioimmunoassays (RIAs), enzyme-linked immunoabsorbent assays (ELISAs), immunofluorescent assays, and the like. For a review of immunological and immunoassay procedures in general, see Stites and Terr (eds.)1991 Basic and Clinical Immunology (7th ed.) and Paul supra. For a discussion of how to make antibodies to selected antigens see, e.g., Coligan (1991) Current Protocols in Immunology Wiley/Greene, NY; and Harlow and Lane (1989) Antibodies: A Laboratory Manual Cold Spring Harbor Press, NY; Stites et al. (eds.) Basic and Clinical Immunology (4th Ads.).

In addition to the ex vivo uses described above, the packaging cell lines of the invention and the packageable nucleic acids of the invention are useful generally in cloning methods. Packageable nucleic acids are packaged in an retrovirus particle and used to transform an infectible cell (e.g., 293T cells) in vitro or in vivo. This provides one of ordinary skill in the art with a technique and vectors for transforming cells with a nucleic acid of choice, e.g., in drug discovery assays, or as a tool in the study of gene regulation, or as a general cloning vector.

In Vivo and Ex Vivo Transformation

Non-primate, e.g., murine, retroviral particles containing therapeutic or prophylactic nucleic acids can be administered directly to an organism for transduction of cells in vivo or ex vivo. In one aspect, the invention provides methods comprising administering one or more nucleotides of the invention described above to a subject; or mammal, including, e.g., a human, primate, mouse, pig, cow, goat, rabbit, rat, guinea pig, hamster, horse, sheep; or a non-mammalian vertebrate such as a bird (e.g., a chicken or duck) or a fish, or invertebrate.

Administration is by any of the routes normally used for introducing a molecule into ultimate contact with blood or tissue cells. The polypeptides and polynucleotides of the invention, and vectors, cells, and compositions comprising such molecules, are administered in any suitable manner, including, in some aspects, with a pharmaceutically acceptable carrier. Suitable methods of administering such molecules, in the context of the present invention, to a subject are available, and, although more than one route can be used to administer a particular composition, a particular route can often provide a more immediate and more effective reaction than another route. Preferred routes are readily ascertained by those of skill in the art.

Compositions and Formulations

Packageable nucleic acids packaged, e.g., in a stress resistant envelope protein are used to treat and prevent a variety of diseases, including virally mediated diseases, cancer, and other genetic disorders, in animals and human patients. The packaged nucleic acids are administered in any suitable manner, preferably with pharmaceutically acceptable carriers. Suitable methods of administering such packaged nucleic acids in the context of the present invention to a patient are available, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention.

Compositions comprising cells expressing at least one full length form of a polypeptide of the invention or a fragment thereof are also a feature of the invention. Such cells are readily prepared as described herein by transfection with DNA plasmid vector encoding at least one of the polypeptide of the invention. Compositions of such cells may comprise a pharmaceutically composition comprising a pharmaceutically acceptable carrier or excipient.

Pharmaceutical compositions of the invention can, but need not, include a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention. A variety of aqueous carriers can be used, e.g., buffered saline, such as PBS, and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of gene therapy or genetic vaccine vector in these formulations can vary widely, and will be selected primarily based on fluid volumes, viscosities, body weight and the like in accordance with the particular mode of administration selected and the patient's needs.

Compositions comprising polypeptides and polynucleotides, and vectors, cells, and other formulations comprising these and other components of the invention, can be administered by a number of routes including, but not limited to oral, intranasal, intravenous, intraperitoneal, intramuscular, transdermal, subcutaneous, intradermal, topical, sublingual, vaginal, or rectal means. Polypeptide and nucleic acid compositions can also be administered via liposomes. Such administration routes and appropriate formulations are generally known to those of skill in the art.

The polypeptide or polynucleotide of the invention or fragment thereof, or vector comprising a nucleic acid of the invention, alone or in combination with other suitable components, can also be made into aerosol formulations (e.g., they can be “nebulized”) to be administered via inhalation. Aerosol formulations can be placed into pressurized acceptable propellants, such as dichlorodifluoromethane, propane, nitrogen, and the like.

Formulations suitable for oral administration can consist of (a) liquid solutions, such as an effective amount of the packaged nucleic acid suspended in diluents, such as water, saline or PEG400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, tragacanth, microcrystalline cellulose, acacia, gelatin, colloidal silicon dioxide, croscarmellose sodium, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art. It is recognized that the gene therapy vectors and genetic vaccines, when administered orally, must be protected from digestion. This is typically accomplished either by complexing the vector with a composition to render it resistant to acidic and enzymatic hydrolysis or by packaging the vector in an appropriately resistant carrier such as a liposome. Means of protecting vectors from digestion are well known in the art. The pharmaceutical compositions can be encapsulated, e.g., in liposomes, or in a formulation that provides for slow release of the active ingredient. Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the packaged nucleic acid with a base, including, for example, liquid triglycerides, polyethylene glycols, and paraffin hydrocarbons.

Retrovirus particles incorporating therapeutic or prophylactic gene constructs can be administered by a number of routes including, but not limited to oral, intravenous, intraperitoneal, intramuscular, transdermal, subcutaneous, topical, sublingual, or rectal means. Such administration routes and appropriate formulations are generally knqwn to those of ordinary skill in the art.

The packaged nucleic acids, alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations of packaged nucleic acid can be presented in unit-dose or multi-dose sealed containers, such as ampules and vials.

Injection solutions and suspensions can be prepared from sterile powders, granules, and tablets of the kind previously described. Cells transduced by the packaged nucleic acid can also be administered to a subject intravenously, intramuscularly, or parenterally, or via another common route of administration.

Cells transduced as described above in the context of ex vivo or in vivo therapy can also be administered intravenously or parenterally as described above. It will be appreciated that the delivery of cells to a subject, e.g., human (patient), mammal, or other animal, is routine, e.g., methods of delivery of cells to the blood of the subject via intravenous, intramuscular, or intraperitoneal administration or other common route are known to those or ordinary skill in the art.

The dose administered to a patient, in the context of the present invention is sufficient to effect a beneficial therapeutic or prophylactic response in the patient over time. The dose will be determined by the efficacy of the particular therapeutic or prophylactic gene construct, retroviral vector, or formulation, and the titer or infectivity of the retroviruses employed and the condition of the patient, as well as the body weight or surface area of the patient to be treated. The size of the dose also will be determined by the existence, nature, and extent of any adverse side-effects that accompany the administration of a particular vector, formulation, transduced cell type or the like in a particular patient. Dosages to be used for therapeutic or prophylactic treatment of a particular disease or disorder can be determined by one of skill by comparison to those dosages used for existing therapeutic or prophylactic treatment protocols for the same disease or disorder.

In determining the effective amount of the vector, retrovirus, polypeptide, cell type, or formulation to be administered in the treatment or prophylaxis of inborn errors of metabolism, cancers, or infections, the physician evaluates circulating plasma levels, vector/retrovirus/polypeptide/cell/formulation toxicities, progression of the disease, and the production of antibodies directed against the vector or other aspect of the therapeutic or prophylactic composition.

The dose administered, e.g., to a 70 kilogram patient, will be in the range equivalent to dosages of currently-used retroviral gene therapies, and doses are calculated to yield an equivalent amount of therapeutic or prophylactic nucleic acid or expressed protein. In addition to remediating hereditary disorders, the vectors of this invention can be used to supplement treatment of cancers and virally-mediated conditions by any known conventional therapy, including cytotoxic agents, nucleotide analogues (e.g., when used for treatment of HIV infection), biologic response modifiers, and the like.

In one aspect, for example, in determining the effective amount of the vector to be administered in the treatment or prophylaxis of an infection or other condition, wherein the vector comprises any nucleic acid sequence of the invention described herein or encodes any polypeptide of the invention described herein, the physician evaluates vector toxicities, progression of the disease, and the production of anti-vector antibodies, if any. In one aspect, the dose equivalent of a naked nucleic acid from a vector for a typical 70 kilogram patient can range from about 10 nanogram (ng) to about 1 g, about 100 ng to about 100 mg, about 1 μg to about 10 mg, about 10 μg to about 1 mg, or from about 30-400 μg. Doses of vectors used to deliver the nucleic acid are calculated to yield an equivalent amount of therapeutic nucleic acid. Administration can be accomplished via single or divided doses.

In therapeutic applications, compositions are administered to a patient suffering from a disease in an amount sufficient to cure or at least partially arrest or ameliorate the disease or at least one of its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of protein to effectively treat the subject.

In prophylactic applications, compositions are administered to a human or other mammal to induce an immune or other prophylactic response that can help protect against the establishment of a disease, e.g., an infectious disease, cancer, autoimmune disorder, or other condition. For administration, the retroviral vectors and transduced cells of the present invention can be administered at a rate determined by the LD-50 of the therapeutic or prophylactic gene construct, vector, or transduced cell type, and the side-effects of the therapeutic or prophylactic compositions, vector or cell type at various concentrations, as applied to the mass and overall health of the patient. Administration can be accomplished via single or divided doses.

The toxicity and therapeutic efficacy of the retrovial vectors of the invention are determined using standard pharmaceutical procedures in cell cultures or experimental animals. One can determine the LD₅₀ (the dose lethal to 50% of the population) and the ED₅₀ (the dose therapeutically effective in 50% of the population) using procedures presented herein and those otherwise known to those of skill in the art. Nucleic acids, polypeptides, proteins, fusion proteins, transduced cells and other formulations of the present invention can be administered at a rate determined, e.g., by the LD₅₀ of the formulation, and the side-effects thereof at various concentrations, as applied to the mass and overall health of the patient. Again, administration can be accomplished via single or divided doses.

A typical pharmaceutical composition for intravenous administration would be about 0.1 microgram to 10 mg of the component of interest (e.g., vector) per patient per day. Dosages from 0.1 microgram up to about 100 mg per patient per day may be used, particularly when the component is administered to a secluded site and not into the blood stream, such as into a body cavity or into a lumen of an organ. Substantially higher dosages are possible in topical administration. Actual methods for preparing parenterally administrable compositions will be known or apparent to those skilled in the art and are described in more detail in such publications as Remington's Pharnaceutical Science, 15th ed., Mack Publishing Co., Easton, Pa. (1980).

For introduction of recombinant retrovirus infected cells into a patient, blood samples are obtained prior to infusion, and saved for analysis. Between 1×10⁶ and 1×10¹² transduced cells are infused intravenously over 60-200 minutes. Vital signs and oxygen saturation by pulse oximetry are closely monitored. Blood samples are obtained 5 minutes and 1 hour following infusion and saved for subsequent analysis. Leukopheresis, transduction and reinfusion are optionally repeated every 2 to 3 months for a total of 4 to 6 treatments in a one year period. After the first treatment, infusions can be performed on a outpatient basis at the discretion of the clinician. If the reinfusion is given as an outpatient, the participant is monitored for at least 4, and preferably 8 hours following the therapy. Transduced cells are prepared for reinfusion according to established methods. See Abrahamsen et al. (1991) J. Clin. Apheresis 6:48-53; Carter et al. (1988) J. Clin. Arpheresis 4:113-117; Aebersold et al. (1988) J. Immunol. Methods 112:1-7; Muul et al. (1987) J. Immunol. Methods 101: 171-181 and Carter et al. (1987) Transfusion 27:362-365. After a period of about 2-4 weeks in culture, the cells should number between 1×10⁶ and 1×10¹². In this regard, the growth characteristics of cells vary from patient to patient and from cell type to cell type. About 72 hours prior to reinfusion of the transduced cells, an aliquot is taken for analysis of phenotype, and percentage of cells expressing the therapeutic or prophylactic agent.

If a patient undergoing infusion of a vector or transduced cell or protein formulation develops fevers, chills, or muscle aches, he/she receives the appropriate dose of aspirin, ibuprofen, acetaminophen or other pain/fever controlling drug. Patients who experience reactions to the infusion such as fever, muscle aches, and chills are premedicated 30 minutes prior to the future infusions with either aspirin, acetaminophen, or, e.g., diphenhydramine. Meperidine is used for more severe chills and muscle aches that do not quickly respond to antipyretics and antihistamines. Cell infusion is slowed or discontinued depending upon the severity of the reaction.

The retroviral polypeptides and nucleic acids of the invention, and cells, vectors, transgenic animals, and compositions including such components can be packaged in packs, dispenser devices, and kits for administration to a subject, such as a mammal. For example, packs or dispenser devices that contain one or more unit dosage forms are provided. Typically, instructions for administration of the compounds will be provided with the packaging, along with a suitable indication on the label that the compound is suitable for treatment of an indicated condition. For example, the label may state that the active compound within the packaging is useful for treating a particular infectious disease, autoimmune disorder, tumor, or for preventing or treating other diseases or conditions that are mediated by, or potentially susceptible to, a subject's or mammalian immune response.

KITS

In an additional aspect, the present invention provides kits embodying the shear or stress resistant retroviruses of the invention, shear or stress resistant retrovirus envelope protein polypeptides or fragments thereof, and/or nucleic acids encoding shear or stress resistant retrovirus envelope protein polypeptides or fragments thereof, methods, and compositions of the invention described herein. Kits of the invention optionally comprise one or more of the following: (1) instructions for practicing the methods described herein, and/or for using the shear or stress resistant retroviruses, shear or stress resistant retroviruses envelope protein polypeptides or fragments thereof, and/or nucleic acids encoding shear or stress resistant retrovirus envelope protein polypeptides or fragments thereof or compositions, all of which are described herein; (2) one or more retrovirus of the invention, shear or stress resistant retrovirus envelope protein or nucleic acid of the invention or fragment thereof, individually, taken together, or as or in a composition or a component thereof, or retrovirus envelope protein nucleic acid of the invention or fragment thereof, individually or together, or as or in a composition or a component thereof; (3) a container for holding one or more of said shear or stress resistant retrovirus, retrovirus envelope protein, or retrovirus envelope protein nucleic acid, or respective fragment of any of the foregoing, individually or together, or compositions or components thereof, and (4) packaging materials.

In a further aspect, the present invention provides for the use of any composition or kit herein, for the practice of any method or assay herein, and/or for the use of any kit to practice any assay or method herein.

### EXAMPLES

### Example 1

### RECOMBINANT ECOTROPIC ENVELOPE GENES

Recombinant nucleic acid sequences corresponding to recombinant envelope regions (from conserved SfiI and ClaI sites in the 3′ of pol and in the ™ subunit of env, respectively) derived from six parental ecotropic murine leukemia retroviruses (292E, Friend virus strains 2, 7, 9, and 21, and Moloney murine leukemia virus (MLV) strain) were generated and cloned into a G1nBgSvNa, Moloney-based, backbone. G1nBgSvNa is a replication-defective Moloney-based vector encoding a nuclear form of β-galactosidase expressed from the Long Terminal Repeat (LTR) and a neomycin resistance gene expressed from a simian virus 40 promoter (SV40) in a G1 backbone (Lyons et al. (1995) Cancer Gene Ther. 2:273). Retroviral supernatants were generated by transient transfection of 293T cells with the recombinant vectors.

### Example 2

### DETERMIMNG SENSITIVITY OF RETROVIRUSES TO ULTRACENTRIFtUGATION

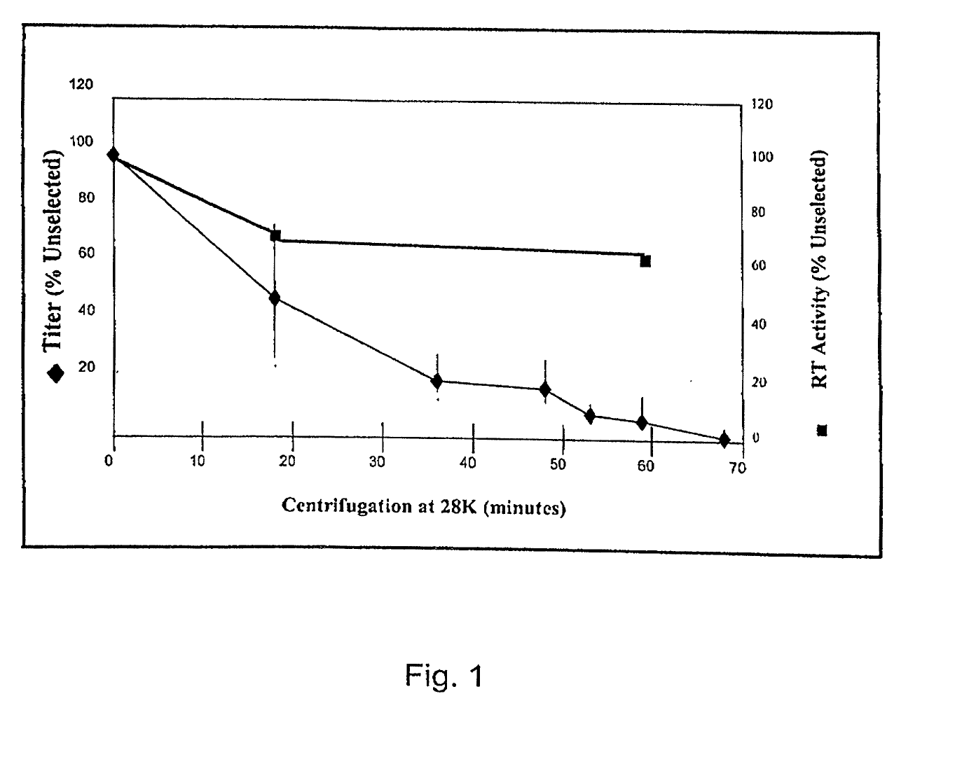

The sensitivity of MLV-based retroviral vectors to concentration by ultracentrifugation has been previously described; conditions for the selection of stress resistant viruses were based upon published conditions for concentration of VSV-G pseudotyped vectors (see, e.g., Emi et al. (1991) J. Virol. 6:1202; Burns et al. (1993) Proc. Nat'l Acad. Sci. USA 90:8033; Yee et al. (1994) Methods in Cell Biology 43:99) and were optimized using a replication defective vector encoding β-galactosidase, PE501/G1nBgSvNa (FIG. 1).

The retroviral supernatants were sealed in polyallomer tubes and centrifuged at about 120K×g (gravity) in a Beckman SW 28 rotor (Beckman Instruments, Fullerton, Calif.) for about 0-70 minutes. After centrifugation, the viral pellets were resuspended directly in the total volume of supernatant using a syringe. The samples were processed in this manner to ensure that the decreases in ability to transduce β-galactosidase were due to inactivation of virus rather than from lack of recovery in the pellets. The supernatant was left at 4° C. overnight to ensure complete solubilization. Control (noncentrifuged) virus was placed in centrifuge tubes and processed identically. β-galactosidase expression in cells transduced with the G1nBgSvNa vector was determined by incubation with the fluorescent substrate FDG (Molecular Probes, Eugene, Oreg.), used according to manufacturer's directions. Analysis was performed on a Coulter Elite flow cytometer.

Infectious titer of a retroviral solution comprising recombinant replication-competent retroviruses (RCRs) was typically measured by limiting-dilution vector rescue both before and after ultracentrifugation. Limiting-dilution vector rescue assay was performed in the following manner. Dilutions of supernatant in 0.25-0.5 log intervals were added to 3T3 or Mus dunni marker rescue cell lines stably expressing the β-galactosidase encoding vector (3Bg5A (3T3), DBg3A (M. dunni)) in the presence of 8 μg/ml polybrene and incubated for 4 hours. Forty-eight hours after transduction, the supernatant was collected from each dilution and used to infect naive cells (3T3 or M. dunni). The cells were fixed and stained for β-galactosidase expression 2-3 days after infection and scored microscopically for the presence of positive cells at each dilution.

Reverse transcriptase activity was determined on viral supernatants using a commercial kit (NEN Life Science Products, Boston, Mass.) used according to manufacturer's instructions. Reverse transcriptase activity was determined on fractions from each time point to show that recovery of active virus cores was only slightly reduced during the centrifugation process (right axis of FIG. 1).

Samples from each time point were then used to infect 3T3 cells, andf the percent of transduced cells positive for ′3-galactosidase was determined using a fluorescent substrate FDG detected by flow cytometry. The sensitivity of this vector to increasing times of centrifugation is shown in FIG. 1 (left axis). The percent of β-galactosidase positive cells decreased linearly with increasing times of centrifugation, with approximately 95 to 98% loss of activity by 68 minutes of centrifugation.

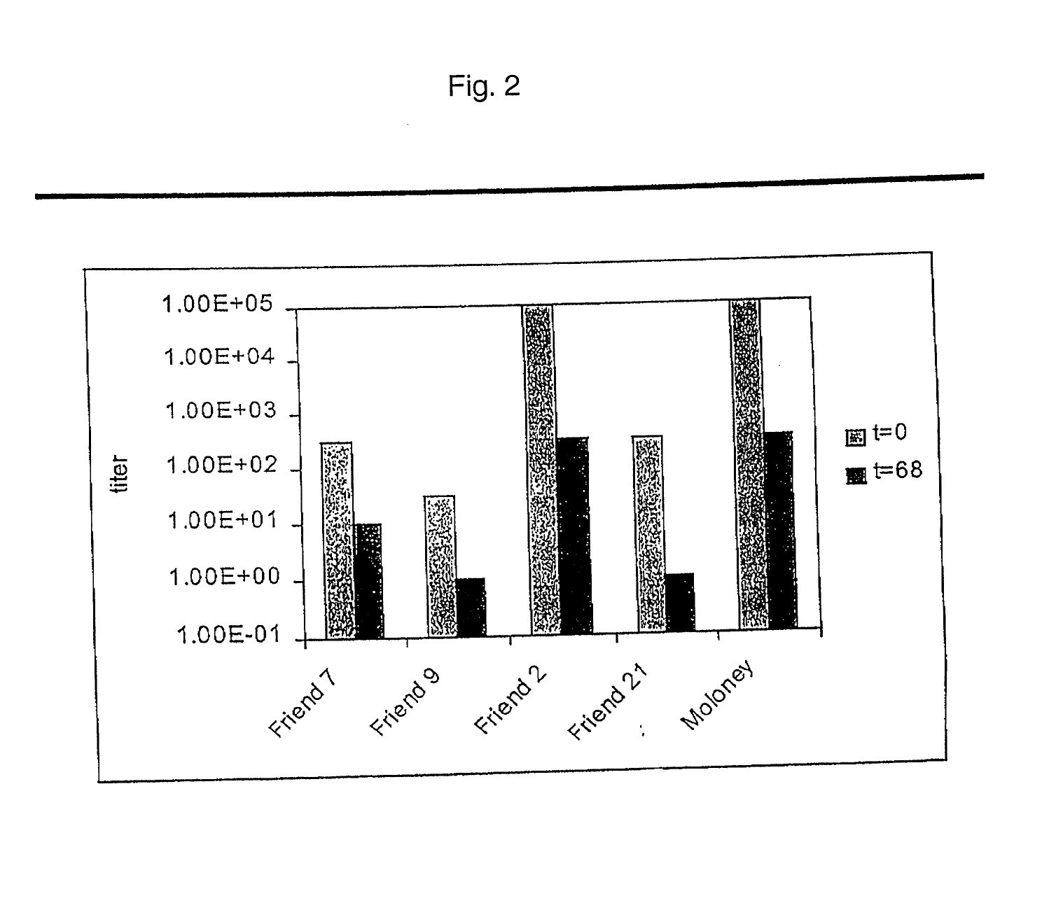

To determine if the parental control ecotropic retroviruses were similarly sensitive to ultracentrifugation, the envelope gene of each of the parental viruses was cloned into a Moloney RCR backbone, transfected into 293 cells to obtain stocks of the parental viruses, and titered before and after ultracentrifugation by limiting-dilution vector rescue. Titers obtained from the uncentrifuged parental RCR constructs ranged from 10^{1.5} to 10⁵ (FIG. 2); after ultracentrifugation, each of the parental RCR's exhibited a loss in infectious titer level of about 30- to about 100-fold relative to the infectious titer level of an identical (respective) parental RCR that was not subjected to ultracentrifugation. These results indicate that each of the parental viruses from which the stress and/or shear resistant clones were derived was sensitive to ultracentrifugation.

### Example 3

### SELECTION OF STRESS AND/OR SHEAR RESISTANT VIRUSES

After establishing the sensitivity of the parental representative retroviruses to ultracentrifugation, the set of recombinant retroviruses was subjected to three rounds of ultracentrifugation at about 120,000×g for about 68 or 80 minutes. Retroviral supernatants were sealed in polyallomer tubes and centrifuged at about 120,000×g in a Beckman SW 28 rotor for the selected time period. After centrifugation, the viral pellet was dispersed into the supernatant using a syringe, and left at 4° C. overnight to ensure complete solubilization. Control (non-centrifuged) retrovirus was placed in centrifuge tubes and processed identically.

After each round of centrifugation, retroviruses surviving each round of centrifugation in the retroviral supernatant were amplified and titered by limiting-dilution vector rescue on both 3T3 and Mus dunni cells. Each titer determination was done twice, first at log intervals to determine the appropriate range of dilution (data not shown), and then at half-log intervals to get a more accurate estimation of titer (see Table 3). Supernatants collected from both cell types were combined before each round of selection. After completion of three rounds of selection, pellets were collected from cells transduced with the stress- or shear resistant virus for isolation of recombinant envelope genes by PCR. Stress and/or shear resistance increased in both cell types examined after each initial round of screening, but after three cycles of ultracentrifugation/amplification as described above, the viral pool exhibited no loss in infectious titer after ultracentrifugation (see Table 3).

The results shown in Table 3 reflect the following. Supernatant of the RCR vectors comprising the recombinant (e.g., modified) env sequences was prepared by transient transfection of 293 cells with the env DNA. Samples were titered by limiting-dilution vector rescue on Mus dunni and 3T3 cells, with or without ultracentrifugation at about 120,000×g, as described above. Each titer determination was done twice as described above. After the third round of selection, the infectious titer of the retroviral pool (i.e., set of recombinant or modified retroviruses) was not reduced by such ultracentrifugation.

TABLE 3
Characterization of Stress- or Shear-Resistant Virus Population

| | Time | | | |
|---|---|---|---|---|
| | centrifuged | | Viral titer | |
| Cell line | (min) | 1^{st} round | 2^{nd} round | 3^{rd round} |
| Mus dunni | 0 | 10^{4.5} | 10^{4.5} | 10^{3.0} |
| Mus dunni | 68 | 10^{3.5} | 10^{4.0} | 10^{3.0} |
| 3T3 | 0 | 10^{4.5} | 10^{5.5} | 10^{3.0} |
| 3T3 | 68 | 10^{4.0} | 10^{5.0} | 10^{3.0} |

### Example 4

### SEQUENCE ANALYSIS OF STRESS- OR SHEAR RESISTANT VIRUSES

To characterize individual viral clones from this stress (and/or shear) resistant population, cell pellets were collected from Mus dunni cells transduced with the resistant viral supernatants. The envelope regions were cloned from the cellular genomic DNA using polymerase chain reaction (PCR). Sequence analysis of five recombinant or modified clones (clones 2B-17, 4-7, 4-4, 2B-13, and 2B-8) showed that each clone had a sequence distinct from the other four clones and from the parental sequences and known nucleic acid sequences encoding known MLV envelope proteins.

The recombinant or modified envelopes were cloned back into the RCR backbone and transfected into 293 cells to generate clonal viral stocks and analyzed for stress (and/or shear) resistance. The results of limiting-dilution vector rescue are shown in FIG. 3. Parental viruses run in the same experiment exhibited the same loss of about 30- to about 100-fold in titer that was observed previously (<1% of infectious titer retained at about 80 minutes (or more)). Three recombinant (modified) retroviral clones incorporating stress and/or shear resistant recombinant envelope protein polypeptides of the invention were resistant to the loss of infectious titer upon ultracentrifugation (clones 2B-17, 4-7, and 4-4) (see FIG. 3). (In FIG. 3, the infectious titer of each of the centrifuged samplesiAs' shown as a percentage of the infectious titer of each corresponding non-centrifuged “control” virus.) Recombinant retrovirus clone 2B-17 was most resistant to ultracentrifugation, showing no detectable loss in infectious titer level at 68 minutes and at up to 80 minutes or more of ultracentrifugation at about 120,000×g compared to the infectious titer level of the non-centrifuged recombinant clone 2B-17 (i.e., 2B-17 showed the same infectious titers level before and after ultracentrifugation). Recombinant retroviral clone 4-4 displayed an intermediate level of stress (and/or shear) resistance, showing no detectable loss of the level of infectious titer after 68 minutes of ultracentrifugation at about 120,000×g, and retaining a level of infectious titer after such ultracentrifugation of at least about 25% or 30% of the level of the infectious titer of a non-centrifuged recombinant clone 4-4 sample. Recombinant retroviral clone 4-7 was the least stress (or shear) resistant, but still retained approximately an infectious titer level of at least about 50% or 56% after 80 minutes (or more) of ultracentrifugation at about 120,000×g relative to the infectious titer level of non-centrifuged recombinant clone 4-7.

Two recombinant retroviral clones, 2B-13 and 2B-8, remained sensitive to ultracentrifugation at such forces (data not shown). The amino acid sequences of each of these stress or shear resistant retrovirus envelope protein polypeptides differ from one another and are unique with respect to naturally occurring or known retroviral or MLV envelope proteins. The nucleic sequences encoding these stress and/or shear resistant retroviral or MLV envelope proteins also differ from one another and are unique with respect to naturally occurring or known nucleotide sequence encoding retroviral or MLV envelope proteins. Each envelope protein sequence of a stress or shear resistant virus comprised a chimeric polypeptide sequence from at least two or more parental polypeptide sequences. Similarly, each polynucleotide sequence corresponding to each envelope protein sequence of a stress or shear resistant virus comprised a chimeric polynucleotide sequence from at least two or more parental polynucleotide sequences.

By comparison, retroviruses that did not incorporate a recombinant envelope polypeptide of the invention as an envelope protein typically showed a dramatic sensitivity to such ultracentrifugation conditions. See, e.g., results relating to MoMLV, Friend 7 and Friend 21 in FIG. 3. For example, the titer obtained by limiting-dilution vector rescue of a stock solution of a parental retrovirus incorporate a polypeptide of the invention was reduced substantially, e.g., by 30- to 100-fold, after being subjected to ultracentrifugation at about 120,000×g for about 68, 70, or 80 minutes, as compared to the titer of the same parental stock solution prior to such ultracentrifugation. Each parental virus was not able to maintain the level of infectious titer before and after ultracentrifugation. Shear (and/or stress) resistant viruses incorporating recombinant envelope protein polypeptides of the invention were up to 1000 times more stress and/or shear resistant than the representative parental retroviruses. The representative parental viruses (e.g., MoMLV, Fr. 7, Fr. 21) subjected to such ultracentrifugation conditions suffered from about 1 to 3 logs infectious titer decrease. See FIG. 2.

An additional experiment was done to determine whether stress or shear resistant viruses could be effectively concentrated by ultracentrifugation. Supernatants collected from two parental and three resistant clones were centrifuged at 120,000×g for about 68 minutes, similar to the conditions used during the selection procedures for stress or shear resistant retroviruses. Most of the culture supernatant was removed using a syringe, and the viral pellet was resuspended in a small volume of the residual supernatant, resulting in a suspension 30- to 65-fold more concentrated than the starting supernatants. The titers of the concentrated virus stock and of the uncentrifuged (starting) material determined using a limiting-dilution vector rescue are shown in Table 4. Neither parental virus gave detectably titer in the concentrated pellets, consistent with each parental virus' observed sensitivity to the procedure. In contrast, all three stress or shear resistant clones exhibited an increase in titer in the concentrated sample, ranging from one-half log to greater than two logs. This results shows that the stress or shear resistant viruses can be effectively concentrated and recovered. The degree of stress or shear resistance observed in our previous experiments did not correlate with the fold increase in titer obtained after ultracentrifugation. This set of experiments was designed to detect increases in titer as opposed to total recovery in the previous experiments. The results may be explained by differences in the ability to pellet and recover viruses. However, our ability to concentrate all three stress or shear resistant retrovirus clones by ultracentrifugation demonstrates these retroviruses have increased capacity for generating high-titer stocks by conventional physical methods.

TABLE 4
Stress or Shear Resistant Clones Can Be Concentrated by
Ultracentrifugation

| | Titer of | | |
|---|---|---|---|
| | untreated | Fold | |
| Virus | supernatant | concentrated | Titer after concentration |
| 2B-17 | 10^{5.5} | 31.6 | 10^{6.0} |
| 4-4 | 10^{5.0} | 49.0 | 10^{6.0} |
| 4-7 | 10^{5.0} | 63.2 | 10^{>7.5} |
| Moloney | 10^{3.0} | 65.3 | Below detection |
| Friend 9 | 10^{2.0} | 54.4 | Below detection |

Although all of the viable parental viruses were sensitive to ultracentrifugation, new recombinations or modifications of these parental viruses resulted in modified strains with improved stress or shear resistant properties. Furthermore, these strains have a greater ability to produce high-titer preparations than the parental viruses. These new retroviruses and retroviral vectors having properties that improve manufacturability and manufacturing yield compared to known retroviruses and retroviral vectors are particularly useful in retroviral applications where highly purified and high-titer preparations are desired, such as gene therapy.

While the foregoing invention has been described in some detail for purposes of clarity and understanding, it will be clear to one skilled in the art from a reading of this disclosure that various changes in form and detail can be made without departing from the true scope of the invention. For example, all the techniques, methods, compositions, apparatus and systems described above may be used in various combinations. All publications, patents, patent applications, or other documents cited in this application are incorporated by reference in their entirety for all purposes to the same extent as if each individual publication, patent, patent application, or other document were individually indicated to be incorporated by reference in its entirety for all purposes.

What is claimed is:

1. An isolated or recombinant nucleic acid, comprising a polynucleotide sequence selected from the group consisting of:
(a) SEQ ID NOS:1-3 and SEQ ID NOS:9-10, or a complementary polynucleotide sequence thereof;
(b) a polynucleotide sequence encoding a polypeptide selected from SEQ ID NOS:4-8, or a complementary polynucleotide sequence thereof;
(c) a polynucleotide sequence that hybridizes under highly stringent conditions over substantially the entire length of polynucleotide sequence (a) or (b); and
(d) a polynucleotide sequence comprising a fragment of (a), (b), or (c), that fragment encodes all or a part of a stress or shear resistant retrovirus envelope protein.

2. The nucleic acid of claim 1, which polypeptide comprises all or part of a stress or shear resistant retrovirus envelope protein.

3. The nucleic acid of claim 1, wherein the stress or shear resistant retrovirus envelope protein is capable of withstanding ultracentrifugation at a force of at least about 120,000×g for at least about 60 minutes.

4. The nucleic acid of claim 3, wherein a modified retrovirus comprising said stress or shear resistant retrovirus envelope protein withstands ultracentrifugation at a force of at least about 120,000×g for at least about 60 minutes, wherein said modified retrovirus has an infectious titer level following said ultracentrifugation that is at least about 30%, 40%, or 50% of an infectious titer level of an identical modified retrovirus comprising said stress or shear resistant retrovirus envelope protein that has not undergone said ultracentrifugation.

5. The nucleic acid of claim 2, wherein the encoded polypeptide comprises an amino acid sequence selected from the group consisting of: SEQ ID NO:4 to SEQ ID NO:6.

6. The nucleic acid of claim 2, comprising a polynucleotide sequence selected from the group consisting of: SEQ ID NO:1 to SEQ ID NO:3.

7. An isolated or recombinant nucleic acid comprising a polynucleotide sequence encoding a polypeptide, the polypeptide comprising an amino acid sequence comprising at least 500 contiguous amino acids of any one of SEQ ID NOS:4-8.

8. The nucleic acid of claim 7, wherein the encoded polypeptide comprises at least 600 contiguous amino acid residues of any one of SEQ ID NOS:4-8.

9. The nucleic acid of claim 8 wherein the encoded polypeptide is 671, 672, or 675 amino acids in length.

10. The nucleic acid of claim 7, wherein the encoded polypeptide comprises a stress or shear resistant retrovirus envelope protein.

11. A cell comprising the nucleic acid of claim 1 or 7.

12. The cell of claim 11, wherein the cell expresses a polypeptide encoded by the nucleic acid.

13. A vector comprising the nucleic acid of claim 1, 7, 45, or 49.

14. The vector of claim 13, wherein the vector comprises a plasmid, a cosmid, a phage, or a virus.

15. The vector of claim 13, wherein the vector is a retrovirus.

16. The vector of claim 13, wherein the vector is an expression vector.

17. A cell transduced by the vector of claim 13.

18. A composition comprising the nucleic acid of claim 1 or 7 and an excipient.

19. The composition of claim 18, wherein the excipient is a pharmaceutically acceptable excipient.

20. An isolated or recombinant polypeptide encoded by the nucleic acid of acid claim 1 or 7.

21. The polypeptide of claim 20, comprising an amino acid sequence selected from the group consisting of: SEQ ID NOS:4-8.

22. The polypeptide of claim 21, which polypeptide comprises a stress or shear resistant retrovirus envelope protein.

23. The polypeptide of claim 22, wherein a modified retroviruscomprising said stress or shear resistant retrovirus envelope protein is capable of withstanding ultracentrifugation at a force of at least about 120,000×g for at least about 60 minutes.

24. The polypeptide of claim 23, wherein a supernatant comprising said retrovirus has an infectious titer level following said ultracentrifugation that is at least about 30%, 40%, or 50% of an infectious titer level of a supernatant comprising an identical retrovirus comprising said stress or shear resistant retrovirus envelope protein that has not undergone said ultracentrifugation.

25. A polypeptide comprising at least 500 contiguous amino acids of a protein encoded by a coding polynucleotide sequence, the polynucleotide sequence selected from the group consisting of:
(a) SEQ ID NOS:1-3 and SEQ ID NOS:9-10;
(b) a coding polynucleotide sequence that encodes a first polypeptide selected from SEQ ID NOS:4-8; and
(c) a complementary polynucleotide sequence that hybridizes under highly stringent conditions over substantially an entire length of a polynucleotide sequence of (a) or (b).

26. The polypeptide of claim 25, comprising at least 600 contiguous amino acids of the encoded protein.

27. The polypeptide of claim 26, wherein the polypeptide is at least 671, 672, or 675 amino acids in length.

28. The polypeptide of claim 25, which polypeptide comprises a stress or shear resistant retrovirus envelope protein.

29. The polypeptide of claim 28, wherein the stress or shear resistant retrovirus envelope protein is capable of withstanding ultracentrifugation at a force in excess of at least about 120,000×g for at least about 60 minutes such that a retrovirus comprising said stress or shear resistant retrovirus envelope protein exhibits an infectious titer level that is at least about 30%, 40%, or 50% of an infectious titer level of an identical retrovirus comprising said stress or shear resistant retrovirus envelope protein that has not undergone said ultracentrifugation.

30. The polypeptide of claim 29, comprising at least 600 contiguous amino acid residues of any one of SEQ ID NOS:4-6.

31. An isolated or recombinant polypeptide comprising an amino acid sequence comprising at least 650 contiguous amino acids of any one of SEQ ID NOS:4-8.

32. The polypeptide of claim 31, which polypeptide comprises a stress or shear resistant retrovirus envelope protein polypeptide or a fragment thereof.

33. The polypeptide of claim 32, wherein the stress or shear resistant retrovirus envelope protein polypeptide is capable of withstanding ultracentrifugation in excess of at least about 120,000×g for at least about 60 minutes.

34. The polypeptide of claim 33, wherein a solution comprising a modified retrovirus comprising said stress or shear resistant retrovirus envelope protein polypeptide has an infectious titer level that is at least about 30%, 40%, or 50% of the infectious titer level of a solution comprising an identical modified retrovirus comprising said stress or shear resistant retrovirus envelope protein polypeptide that has not undergone said ultracentrifugation.

35. The polypeptide of claim 20, 25, or 31, further comprising a secretion/localization sequence.

36. The polypeptide of claim 20, 25, or 31, further comprising a polypeptide purification subsequence.

37. The polypeptide of claim 36, wherein the purification subsequence is selected from the group consisting of: an epitope tag, a FLAG tag, a polyhistidine tag, and a GST fusion.

38. The polypeptide of claim 20, 25, or 31, further comprising a Met at the N-terminus.

39. The polypeptide of claim 20, 25, or 31, comprising a modified amino acid.

40. The polypeptide of claim 39, wherein the modified amino acid is selected from the group consisting of: a glycosylated amino acid, a PEGylated amino acid, a farnesylated amino acid, an acetylated amino acid, and a biotinylated amino acid.

41. A composition comprising the polypeptide of claim 20, 25, or 31 and an excipient.

42. The composition of claim 41, wherein the excipient is a pharmaceutically acceptable excipient.

43. A stress or shear resistant retrovirus comprising an envelope protein selected from SEQ ID NO:4 to SEQ ID NO:6.

44. The stress or shear resistant retrovirus of claim 43, further comprising a therapeutic or prophylactic gene construct.

45. A nucleic acid that comprises a unique subsequence in a nucleic acid selected from any of SEQ ID NOS:1-3 and SEQ ID NOS:9-10, wherein the unique subsequence is unique as compared to a nucleic acid corresponding to a naturally occurring or known MLV nucleic acid sequence or MLV nucleic acid sequence present in GenBank.

46. A polypeptide that comprises a unique amino acid subsequence in a polypeptide selected from any of SEQ ID NOS:4-8, wherein the unique amino acid subsequence is unique as compared to an amino acid subsequence polypeptide corresponding to a naturally occurring or known MLV envelope protein or MLV envelope protein present in GenBank.

47. A target nucleic acid that hybridizes under stringent conditions to a unique coding oligonucleotide that encodes a unique amino acid subsequence in a polypeptide selected from any of SEQ ID NOS:4-8, wherein the unique amino acid subsequence is unique as compared to an amino acid subsequence of a naturally occurring or known MLV envelope protein or MLV envelope protein present in GenBank.

48. The nucleic acid of claim 47, wherein the stringent conditions are selected such that a perfectly complementary oligonucleotide to the unique coding oligonucleotide hybridizes to the unique coding oligonucleotide with at least a 5× higher signal to noise ratio than for hybridization of the perfectly complementary oligonucleotide to a control nucleic acid corresponding to a naturally occurring or known MLV envelope protein or MLV envelope protein present in GenBank.

49. A modified or recombinant nucleic acid that encodes a stress or shear resistant retrovirus envelope polypeptide capable of withstanding ultracentrifugation at a force of at least about 120,000×g for at least about 60 minutes.

50. A stress or shear resistant retrovirus envelope polypeptide comprising an amino acid sequence that has at least about 90% amino acid sequence identity with an amino acid sequence of at least one of SEQ ID NOS:4-6.

51. The stress or shear resistant retrovirus envelope polypeptide, wherein said polypeptide is not the polypeptide represented by SEQ ID NO:7 or SEQ ID NO:8.

52. The stress or shear resistant retrovirus envelope polypeptide of claim 50, said polypeptide comprising an amino acid sequence that has at least about 95% amino acid sequence identity with an amino acid sequence of at least one of SEQ ID NOS:4-6.

53. The stress or shear resistant retrovirus envelope polypeptide of claim 50, wherein said polypeptide is capable of withstanding ultracentrifugation at a force of at least about 120,000×g for at least about 30 minutes.

54. The stress or shear resistant retrovirus envelope polypeptide of claim 50, wherein the amino acid sequence is not encoded by a polynucleotide sequence represented by any of GenBank Nucleic Acid Accession Nos. Z35109, K02714, Y13893, D88386, Z11128, M93134, X02794, AF288940, U94692, AF030173, S80869, S77017, S77015, S77012, J01998, AF169256, and AH000833.

55. A polypeptide which comprises an amino acid sequence encoded by a coding polynucleotide sequence, the coding polynucleotide sequence selected from the group of:
(a) a polynucleotide sequence selected from at least one of SEQ ID NOS:1-3 and SEQ ID NOS:9-10, or a complementary nucleic acid sequence thereof;
(b) a polynucleotide sequence that encodes a polypeptide selected from any of SEQ ID NOS:4-8;
(c) a polynucleotide sequence which hybridizes under stringent conditions over substantially the entire length of a polynucleotide sequence (a) or (b);
(d) a polynucleotide sequence encoding a polypeptide comprising an amino acid sequence that is substantially identical over at least about 550 contiguous amino acid residues of any one of SEQ ID NOS:4-8, provided said amino acid sequence is not a protein sequence encoded by a nucleotide sequence represented by any of GenBank Accession Nos. Z35109, K02714, Y13893, D88386, Z11128, M93134, X02794, AF288940, U94692, AF030173, S80869, S77017, S77015, S77012, J01998, AF169256, and AH000833; and
(e) a polynucleotide sequence encoding a stress or shear resistant retrovirus envelope polypeptide, which polynucleotide sequence has at least about 95% identity to at least one polynucleotide sequence of (a), (b), (c), or (d).

56. The polypeptide of claim 55, the polypeptide comprising an amino acid sequence of any one of SEQ ID NOS:4-8.

57. The polypeptide of claim 55, wherein said polypeptide is capable of withstanding centrifugation at a force of at least about 120,000×g for at least about 60 minutes.

58. The polypeptide of claim 57, said polypeptide comprising the amino acid sequence of any one of SEQ ID NOS:4-6.

59. An isolated or recombinant variant polypeptide of a parent polypeptide, the variant polypeptide comprising at least one substitution from the group of Q454K and S469N, wherein the parent polypeptide comprises amino acid residue 4 to amino acid residue 615 of SEQ ID NO:11.

60. The isolated or recombinant variant polypeptide of claim 59, further comprising at least one additional substitution selected from the group of R56Q, K147R, V209A, Q210K, A228T, Q289L, A378T, and T413A.

61. The isolated or recombinant variant polypeptide of claim 59, wherein the parent polypeptide comprises amino acid residue 4 to amino acid residue 671 of SEQ ID NO:11.

62. The nucleic acid molecule of claim 1, 7, or 45, wherein said nucleic acid is a DNA molecule or an mRNA molecule.

63. A nucleic acid that encodes a stress or shear resistant retrovirus envelope polypeptide, said polypeptide has at least about 95% amino acid sequence identity with an amino acid sequence of at least one of SEQ ID NOS:4-6.

64. A stress or shear resistant retrovirus comprising all or part of the stress or shear resistant retrovirus envelope polypeptide of claim 28, 50, or 55.

65. A composition comprising at least one polypeptide of claim 28, 50, or 55 and an excipient.

66. A replicative retrovirus comprising the nucleic acid of claim 1, 7, 49, or 63.

67. A composition comprising two or more nucleic acids of claim 1, 7, 49, or 63.

68. A method of producing a polypeptide, the method comprising:
introducing into a population of cells a nucleic acid of claim 1, 7, 49, or 63, the nucleic acid operatively linked to a regulatory sequence effective to produce the encoded polypeptide; and
culturing the cells in a culture medium to produce the polypeptide.

69. A method of producing a polypeptide, the method comprising:
introducing into a population of cells an expression vector comprising the nucleic acid of claim 1, 7, 49, or 63; and culturing the cells in a culture medium to produce the polypeptide encoded by the expression vector.

70. A method of producing a polypeptide, the method comprising:
infecting a population of cells with a retrovirus comprising the nucleic acid of claim 1, 7, 49, or 63; and
culturing the cells in a culture medium to produce the polypeptide encoded by the nucleic acid.

71. A method of producing a stress or shear resistant retrovirus, the method comprising:
infecting a population of cells with a retrovirus comprising the nucleic acid of claim 1, 7, 49, or 63; and
culturing the cells in a culture medium to produce a plurality of amplified retroviruses comprising the polypeptide encoded by the nucleic acid.
